(19) United States
(12) Patent Application Publication
(10) Pub. No.: US 2010/0249064 A1
(43) Pub. Date: Sep. 30, 2010

(54) METHODS AND COMPOSITIONS FOR TREATING DISEASES AND CONDITIONS INVOLVING HIGHER MOLECULAR WEIGHT HYALURONAN

(75) Inventors: Patrick A. Singleton, Chicago, IL (US); Joe G.N. Garcia, Chicago, IL (US)

Correspondence Address: FULBRIGHT & JAWORSKI L.L.P., 600 CONGRESS AVE., SUITE 2400, AUSTIN, TX, 78701, US

(73) Assignee: University of Chicago, Chicago, IL (US)

(21) Appl. No.: 12/676,793

(22) PCT Filed: Sep. 5, 2008
(86) PCT No.: PCT/US2008/075437

§ 371 Date: Mar. 5, 2010

Related U.S. Application Data
(60) Provisional application No. 60/970,857, filed on Sep. 7, 2007.

(51) Int. Cl. A61K 31/7008; A61P 9/10; A61P 9/00

(52) U.S. Cl. 514/62

(57) ABSTRACT

The present invention concerns methods and compositions involving hyaluronan that has been substantially purified to enrich for hyaluronan with a molecular weight above 500 kilodaltons. This hyaluronan can be used for diseases and conditions characterized or caused by increased vascular permeability or angiogenesis. The higher molecular weight hyaluronan restores vascular integrity and inhibits angiogenesis in embodiments of the invention.

This application claims priority to U.S. Provisional Patent Application Ser. No. 60/970,857 filed on Sep. 7, 2007, which is hereby incorporated by reference.

This invention was made with government support under F32 HL68472 and PPG HL 58064 awarded by the National Institutes of Health. The government has certain rights in the invention.

### BACKGROUND OF THE INVENTION

I. Field of the Invention

The present invention relates generally to the fields of pulmonary disease and conditions, particularly those involving vascular permeability, and angiogenesis-related diseases and conditions. In a more particular embodiment, it concerns methods and compositions involving purified high molecular weight hyaluronan (also called hyaluronic acid or hyaluronate) for the prevention and treatment of these diseases and conditions.

II. Background

There are a number of diseases and conditions that involve increased vascular permeability or angiogenesis. For instance, increased vascular permeability is observed in Acute Lung Injury/Acute Respiratory Distress Syndrome (ALI/ARDS), both of which are devastating consequences of systemic inflammatory conditions such as sepsis. They afflict almost 200,000 people a year in the US with 75,000 deaths. Acute lung injury (ALI) is a major cause of morbidity and mortality in critically ill patients. A defining feature of ALI is a disruption of the endothelial cell (EC) barrier lining the pulmonary vasculature that results in leakage of fluid, protein and cells into the airspaces of the lungs. One important extracellular matrix component, hyaluronan (HA), and its cell surface receptor, CD44, has been implicated in normal EC function and angiogenesis.

A role for HA has been investigated in the context of lung inflammatory processes or elastic fiber injury. For instance, U.S. Pat. No. 5,633,003 discusses treatments for emphysema. This document does not disclose using a composition with purified hyaluronan in which high molecular weight hyaluronan has been enriched. The destruction of elastin is central to emphysema's disease pathogenesis (Shifren et al., 2006). Moreover, emphysema is a disease involving bronchoconstriction and does not implicate vascular barrier regulation, which is in contrast to diseases such as ALI and ARDS (Shifren et al., 2006). Likewise, bronchoconstriction is not a clinically significant feature of ALI/ARDS (Hasleton et al., 1999).

PCT Application 00/193846 talks about hyaluronan in the context of elastin diseases, as does U.S. Patent Publication 2002/0086852. However, elastin diseases are physiologically different from diseases and conditions involving vascular permeability. Previous studies have conclusively demonstrated that elastin degradation, and not vascular permeability, is a key step in elastin diseases such as chronic obstructive pulmonary disease (Shifren et al. 2006). Neutrophils have been shown to secrete potent elastases and studies have shown that neutrophils can be beneficial in the context of ALI/ARDS, and not detrimental. (Sivan et al., 1990).

Moreover, applications of hyaluronan for the treatment of bronchoconstriction do not provide evidence for use in the treatment of vascular permeability. Endothelium can release endothelins, which causes bronchoconstriction, but in ARDS/ALI, the endothelium releases nitric oxide, which is vasorelaxive. Many of these references do not distinguish between different sizes of hyaluronan molecules and teach the use of hyaluronan that is less than 500 kilodaltons in weight.

While the role of HA/CD44 in lung inflammatory processes has been extensively studied, little is known about HA/CD44 regulation of pulmonary vascular permeability. As ALI, ARDS and other disorders involve increased vascular permeability, further information about the physiology might yield new and effective treatment options for conditions associated with increased vascular permeability. Agents that can enhance or restore vascular integrity will have important clinical utility in the treatment of such diseases or conditions.

Angiogenesis is an essential phenotype in a number of physiologic and pathologic processes including tumor progression (Arnold et al., 1991; Folkman 1995; Risau, 1997). In a series of now classical experiments, Folkman and colleagues demonstrated that solid tumors cannot grow larger than 2-3 mm in diameter unless they induce their own blood supply (Arnold et al., 1991; Folkman et al., 1991). The expression of the angiogenic phenotype is a complex process that depends on a number of cellular and molecular events in space and time (Arnold et al., 1991; Folkman 1995; Risau, 1997). Some of these events include degradation of the surrounding basement membrane, migration of endothelial cells through the connective tissue stroma, cell proliferation, the formation of tube-like structures, and the maturation of these endothelial-lined tubes into new blood vessels (Arnold et al., 1991; Folkman 1995; Risau, 1997). Recent therapeutic interventions for the inhibition of cancer progression include drugs that target tumor angiogenesis (Cardones et al., 2006; Dhanabal et al., 2005; Gaya et al., 2005; Glade-Bender et al., 2003). Neutralizing antibodies to vascular endothelial growth factor (VEGF) such as Bevacizumab™ have shown promise in the treatment of certain cancers (Cardones et al., 2006; Dhanabal et al., 2005; Gaya et al., 2005; Glade-Bender et al., 2003). However, the cost of Bevacizumab™ is high and there are significant side effects. Therefore, the discovery of a potent, cost effective, anti-angiogenic agent with minimal side effects would be of immense importance in cancer therapy.

There remains a need in the art for additional compositions and methods for preventing and/or treating conditions and diseases involving vascular permeability, as well as those conditions and diseases involving angiogenesis.

### SUMMARY OF THE INVENTION

The present invention is based on a diverse but related data set that demonstrates a number of novel insights into the physiological role of high molecular weight hyaluronan in the settings of vascular permeability and angiogenesis. The data indicate that high molecular weight hyaluronan increased transendothelial monolayer electrical resistance (TER), while low molecular weight hyaluronan induced biphasic TER changes ultimately resulting in endothelial cell barrier disruption. In addition, the data show that CD44, whose ligand is high molecular weight hyaluronan, is an important regulator of vascular integrity. There is also information showing that high molecular weight hyaluronan and low molecular weight hyaluronan differentially regulate HABP2 expression and activity. Moreover, the data indicate that high molecular weight hyaluronan can have an anti-angiogenic effect. Therefore, the present invention concerns methods and compositions involving hyaluronan above a certain weight and not low molecular weight hyaluronan nor a mixture of high and low molecular weight hyaluronan.

In some embodiments of the invention, there are methods for regulating vascular permeability in a subject having symptoms of or diagnosed with a disease or condition involving increased vascular permeability.

In other embodiments there are methods for treating a vascular permeability-related disease in a subject.

Embodiments of the invention may be implemented on subjects who have a disease or condition involving increased vascular permeability or who are at risk for such diseases or conditions. Vascular permeability refers to the capacity of the wall of a blood vessel to allow small molecules or cells to pass through. Endothelial cells make up blood vessel walls. Diseases or conditions that are characterized by or caused by an increase in vascular permeability include, but are not limited to, acute respiratory distress syndrome (ARDS), acute lung injury (ALI), sepsis, radiation pneumonitis, tumors, macular degeneration, capillary leakage syndrome, or atherosclerosis. Such diseases and conditions may be referred to as “vascular permeability-related diseases and conditions.” In particular embodiments, methods involve protecting a subject from radiation pneumonitis.

Additional methods concern treating a lung disease or condition involving barrier disruption of the endothelial cells in the lungs. A “lung disease or condition” refers to a physiological disease or condition that afflicts the lung, regardless of whether the disease or condition is caused by an affliction specifically in the lungs. However, in some embodiments of the invention, methods may be applied in the context of lung diseases or conditions caused by an affliction in the lungs.

Additional embodiments of the invention involve methods for inhibiting angiogenesis in a subject who exhibits symptoms of or has been diagnosed with an angiogenesis-related disease or condition. An angiogenesis-related disease or condition includes, but is not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, Rubeosis; Osler-Webber Syndrome; myocardial angiogenesis; plaque neovascularization; telangiectasia; hemophiliac joints; angiofibroma; and wound granulation. In certain embodiments, an angiogenesis-related disease or condition may also be a vascular permeability-related disease or condition, and vice versa.

In some embodiments the method comprises administering to the subject an effective amount of a composition comprising substantially purified hyaluronan, wherein the hyaluronan is at least about 95% pure hyaluronan with a molecular weight greater than about 500 kilodaltons. In other embodiments, methods may involve administering to a patient in need of such treatment an effective amount of a composition comprising purified hyaluronan, wherein the hyaluronan has a molecular weight above about 500 kilodaltons. In further embodiments, methods may involve administering to the subject a composition comprising substantially purified hyaluronan, wherein the hyaluronan is at least about 95% pure hyaluronan with a molecular weight greater than about 500 kilodaltons. It is specifically contemplated that in some embodiments a composition comprises substantially purified hyaluronan, wherein the hyaluronan is at least about 95% pure hyaluronan with a molecular weight greater than about 1000 kilodaltons.

In some embodiments of the invention, methods include identifying a subject or patient in need of a treatment. Identifying a subject or patient in need of the treatment may be accomplished by diagnosing the subject with a vascular permeability-related disease or condition, identifying a subject as being at risk for a vascular permeability-related disease or condition, determining that the subject has one or more symptoms of a vascular-permeability disease or condition, confirming a diagnosis that the subject has a vascular permeability-related disease or condition or that the subject has one or more symptoms of a vascular-permeability disease or condition. In other embodiments of the invention, instead of a vascular-permeability disease or condition, the subject is determined to be in need of a treatment for an angiogenesis-related disease or condition.

Methods and compositions of the invention may be implemented in the context of any subject, including mammalian subjects such as humans.

Methods and compositions of the invention involve hyaluronan, particularly hyaluronan that is above a particular molecular weight because the inventors data indicated that hyaluronan in the lower molecular weight range caused a different effect than hyaluronan in a higher weight range. Consequently, embodiments of the invention involve hyaluronan that is about or at least about 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 kilodaltons (kD) or more, or any range derivable therein. In certain embodiments, the hyaluronan has a molecular weight of at least about one million daltons, which is conventionally considered “high molecular weight (HMW)” hyaluronan. Any embodiment of the invention involving hyaluronan may be implemented specifically with high molecular weight hyaluronan. In other embodiments, a composition has purified away from or does not contain a detectable amount of hyaluronan with a molecular weight below about 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 daltons. In specific embodiments, methods and compositions involve a hyaluronan composition that contains less than about 1, 2, 3, 4, or 5% hyaluronan (by weight) having a molecular weight below 500 kDaltons, or any range derivable therein. In further embodiments, methods and compositions involve a hyaluronan composition that contains less than about 1, 2, 3, 4, or 5% hyaluronan (by weight) having a molecular weight below 1000 kDaltons, or any range derivable therein. A “detectable amount” of a hyaluronan refers to an amount that can be detected according to a 4-20% SDS-PAGE gel stained with Alcian blue and silver staining, according to Singeleton et al., 2006, which is hereby incorporated by reference, or an ELISA-like competitive binding assay with a known amount of fixed HA and biotintylated HA binding peptide (HABP) as the indicator.

In specific embodiments, methods involve a composition in which the hyaluronan is about or at least about 90, 95, 96, 97, 98, 99, 99.5% pure or homogeneous (or any range derivable therein) with respect to the hyaluronan content by weight, as compared to other cells and cellular components that it was purified away from. The term “substantially purified” refers to a composition of which at least 95% of the hyaluronan by weight has the indicated characteristics.

It is contemplated that components may be added to any hyaluronan composition and that purity is referenced only with respect to cells and cellular components that the hyaluronan is being purified away from, such as nucleic acids, chondroitin sulfate, lower molecular weight hyaluronan, proteins, and/or other cellular debris (referred to as “biological macromolecules”) and contaminants. Purity can be measured by any appropriate standard method known in the art, for example, by column chromatography, polyacrylamide gel electrophoresis, ELISA, or HPLC analysis.

In other embodiments of the invention, a composition with hyaluronan does not contain a detectable amount of nucleic acids, chondroitin sulfate, hyaluronan below a particular molecular weight, and/or any endotoxins, as determined when evaluating 10 ng to 100 ng hyaluronan with an SDS-PAGE gel stained with the appropriate stain or in an ELISA assay. Alternatively, a composition may contain less than about or at most about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 950, 960, 970, 980, 990, 1000 pg or ng of contaminating nucleic acid and/or protein per 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 950, 960, 970, 980, 990, 1000 μg or mg of HA, or any range derivable therein. For endotoxin, a composition may contain at most about 0.01, 0.02, 0.03, 0.04, 0.05, 0.06, 0.07 EU/mg.

In further embodiments of the invention, a composition contains hyaluronan that was purified using a size exclusion filter or gel filtration chromatography. The hyaluronan may also have been subject to a proteinase (such as proteinase K), boiling, or heat above room temperature.

Compositions of the invention may be administered to patients via any route used to introduce therapy to patients. Such routes include, but are not limited to, administration intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, via nebilizer, or via a lavage, or various combinations thereof. In specific embodiments, the composition is administered to the subject by inhalation. In particular embodiments, the composition is administered to the subject as an aerosol. Other examples of routes of administration involve a nebilizer. Additionally, the composition may be administered directly to the area affected by the increased vascular permeability or angiogenesis.

In certain embodiments, a composition is provided to endothelial cells in the subject. In further embodiments, the composition is administered to the tumor by intratumoral injection, by administration to the tumor bed, by administration to an area proximal to the tumor.

The compositions may be formulated in a pharmaceutically acceptable composition. In certain embodiments, a preservative and/or stabilizer is included in the composition.

Furthermore, in embodiments of the invention, methods may involve compositions containing about, at least about, or at most about 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 ng, μg or mg of HA (or any range derivable therein), which may be in about, at least about, or at most about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 10, 11, 12, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μl or ml (or any range derivable therein). Moreover, such amounts may be administered to a subject as that much hyaluronan/kg body weight of the subject. For example, a subject may be administered an amount in the range of about 1 μg/kg and about 1 mg/kg. In certain embodiments, the amount given to a subject is about, at least about, or at most about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/kg or mg/kg, or any range derivable therein. These amounts may be prescribed on a per administration basis or on a daily basis (for example on a μg/kg body weight/day basis).

Such amounts can be administered daily, though other dosing regimens are contemplated. It is contemplated that compositions of the invention may be administered just a single time or multiple times. In certain embodiments of the invention, a composition is administered 1, 2, 3, 4, 5, 6 or more times, or any range derivable therein. It is contemplated that a preventative or treatment regimen may involve multiple administrations over 1, 2, 3, 4, 5, 6, and/or 7 days or 1, 2, 3, 4, or 5 weeks, and/or 1, 2, 3, 4, 5, 6, 7, 8, 9, 10, 11, and/or 12 months, or any range derivable therein. Moreover, any such regimen may be repeated after a certain amount of time has passed or when symptoms of the disease or condition become noticeable or more severe.

In some embodiments a patient is also given one or more other treatments used for treating the disease or condition. Examples of such treatments include administration of corticosteroids (such as methylprednisolone) or applying airway pressure release ventilation, or applying other ventilation techniques such as low tidal volume ventilation. Other examples of relevant treatment, such as a treatment for cancer, include cancer chemotherapeutics, radiation, and/or immunotherapy. A patient may have been treated previously or may be treated concurrently or in the future with such treatments.

The present invention also concerns methods of screening for HABP2 modulators. In certain embodiments, methods involve a) contacting a cell having a nucleic acid encoding HABP2 with a candidate compound; and, b) measuring the level of HABP2 expression or activity in the cell, wherein the candidate compound is a candidate modulator of HAPB2 if the level of HABP2 expression or activity changes compared to a cell having a nucleic acid encoding HABP2 that is not contacted with a candidate compound. In other embodiments, there are methods involving HABP2 protein, which may not be in a cell, and contacting the protein with a candidate compound. Additional steps may involve determining if the candidate compound alters HABP2 activity or binds to HABP2.

In some embodiments, the level of HABP2 expression in the cell is measured. In other embodiments, HABP2 expression is measured by measuring the amount of HABP2 protein in the cell. In particular embodiments, HABP2 activity is measured. HABP2 has serine protease activity and it has the ability to form a complex with C1 NH. In certain embodiments, HABP2 activity is measured by fibrinolysis assay, coagulation assay, protease assay, surface plasmon resonance binding assay, fluorescence polarization, radioactive tracer assay, and/or homogeous time-resolved fluorescence assay. In specific embodiments, HABP2 complex formation with C1NH is used to evaluate HABP2 activity, such as by detecting a loss of binding to or complex formation with C1NH.

Methods may further involve evaluating a candidate modulator that decreases HABP2 expression or activity as a treatment for vascular permeability or to inhibit angiogenesis. For instance, candidate modulators may be evaluated in cells, tissues, or animal to evaluate any relevant properties. Animal models may be employed to evaluate the candidate modulators.

It is contemplated that the candidate substance may be a small molecule, nucleic acid, or polypeptide in some embodiments of the invention. It is also contemplated that methods may be implemented in high throughput assays or with arrays.

Additional methods of the invention include treatment or prevention methods involving HABP2 inhibitors. Methods include treating or preventing certain diseases or conditions. In some embodiments, methods involve identifying a patient in need of prevention or treatment.

inflammatory diseases or conditions of the lungs. It is contemplated that in some embodiments the inflammatory condition or disease afflicts the lungs, such as ALI, VILI, or ARDS. Other embodiments concern methods for preventing or treating vascular permeability diseases or conditions. In further embodiments, there are methods for inhibiting angiogenesis.

Embodiments of the invention concern methods for treating or preventing an inflammatory disease or condition of the lungs comprising administering an effective amount of an HABP2 inhibitor to a patient. In some embodiments of the invention, methods involve patients who are at risk for ALI or ARDS or with symptoms of ALI or ARDS. In certain cases, a patient has been diagnosed with ALI or ARDS. At risk patients include, but are not limited to, patients with sepsis or symptoms of sepsis, patients with pneumonia or symptoms of pneumonia, patients with severe bleeding because of an injury to the body, patients who have a severe injury to the chest or head, patients who have breathed harmful fumes or smoke, and patients inhaled vomit, patients who have had multiple or massive blood transfusions, patients who have fractured long bones (such as the femur), patients who have nearly drowned, patients who have had an adverse reaction to cancer drugs or other medications, patients who have had a drug overdose, patients with pancreatitis, patients who smoke heavily, patients who drink heavily, patients with inflammatory bowel disease, patients with rheumatoid arthritis, patients with colorectal cancer, and patients with obesity-related insulin resistance, or any combination thereof. In methods of the invention, a composition or compound may be administered directly to endothelial cells of the patient. In some cases, the endothelial cells are located near or at the lungs.

Additional methods of the invention preventing or treating a vascular permeability disease or condition comprising administering to a patient an effective amount of an HABP2 inhibitor. In further embodiments, there are methods for inhibiting angiogenesis comprising administering to a patient an effective amount of an HABP2 inhibitor.

Inhibitors include those that inhibit HABP2 activity, which includes hyaluronic acid binding, serine protease activity, and/or binding to C1NH. In some embodiments HABP2 inhibitors work by inhibiting HABP2 expression. Embodiments of the invention may involve an inhibitor that is a nucleic acid, polypeptide, or small molecule.

In specific embodiments, an HABP2 siRNA molecule is an HABP2 inhibitor. In other embodiments, an HABP2 inhibitor is an antibody that specifically recognizes HABP2 or an HABP2 binding molecule, such as C1NH or hyaluronic acid. Antibodies of the invention include those that inhibit HABP2 activity. Embodiments concern monoclonal antibodies, polyclonal antibodies, neutralizing antibodies, single-chain antibodies, humanized antibodies, chimeric antibodies, and/or antibody mimetics. A peptide construct or mimic of the polyanion binding domain of HABP2 and an inhibitor of the serine protease catalytic domain of HABP2 are other embodiments of inhibitors. High molecular weight hyaluronan directly binds and inhibits the enzymatic activity of HABP2.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is contemplated that one or more items in any list provided in the disclosure may be specifically excluded as an embodiment of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Characterization of Caveolin-enriched Microdomains and CD44 Expression in Human Pulmonary EC. FIG. 1-A: EC were grown to confluency, then serum starved for one hour. Triton X-100 soluble, Triton X-100 insoluble and Optiprep™ fractions were then prepared as described in the Materials and Methods of Example 1. The 20% Optiprep™ fraction represents the caveolin-enriched microdomain (CEM, lipid raft) fraction. The fractions were subjected to SDS-PAGE, transferred to nitrocellulose and immunoblotted with anti-Caveolin-1 (A-a), anti-Flotillin-1 (A-b), anti-Lamin A/C (A-c), anti-GRP75 (A-d), anti-GRP78 (A-e), anti-GRASP65 (A-f), anti-VEGF receptor (A-g) or anti-Vimentin (A-h) antibody. FIG. 1-B: EC were grown to confluency, then serum starved for one hour. Triton X-100 soluble, Triton X-100 insoluble and Optiprep™ fractions were then prepared as described in the Materials and Methods of Example 1. The 20% Optiprep™ fraction represents the caveolin-enriched microdomain (CEM, lipid raft) fraction. The fractions were analyzed for cholesterol content as described in Materials and Methods of Example 1. FIG. 1-C: Immunoblot analysis of EC lysates with anti-CD44 (IM-7, common domain) antibody, anti-CD44var(v3-v10) antibody, anti-CD44v3 antibody, anti-CD44v6 antibody or anti-CD44v10 antibody indicating the presence of CD44 (standard form) and CD44v10 immunoreactive bands. FIG. 1-D: RT-PCR analysis using total CD44 and CD44v10-specific primers on total RNA isolated from human EC as described in Materials and Methods of Example 1. The presence of CD44s and CD44v10 RNA are indicated by arrows.

FIG. 2. Characterization of Low and High MW Hyaluronan (HA)-induced CD44-mediated Regulation of Human EC Permeability. FIG. 2-A: EC were plated on gold microelectrodes, serum starved for one hour and either untreated (control) or treated with 1 nM, 10 nM or 100 nM High MW HA. The TER tracing represents pooled data±S.E. from three independent experiments as described in Materials and Methods of Example 1. The arrow indicates the time of High MW HA addition. FIG. 2-A (inset): Bar graph inset demonstrates that pretreatment of High MW HA by boiling (b) or proteinase K digestion (c) have little effect on High MW HA-induced EC TER (a). Treatment with hyaluronidase SD (d) blocked the effects of High MW HA which could be reversed by treatment of the HA with boiled (inactivated) hyaluronidase SD (e). FIG. 2-B: EC were plated on gold microelectrodes, serum starved for one hour and either untreated (control) or treated with 1 nM, 10 nM or 100 nM Low MW HA. The arrow indicates the time of Low MW HA addition. The TER tracing represents pooled data±S.E. from three independent experiments as described in Materials and Methods of Example 1. FIG. 2-B (inset): Bar graph inset demonstrates that pretreatment of Low MW HA by boiling (b) or proteinase K digestion (c) have little effect on Low MW HA-induced EC TER (a). Treatment with hyaluronidase SD (d) blocked the effects of Low MW HA which were reversed by treating the HA with boiled (inactivated) hyaluronidase SD (e). FIG. 2-C: Graphical representation of TER at 1 hour with no HA (control) (a), 1.0 μg/ml High MW HA (b), 10 μg/ml High MW HA (c), 100 μg/ml High MW HA (d), 1.0 μg/ml Low MW HA (e), 10 μg/ml Low MW HA (f) or 100 μg/ml Low MW HA (g). FIG. 2-D: EC were grown to confluency, serum starved for one hour, and were either untreated (control) or treated with 5 mM methyl-β-cyclodextrin (MβCD, a cholesterol depletion agent) for one hour. The 20% Optiprep™ fraction representing the caveolin-enriched microdomain (CEM, lipid raft) fractions were collected and analyzed for cholesterol content as described in Materials and Methods of Example 1. FIG. 2-E: Graphical representation of percent inhibition of HA-induced change in EC permeability. EC were plated on gold microelectrodes, serum starved for one hour and either treated with 100 nM High MW HA+control (rat pre-immune) IgG (10 μg/ml), 100 nM High MW HA+anti-CD44 (IM-7) antibody (10 μg/ml), 100 nM High MW HA+vehicle (PBS, pH=7.4), or 5 mM methyl-β-cyclodextrin (MβCD, a cholesterol depletion agent that abolishes CEM formation)+100 nM High MW HA, 100 nM Low MW HA+control (rat pre-immune) IgG (10 μg/ml), 100 nM low MW HA+anti-CD44 (IM-7) antibody (10 μg/ml), 100 nM Low MW HA+vehicle (PBS, pH=7.4), or 5 mM methyl-β-cyclodextrin (MβCD, a cholesterol depletion agent that abolishes CEM formation)+100 nM Low MW HA. The bar graphs represent pooled TER data±S.E. at 30 min. after addition of agonist from three independent experiments as described in Materials and Methods of Example 1.

FIG. 3. Analysis of HA-induced CD44 Isoform-specific Interaction with and Activation of S1P Receptors in Caveolin-enriched Microdomains. FIG. 3-A: EC were grown to confluency, serum starved for one hour and either untreated (control) or treated with 100 nM of low or high MW HA for 5, 15 or 30 min. and CEM (lipid raft) fractions (20% Optiprep™ layer) were then prepared as described in the Materials and Methods of Example 1. The CEM fractions were subjected to SDS-PAGE, transferred to nitrocellulose and immunoblotted with anti-Caveolin-1 (A-a), anti-CD44 (IM-7, common domain) (A-b), anti-CD44v10 (A-c), anti-S1P₁ receptor (A-d) or anti-S1P₃ receptor (A-e) antibody. Experiments were performed in triplicate with highly reproducible findings (representative data shown). FIG. 3-B: EC were grown to confluency, serum starved for one hour and either untreated (control) or treated with 100 nM of Low or High MW HA for 5, 15 or 30 min. and CEM fractions (20% Optiprep™ layer) were then prepared as described in the Materials and Methods of Example 1. The CEM fractions were solubilzed in IP buffer A (50 mM HEPES (pH 7.5), 150 mM NaCl, 20 mM MgCl₂, 1% Nonidet P-40 (NP-40), 0.4 mM Na₃VO₄, 40 mM NaF, 50 μM okadaic acid, 0.2 mM phenylmethylsulfonyl fluoride, 1:250 dilution of Calbiochem protease inhibitor mixture 3) and immunoprecipitated with either anti-S1P₁ or anti-S1P₃ receptor antibody. The resulting immunobeads were subjected to SDS-PAGE, transferred to nitrocellulose and immunoblotted with anti-CD44 (IM-7, common domain) (B-a,c), anti-S1P₁ receptor (B-b) or anti-S1P₃ receptor (B-d) antibody. Experiments were performed in triplicate with highly reproducible findings (representative data shown).

FIG. 4. CD44, S1P₁ and S1P₃ Silencing Inhibits HA-induced Endothelial Cell Barrier Function. FIG. 4-A: Immunoblot analysis of siRNA-treated or untreated human EC. Cellular lysates from untransfected (control, no siRNA), scramble siRNA (siRNA that does not target any known human mRNA), S1P₁ siRNA, S1P₃ siRNA or CD44 siRNA-transfection were analyzed using immunoblotting with anti-S1P₁ antibody (A-a), anti-S1P₃ antibody (A-b), anti-CD44 (IM-7) antibody (A-c), anti-Caveolin-1 antibody (A-d) or anti-actin antibody (A-e) as described in Materials and Methods of Example 1. Experiments were performed in triplicate each with similar results. Representative data is shown. FIG. 4-B: EC were plated on gold microelectrodes and treated with scramble siRNA (control), S1P₁ receptor siRNA, S1P₃ receptor siRNA or CD44 siRNA for 48 hours. EC were then serum starved for one hour followed by addition of 100 nM High MW HA. The arrow indicates the time of High MW HA addition. The TER tracing represents pooled data±S.E. from three independent experiments as described in Materials and Methods of Example 1. FIG. 4-C: EC were plated on gold microelectrodes and treated with scramble siRNA (control), S1P₁ receptor siRNA, S1P₃ receptor siRNA or CD44 siRNA for 48 hours. EC were then serum starved for one hour followed by addition of 100 nM Low MW HA. The arrow indicates the time of Low MW HA addition. The TER tracing represents pooled data±S.E. from three independent experiments as described in Materials and Methods of Example 1. FIG. 4-D: Bar graph demonstrates the inhibitory effects of S1P₁ receptor siRNA transfection of EC on S1P (the natural ligand for S1P₁ receptor), HGF, PDGF, VEGF, ATP and Thrombin-induced maximal change in TER (at least n=3 for each condition).

FIG. 5. Characterization of SIT Receptor Phosphorylation by AKT1, Src, ROCK1 and ROCK2. FIG. 5-A: EC were grown to confluency, serum starved for one hour and either untreated (control) or treated with 100 nM of Low or High MW HA or 1 μM S1P for 5, 15 or 30 min. and CEM fractions (20% Optiprep™ layer) were then prepared as described in the Materials and Methods of Example 1. The CEM fractions were solubilized in IP buffer B (50 mM HEPES (pH 7.5), 150 mM NaCl, 20 mM MgCl₂, 1% Triton X-100, 0.1% SDS, 0.4 mM Na₃VO₄, 40 mM NaF, 50 μM okadaic acid, 0.2 mM phenylmethylsulfonyl fluoride, 1:250 dilution of Calbiochem protease inhibitor mixture 3) and immunoprecipitated with either anti-S1P₁ or anti-S1P₃ receptor antibody. The resulting immunobeads were run on SDS-PAGE, transferred to nitrocellulose and immunoblotted with anti-phospho-tyrosine (A-a,e), anti-phospho-serine (A-b,f), anti-phospho-threonine (A-c,g), anti-S1P₁ receptor (A-d) or anti-S1P₃ receptor (A-h) antibody. Experiments were performed in triplicate with highly reproducible findings (representative data shown). FIG. 5-B: EC were grown to confluency, serum starved for one hour and either untreated (control) or treated with 100 nM of low or high MW HA for 5, 15 or 30 min. and CEM (lipid raft) fractions (20% Optiprep™ layer) were then prepared as described in the Materials and Methods of Example 1. The CEM fractions were run on SDS-PAGE, transferred to nitrocellulose and immunoblotted with anti-Phospho-tyrosine(418)-Src (B-a), anti-Src (B-b), anti-Phospho-serine(473)-AKT (B-c), anti-Phospho-threonine(308)-AKT (B-d), anti-AKT (B-e), anti-ROCK1 (B-f), anti-ROCK2 (B-g) or anti-Caveolin-1 antibody. Experiments were performed in triplicate with highly reproducible findings (representative data shown). FIG. 5-C: The in vitro S1P receptor phosphorylation reaction was carried out in 50 μl of the reaction mixture containing 40 mM Tris-HCl (pH 7.5), 2 mM EDTA, 1 mM dithiothreitol, 7 mM MgCl₂, 0.1% CHAPS, 0.1 μM calyculin A, 100 μM ATP, purified enzymes (i.e. 100 ng of recombinant active Src, ROCK1 or ROCK2) with or without immunoprecipitated S1P₁ or S1P₃ receptor obtained from human pulmonary EC that were serum-starved for one hour. After incubation for 30 min at 30° C., the reaction mixtures were boiled in SDS sample buffer and subjected to SDS-PAGE. Immunoblots were performed using anti-phospho-tyrosine (C-a,e), anti-phospho-serine (C-b,f), anti-phospho-threonine (C-c,g), anti-S1P₁ (C-d) or anti-S1P₃ (C-h) antibody.

FIG. 6. Effects of Silencing AKT1, Src, ROCK1 and ROCK2 Expression on SIT Receptor Phosphorylation and HA-mediated EC Barrier Function. FIG. 6-A: Immunoblot analysis of siRNA-treated or untreated human EC. Cellular lysates from untransfected (control, no siRNA), scramble siRNA (siRNA that does not target any known human mRNA), Src siRNA, AKT1 siRNA, ROCK1 siRNA or ROCK2 siRNA-transfection were analyzed using immunoblotting with anti-Src antibody (A-a), anti-AKT1 antibody (A-b), anti-ROCK1 antibody (A-c), anti-ROCK2 antibody (A-d) or anti-actin antibody (A-e) as described in Materials and Methods of Example 1. Experiments were performed in triplicate each with similar results. Representative data is shown. FIG. 6-B: EC were untransfected (control, no siRNA), scramble siRNA (siRNA that does not target any known human mRNA), Src siRNA, AKT1 siRNA, ROCK1 siRNA or ROCK2 siRNA-transfection, serum starved for one hour and either untreated (control) or treated with 100 nM of Low or High MW HA for 5 min. and CEM fractions (20% Optiprep™ layer) were then prepared as described in the Materials and Methods of Example 1. The CEM fractions were solubilized in IP buffer B (50 mM HEPES (pH 7.5), 150 mM NaCl, 20 mM MgCl₂, 1% Triton X-100, 0.1% SDS, 0.4 mM Na₃VO₄, 40 mM NaF, 50 μM okadaic acid, 0.2 mM phenylmethylsulfonyl fluoride, 1:250 dilution of Calbiochem protease inhibitor mixture 3) and immunoprecipitated with either anti-S1P₁ or anti-S1P₃ receptor antibody. The resulting immunobeads were run on SDS-PAGE, transferred to nitrocellulose and immunoblotted with anti-phospho-threonine (B-a,d), anti-S1P₁ receptor (B-b), anti-phospho-tyrosine (B-c) or anti-S1P₃ receptor (B-e) antibody. Experiments were performed in triplicate with highly reproducible findings (representative data shown). FIG. 6-C: Graphical representation of normalized resistance (TER) with scramble siRNA (siRNA that does not target any known human mRNA), Src siRNA, AKT1 siRNA, ROCK1 siRNA or ROCK2 siRNA treatment of EC. EC were plated on gold microelectrodes and treated with scramble siRNA (siRNA that does not target any known human mRNA), Src siRNA, AKT1 siRNA, ROCK1 siRNA or ROCK2 siRNA for 48 hours. EC were then serum starved for one hour followed by either no treatment (scramble control) or addition of 100 nM High or Low MW HA. The bar graphs represent pooled TER data±S.E. at 1 hour after agonist addition from three independent experiments as described in Materials and Methods of Example 1.

FIG. 7. S1P Receptor Regulation of HA-induced RhoA/Rac1 Signaling and EC Permeability. FIG. 7-A: EC were grown to confluency, serum starved for one hour and either untreated (control) or treated with 100 nM of Low or High MW HA for 5, 15 or 30 min. CEM fractions (20% Optiprep™ layer) were then prepared as described in the Materials and Methods of Example 1. The CEM fractions were subjected to SDS-PAGE, transferred to nitrocellulose and immunoblotted with anti-Tiam-1 (A-a), anti-p115 RhoGEF (A-b) or anti-Caveolin-1 (A-c) antibody. Experiments were performed in triplicate with highly reproducible findings (representative data shown). FIG. 7-B: —EC were treated with scramble siRNA (control), S1P₁ receptor siRNA or S1P₃ receptor for 48 hours. EC were grown to confluency, serum starved for one hour and either untreated (control) or treated with 100 nM of High (B-a) or Low (B-b) MW HA for 5, 15 or 30 min. EC were then solubilize in IP buffer A and incubated with p21-binding domain (PBD)-conjugated beads to bind activated (GTP-bound form) Rac1. The PBD bead-associated material was run on SDS-PAGE, transferred to nitrocellulose and immunoblotted with anti-Rac1 antibody. Experiments were performed in triplicate with highly reproducible findings (representative data shown). FIG. 7-C: EC were treated with scramble siRNA (control), S1P₁ receptor siRNA or S1P₃ receptor for 48 hours. EC were grown to confluency, serum starved for one hour and either untreated (control) or treated with 100 nM of High (C-a) or Low (C-b) MW HA for 5, 15 or 30 min. EC were then solubilize in IP buffer A and incubated with rho-binding domain (RBD)-conjugated beads to bind activated (GTP-bound form) RhoA. The RBD bead-associated material was run on SDS-PAGE, transferred to nitrocellulose and immunoblotted with anti-RhoA antibody. Experiments were performed in triplicate with highly reproducible findings (representative data shown). FIG. 7-D: Immunoblot analysis of siRNA-treated or untreated human EC. Cellular lysates from untransfected (control, no siRNA), scramble siRNA (siRNA that does not target any known human mRNA), RhoA siRNA or Rac1 siRNA-transfection were analyzed using immunoblotting with anti-RhoA antibody (a), anti-Rac1 antibody (b), anti-Caveolin-1 antibody (c) or anti-Actin antibody (d) as described in Materials and Methods of Example 1. Experiments were performed in triplicate each with similar results. Representative data is shown. FIG. 7-E: Graphical representation of normalized resistance (TER) with scramble, RhoA or Rac1 siRNA treatment of EC. EC were plated on gold microelectrodes and treated with scramble siRNA (control), RhoA siRNA or Rac1 siRNA for 48 hours. EC were then serum starved for one hour followed by either no treatment (scramble control) or addition of 100 nM High or Low MW HA. The bar graphs represent pooled TER data±S.E. at one hour after agonist addition from three independent experiments as described in Materials and Methods of Example 1.

FIG. 8. HA-induced EC Cortical Actin Rearrangement. EC were serum starved for one hour and either untreated (control), or treated with 100 nM High (FIG. 8-A) or Low (FIG. 8-B) MW HA for 5 or 30 min. Cells were then fixed and stained with TRITC-phalloidin (to visualize F-actin) and analyzed using fluorescent microscopy. These observations are representative of the entire cell monolayer and were reproduced in multiple independent experiments (at least n=3 for each condition).

FIG. 9. Analysis of HGF-induced c-Met Recruitment to Human EC Caveolin-enriched Microdomains (CEM). FIG. 9-A: After EC were grown to confluency, lysates were obtained and run on SDS-PAGE, then transferred to nitrocellulose and immunoblotted with anti-CD44 (IM-7, common domain), anti-CD44 variant (v3-v10), anti-CD44v3, anti-CD44v6 or anti-CD44v10 antibody. Experiments were performed in triplicate with highly reproducible findings (representative data shown). FIG. 9-B: EC were grown to confluency, then serum starved for one hour and either left untreated (control) or treated with 25 ng/ml HGF (5 min.) or treated with the lipid raft abolishing, cholesterol depletion agent, methyl-β-cyclodextrin (MβCD, 5 mM) for one hour prior to HGF treatment (25 ng/ml, 5 min.). After cellular material was solubilized in 4° C. 1% Triton X-100, soluble and insoluble fractions were obtained. The Triton X-100 insoluble fraction was overlaid with 60%, 40%, 30% and 20% Optiprep™ and centrifuged in a SW60 rotor (35,000 rpm, 12 h, 4° C.). The Triton X-100 soluble material and Optiprep™ fractions were run on SDS-PAGE, transferred to nitrocellulose and immunoblotted with anti-caveolin-1 (B-a), anti-c-Met (B-b), anti-CD44 (IM-7, common domain) (B-c), anti-CD44v10 (B-d) or anti-VEGF receptor 2 (B-e) antibody. The 20% Optiprep™ (*) fraction is the caveolin-enriched microdomain (CEM) fraction. Experiments were performed in triplicate with highly reproducible findings (representative data shown). FIG. 9-C: Graphical quantitation of immunoreactive bands from experiments are depicted in FIG. 9-B as analyzed using ImageQuant™ software (see Materials and Methods, Example 2). Percent of Total Protein in CEM on the y-axis refers to (S.A.G.V. 20% Optiprep™ immunoreactive band divided by (S.A.G.V. 20%+30%+40%+60% Optiprep™ immunoreactive band of interest+S.A.G.V. Triton X-100 insoluble material immunoreactive band of interest)) multiplied by 100.

FIG. 10. Effect of CD44v10 on HGF-induced c-Met Activation and Recruitment to CEM. EC were grown to confluency, then serum starved for one hour, and either left untreated (control) or treated with normal rabbit IgG (pre-immune, 10 μg/ml) or anti-CD44v10 antibody (10 μg/ml) followed by no treatment or treatment with HGF (25 ng/ml, 5 min.) and EC lysates or CEM (lipid raft) fractions (20% Optiprep™ layer) prepared as described in the Materials and Methods of Example 2. FIG. 10-A: EC lysates were run on SDS-PAGE, transferred to nitrocellulose and immunoblotted with anti-phospho-tyrosine^{1234/1235}-c-Met (A-a), anti-c-Met (A-b) or anti-actin (A-c) antibody. Experiments were performed in triplicate with highly reproducible findings (representative data shown). FIG. 10-B: Graphical quantitation of immunoreactive bands from experiments depicted in FIG. 10-A which were analyzed using ImageQuant™ software (see Materials and Methods of Example 2). Percent c-Met Phosphorylation on the y-axis refers to (S.A.G.V. phospho-tyrosine-1234/1235 c-Met immunoreactive band divided by S.A.G.V. c-Met immunoreactive band) multiplied by 100. FIG. 10-C: CEM (lipid raft) fractions (20% Optiprep™ layer) were run on SDS-PAGE, transferred to nitrocellulose and immunoblotted with anti-c-Met (C-a), anti-CD44 (IM-7, common domain) (C-b), anti-CD44v10 (C-c), anti-VEGF receptor 2 (C-d) or anti-caveolin-1 (C-e) antibody. Experiments were performed in triplicate with highly reproducible findings (representative data shown).

FIG. 11. HGF-induced c-Met/CD44 Interaction Analysis. EC were grown to confluency, then serum starved for one hour and either left untreated (control) or treated with 25 ng/ml HGF (5, 15 or 30 min.) and CEM (lipid raft) fractions (20% Optiprep™ layer) prepared as described in the Materials and Methods of Example 2. FIG. 11-A: The CEM fractions were run on SDS-PAGE, transferred to nitrocellulose and immunoblotted with anti-phospho-tyrosine^{1234/1235}-c-Met (A-a), anti-phospho-tyrosine¹³⁴⁹-c-Met (A-b), anti-c-Met (A-c), anti-CD44 (IM-7, common domain) (A-d), anti-CD44v10 (A-e), anti-VEGF receptor 2 (A-f) or anti-caveolin-1 (A-g) antibody. Experiments were performed in triplicate with highly reproducible findings (representative data shown). FIG. 11-B: EC lysates were solubilized in IP buffer A (50 mM HEPES (pH 7.5), 150 mM NaCl, 20 mM MgCl₂, 1% Nonidet P-40 (NP-40), 0.4 mM Na₃VO₄, 40 mM NaF, 50 μM okadaic acid, 0.2 mM phenylmethylsulfonyl fluoride, 1:250 dilution of Calbiochem protease inhibitor mixture 3) and immunoprecipitated with anti-c-Met antibody. The resulting immunobeads were run on SDS-PAGE, transferred to nitrocellulose and immunoblotted with anti-CD44 (IM-7, common domain) (B-a), anti-phospho-serine (B-b) or anti-c-Met (B-c) antibody. Experiments were performed in triplicate with highly reproducible findings (representative data shown). FIG. 11-C: The CEM fractions were solubilized in IP buffer A (see above) and immunoprecipitated with anti-CD44 (IM-7, common domain) antibody. The resulting immunobeads were run on SDS-PAGE, transferred to nitrocellulose and immunoblotted with anti-c-Met (C-a), anti-phospho-serine (C-b) or anti-CD44 (IM-7, common domain) (C-c) antibody. Experiments were performed in triplicate with highly reproducible findings (representative data shown).

FIG. 12 Effect of CEM, c-Met and CD44 on HGF-induced Human EC Barrier Enhancement. FIG. 12-A: Immunoblot analysis of siRNA-treated or untreated human EC. Cellular lysates from untransfected (control, no siRNA), scramble siRNA (siRNA that does not target any known human mRNA), c-Met siRNA or CD44 siRNA-transfection were analyzed using immunoblotting with anti-c-Met (A-a), anti-CD44 (IM-7) antibody (A-b) or anti-actin antibody (A-c) as described in Materials and Methods of Example 2. Experiments were performed in triplicate, each with similar results and representative data is shown. FIG. 12-B: EC were plated on gold microelectrodes, serum-starved for one hour and treated with either PBS, pH=7.4 (control) or 5 mM methyl-β-cyclodextrin (MβCD, a cholesterol depletion agent that abolishes CEM formation) 30 min. prior to PBS, pH=7.4 or 25 ng/ml HGF addition. The arrows indicate the times of MβCD and HGF addition. The TER tracing represents pooled data±S.E. from three independent experiments as described in Materials and Methods. FIG. 12-C: EC were plated on gold microelectrodes and treated with scramble siRNA (control) or c-Met siRNA for 48 hours. EC were then serum starved for one hour followed by addition of 25 ng/ml HGF. The arrow indicates the time of HGF addition. The TER tracing represents pooled data±S.E. from three independent experiments as described in Materials and Methods of Example 2. FIG. 12-D: EC were plated on gold microelectrodes and treated with scramble siRNA (control) or CD44 siRNA for 48 hours. EC were then serum starved for one hour followed by addition of 25 ng/ml HGF. The arrow indicates the time of HGF addition. The TER tracing represents pooled data±S.E. from three independent experiments as described in Materials and Methods of Example 2. FIG. 12-E: Graphical representation of percent maximal sphingosine 1-phosphate (S1P)-induced change in EC permeability. EC were plated on gold microelectrodes and treated with no siRNA, scramble siRNA, CD44 siRNA or S1P₁ receptor siRNA for 48 hours. EC were then serum starved for one hour followed by addition of 1 μM S1P. The bar graphs represent pooled TER data±S.E. at 30 min. after addition of agonist from three independent experiments as described in Materials and Methods of Example 2.

FIG. 13. Role of CD44 in HGF-induced Recruitment of c-Met, Tiam1, Cortactin and Dynamin 2 to Human EC CEM. EC were treated with scramble siRNA (control) or CD44 siRNA for 48 hours. EC were then grown to confluency, serum starved for one hour and either untreated (control) or treated with 25 ng/ml HGF for 5, 15 or 30 min. FIG. 13-A: EC lysates were run on SDS-PAGE, transferred to nitrocellulose and immunoblotted with anti-phospho-tyrosine^{1234/1235}-c-Met (a,c), anti-c-Met (b,d) antibody. Experiments were performed in triplicate with highly reproducible findings (representative data shown). FIG. 13-B: CEM (lipid raft) fractions (20% Optiprep™ layer), prepared as described in the Materials and Methods of Example 2, were run on SDS-PAGE, transferred to nitrocellulose and immunoblotted with anti-c-Met (a,f), anti-anti-Tiam1 (b,g), anti-cortactin (c,h), anti-dynamin 2 (d,i) or anti-caveolin-1 (e,j) antibody. Experiments were performed in triplicate with highly reproducible findings (representative data shown).

FIG. 14. Effect of Tiam1, Cortactin and Dynamin 2 on HGF-induced Human EC Barrier Enhancement. FIG. 14-A: Immunoblot analysis of siRNA-treated or untreated human EC. Cellular lysates from untransfected (control, no siRNA), scramble siRNA (siRNA that does not target any known human mRNA), Tiam1 siRNA, dynamin 2 siRNA or cortactin siRNA-transfection were analyzed using immunoblotting with anti-Tiam1 (A-a), Anti-dynamin 2 (A-b), anti-cortactin (A-c) or anti-actin (A-d) antibody as described in Materials and Methods of Example 2. Experiments were performed in triplicate each with similar results. Representative data is shown. For FIG. 14-B and -C, EC were then grown to confluency, serum starved for one hour and either untreated (control) or treated with 25 ng/ml HGF for 5, 15 or 30 min. and CEM (lipid raft) fractions (20% Optiprep™ layer) were then prepared as described in the Materials and Methods of Example 2. FIG. 14-B: EC were treated with scramble siRNA (control) dynamin 2 siRNA or Tiam1 siRNA for 48 hours. The CEM fractions were run on SDS-PAGE, transferred to nitrocellulose and immunoblotted with anti-cortactin (B-a,c,e) or anti-caveolin-1 (B-b,d,f) antibody. Experiments were performed in triplicate with highly reproducible findings (representative data shown). FIG. 14-C: CEM fractions were solubilized in IP buffer A (50 mM HEPES (pH 7.5), 150 mM NaCl, 20 mM MgCl₂, 1% Nonidet P-40 (NP-40), 0.4 mM Na₃VO₄, 40 mM NaF, 50 μM okadaic acid, 0.2 mM phenylmethylsulfonyl fluoride, 1:250 dilution of Calbiochem protease inhibitor mixture 3) and immunoprecipitated with anti-dynamin 2 antibody. The resulting immunobeads were run on SDS-PAGE, transferred to nitrocellulose and immunoblotted with anti-Tiam1 (C-a), anti-cortactin (C-b), anti-caveolin-1 (C-c) or anti-dynamin 2 (C-d) antibody. Experiments were performed in triplicate with highly reproducible findings (representative data shown). FIG. 14-D: Graphical quantitation of immunoreactive bands from experiments depicted in Panel C which were analyzed using ImageQuant™ software (see Materials and Methods of Example 2). % Protein Association with Dynamin 2 on the y-axis refers to (S.A.G.V. immunoreactive band of interest divided by S.A.G.V. dynamin 2 immunoreactive band) multiplied by 100. FIG. 14-E: Graphical representation of percent maximal HGF-induced change in EC permeability. EC were plated on gold microelectrodes and treated with scramble siRNA (control), Tiam1 siRNA, dynamin 2 siRNA or cortactin siRNA for 48 hours. EC were then serum starved for one hour followed by addition of 25 ng/ml HGF. The bar graphs represent pooled TER data±S.E. at 30 min. after addition of agonist from three independent experiments as described in Materials and Methods of Example 2.

FIG. 15. The Effect of Tiam1, Cortactin and Dynamin 2 on HGF-induced Rac1 Activation. FIG. 15-A: EC were either untreated, treated with scramble siRNA, c-Met siRNA, CD44 siRNA, dynamin 2 siRNA, Tiam1 siRNA or cortactin siRNA for 48 hours. EC were grown to confluency, serum starved for one hour and either untreated (control) or treated with 5 mM methyl-P-cyclodextrin (MβCD, a cholesterol depletion agent that abolishes CEM formation) 30 min. prior to PBS, pH=7.4 or 25 ng/ml HGF addition. EC were then solubilized in IP buffer and incubated with p21-binding domain (PBD)-conjugated beads to bind activated (GTP-bound form) Rac1. The PBD bead-associated material was run on SDS-PAGE, transferred to nitrocellulose and immunoblotted with anti-Rac1 antibody. Experiments were performed in triplicate with highly reproducible findings (representative data shown). FIG. 15-B: Graphical quantitation of immunoreactive bands from experiments depicted in Panel A which were analyzed using ImageQuant™ software (see Materials and Methods of Example 2). % Rac1 Activation on the y-axis refers to (S.A.G.V. activated Rac1 immunoreactive band divided by S.A.G.V. total Rac1 immunoreactive band) multiplied by 100. FIG. 15-C: Immunoblot analysis of siRNA-treated or untreated human EC. Cellular lysates from untransfected (control, no siRNA), scramble siRNA (siRNA that does not target any known human mRNA) or Rac1 siRNA-transfection were analyzed using immunoblotting with anti-Rac1 (C-a) or anti-actin antibody (C-b) as described in Materials and Methods of Example 2. Experiments were performed in triplicate each with similar results. Representative data is shown. FIG. 15-D: Graphical representation of percent maximal HGF-induced change in EC permeability. EC were plated on gold microelectrodes and treated with scramble siRNA (control) or Rac1 siRNA for 48 hours. EC were then serum starved for one hour followed by addition of 25 ng/ml HGF. The bar graphs represent pooled TER data±S.E. at 30 min. after addition of agonist from three independent experiments as described in Materials and Methods of Example 2.

FIG. 16. Role of CD44 on HGF-induced Protection from LPS-induced Vascular Hypermeability in vivo. FIG. 16-A: Immunohistochemical fluorescent staining images of control (untreated) mouse lung using either bright field (DIC) imaging (a) or treatment with anti-Factor VIII (vWF) antibody (b), anti-c-Met antibody (c) or FITC-conjugated anti-CD44 antibody (d) and secondary fluorescent antibody (Alexa Fluor™ 610 (for vWF) and 350 (for c-Met), (Molecular Probes) as described in Materials and Methods of Example 2. Images are shown at 100× magnification. Arrows indicate immunostaining of endothelial cells with (e) being an overlay of (b, c and d). FIG. 16-A (insets): Negative controls for immunohistochemical analysis which were done by the same method as above but without primary antibody. FIGS. 16-B and -C: Male C57BL/6J and CD44 knockout mice were anesthetized and were either given saline (control) or LPS (2.5 mg/kg) intratracheally. After 4 hours, mice were given internal jugular vein intravenous injections with saline (control) or high molecular weight hyaluronan (HMW-HA, 1.5 mg/kg) (B) or HGF (50 μg/kg) (C). The treated mice were allowed to recover for 24 hours. Bronchioalveolar lavage (BAL) fluids were then obtained and protein concentrations were determined (see Materials and Methods of Example 2). For FIGS. 16-B and -C, the single asterisk (*) refers to a significant (p<0.05) difference between control and LPS treatment. There is also a significant difference (p<0.05) between LPS and HMW-HA+LPS treatment in the wildtype, but not the CD44 knockout, mouse. FIG. 16-C: The double asterisk (**) refers to a significant difference (p<0.05) between LPS treatment and HGF+LPS treatment. There is also a significant difference (p<0.05) between the wildtype and CD44 knockout mouse HGF+LPS treatment.

FIG. 17 HABP2 regulates hyaluronan- and LPS-induced EC barrier function. High MW hyaluronan increases transendothelial monolayer electrical resistance (TER), whereas low MW hyaluronan and LPS induce negative TER changes ultimately resulting in EC barrier disruption. Silencing of HABP2 expression promoted the EC barrier enhancing effects of high MW hyaluronan and consistently overexpression of HABP2 blocked these effects. HABP2 silencing also reduced the effects of low MW hyaluronan and LPS on EC barrier disruption while HABP2 overexpression enhanced these effects.

FIG. 18 Male C57BL/6J, CD44 knockout and Caveolin-1 knockout mice were anesthetized and were either given saline (control) or LPS (2.5 mg/kg) intratracheally. After 4 hours, mice were given intravenously injections (internal jugular vein) with saline (control) or high molecular weight hyaluronan (HMW-HA, 1.5 mg/kg). The treated mice were allowed to recover for 24 hours, bronchioalveolar lavage (BAL) fluids were obtained and concentrations of total protein (A), TGF-alpha (B), TGF-beta1 (C) were determined. N=6 per condition with the single asterisk (*) referring to a significant (p<0.05) difference between control and LPS treatment. High MW hyaluronan reduced the enhancing effect of LPS on BAL protein concentration and also TGF-alpha and TGF-beta1 concentration in BAL fluids of wild type mice, but not in CD44 knockout and Caveolin-1 knockout mice.

FIG. 19 Inhibition of maximal high MW TER response. The effect of siRNA silencing of CD44, Caveolin-1, Tiam1, Dynamin2, Rac1 or the P13 kinase inhibitor, LY294002 (10 μM) was compared to control or scramble siRNA on inhibition of HMW-HA-induced TER response.

FIG. 20 Inhibition of LPS-mediated EC barrier disruption at 6 hours in the presence or absence of No siRNA, HMW-HA (100 nM)+No siRNA, Scramble siRNA, RhoA siRNA, ROCK 1/2 siRNA, MARCKS siRNA or the NHE1 inhibitor (4-Cyanobenzo[b]thiophen-2-carbonyl) guanidine,methanesulfonate (10 μM).

FIG. 21 Analysis of HABP2 expression and hyaluronan regulation of purified HABP2 activity. Panel A—EC were transfected with an HABP2 overexpression vector for 48 hours, media was collected and immunoprecipitated with anti-HABP2 antibody covalently linked to sepharose beads. The bound HABP2 was eluted and protease activity assays were performed in the presence of various concentrations of either HMW-HA or LWM-HA as described in the Experimental Design and Methods. Panel B—Similar to Panel A, protease activity assays were performed on purified HABP2 in the presence or absence of 500 nM HMW-HA or 500 nM LMW-HA with or without 100 μM purified PABD as described in the Methods.

FIG. 22 Analysis of HABP2 effects on EC barrier function and proteolytic targets. Panel A—Graphical representation of TER measurements as described in Panel A were obtained from EC transfected with scramble siRNA (control), PAR-1 siRNA, PAR-2 siRNA, PAR-3 siRNA or PAR-4 siRNA for 48 hours followed by addition of 10 μg/ml purified HABP2 or 1 Unit/ml thrombin. The y-axis indicates % maximal change in TER with N=3 per condition. Panel B—Graphical representation of TER measurements as described in Panel A were obtained from EC transfected with scramble siRNA (control), tenascin-C siRNA or perlecan siRNA for 48 hours followed by basal TER measurements. The y-axis indicates % basal TER with N=3 per condition.

FIG. 23 Analysis of C1INH expression and regulation of EC barrier function. Panel A—EC were grown to confluency, media were collected, concentrated and immunoprecipitated with anti-HABP2 antibody-conjugated Sepharose beads. The HABP2-bound beads were eluted, run on non-reducing SDS-PAGE and immunoblotted with anti-HABP2 or anti-C1INH antibody. Panel B—EC were grown to confluency on ECIS plates and Transendothelial Resistance (TER) measurements were obtained with no treatment (control) or addition of either 1.0 μg/ml LPS, 100 nM HMW-HA, 100 nM LMW-HA or 10 μg/ml purified HABP2. The resulting graph represents data from three experiments. The y-axis indicates % maximal change in TER.

FIG. 24 Analysis of HABP2 and C1INH expression and complex formation in murine lungs with or without LPS treatment and effective silencing of HABP2 expression in murine lungs. Panel A—Male B6129N2 mice (8-10 weeks) were anesthetized with intraperitoneal ketamine (150 mg/kg) and acetylpromazine (15 mg/kg) before exposure of the right internal jugular vein via neck incision. LPS (2.5 mg/kg) or water (control) were instilled intravenously through the internal jugular vein. The animals were allowed to recover for 24 hours after LPS before lung extraction. Extracted lungs were homogenized, N=6 samples per condition were pooled, run on SDS-PAGE and immunoblotted with anti-HABP2 (1), anti-C1INH (2) or anti-actin (3) antibody. Panel B—Homogenized lung samples as described in Panel A were immunoprecipitated with anti-HABP2 antibody, run on non-reducing SDS-PAGE and immunoblotted with either anti-HABP2 or anti-C1INH antibody. The upper arrow indicates an SDS-stable complex between HABP2 and C1INH. The lower arrow indicates the free (active) faun of HABP2. Panel C—Male B6129N2 mice (8-10 weeks) were anesthetized with intraperitoneal ketamine (150 mg/kg) and acetylpromazine (15 mg/kg) before exposure of the right internal jugular vein via neck incision. 10 mg/kg in vivo stable scramble siRNA (control) or HABP2 siRNA (Dharmacon) were instilled intravenously through the internal jugular vein. The animals were allowed to recover for either 72 or 120 hours after siRNA delivery before lung extraction. Extracted lungs were homogenized, N=2 samples per condition were pooled, run on SDS-PAGE and immunoblotted with anti-HABP2 (1) or anti-actin (2) antibody.

FIG. 25 Analysis of CD44 isoform and hyaluronidase expression and HA effects on VEGF-induced angiogenic events. Panels A and B—EC were treated with 100 nM VEGF, HMW-HA (−1 million Daltons), VEGF+HMW-HA, LMW-HA (approximately 2,500 Daltons) or LMW-HA+VEGF and analyzed for % Migration (A) or % Proliferation (B) as described in the Methods. Panel C—Demonstration of successful EC tube formation using VEGF (100 nM)-embedded matrigel.

FIG. 26 Analysis of HABP2 and C1INH expression, HA regulation of HABP2 activity and HABP2 regulation of VEGF-induced angiogenic events. Panel A—EC were treated with 100 nM VEGF, HMW-HA (approximately 1 million Daltons), VEGF+HMW-HA, LMW-HA (˜2,500 Daltons) or LMW-HA+VEGF, lysates obtained, run on SDS-PAGE and immunoblotted with anti-HABP2 (1) or anti-actin (2) antibody. Panel B—EC were transfected with vector control or HABP2 overexpression vectors for 48 hours. Then, media were collected and immunoprecipitated with anti-HABP2 antibody-conjugated Sepharose beads. Protease activity assays were performed on the immunoprecipitated material in the presence of various concentrations of either HMW-HA or LWM-HA as described in the Experimental Design and Methods. Panel C—EC were treated with 100 nM VEGF, HMW-HA or VEGF+HMW-HA, lysates obtained, run on SDS-PAGE and immunoblotted with anti-C1INH (1) or anti-actin (2) antibody. Panel D—EC were treated with either scramble siRNA or HABP2 siRNA for 48 hours, lysates obtained, run on SDS-PAGE and immunoblotted with anti-HABP2 (1) or anti-actin (2) antibody. Panels E and F—EC were treated with either scramble siRNA or HABP2 siRNA for 48 hours and analyzed for VEGF-induced % Migration (C) or % Proliferation (D) as described in the Methods.

FIG. 27A-B Inhibition of HAPB2 in vivo. Panel A. Homogenized lungs (a,b) and plasma (c,d) were probed with either anti-HABP2 (a,c), anti-actin (b) or anti-fibronectin (d) antibodies followed by specific secondary antibodies. The results indicate successful inhibition of HABP2 protein expression with HABP2 siSTABLE siRNA in mouse lung and serum. Panel B. Graph showing extent of protection in mice with LPS-induced ALI. The y-axis indicates the concentration of BAL protein (mg/ml) for each pooled N=5 sample. The single asterisk (*) refers to a significant (p<0.05) difference between control and LPS treatment. There is also a significant difference (p<0.05) between control (no siRNA)+LPS and HABP2 siSTABLE siRNA+LPS treatment indicating silencing HABP2 protein expression protects mice from LPS-induced ALI.

### DETAILED DESCRIPTION OF THE INVENTION

The inner lining of all blood vessels is comprised of endothelial cells (EC), which regulate the interface between the blood and the vessel wall including vascular barrier regulation, passive diffusion and active transport of substances from the blood, regulation of vascular smooth muscle tone and blood clotting (Pearson, 1991; Luscher et al., 1997). Disruption of this semi-selective cellular barrier is a significant feature of inflammation, in addition to being a crucial contributing factor to atherosclerosis and tumor angiogenesis (Dudek et al., 2001; Garcia et al., 2001). Several bioactive agonists contribute to EC barrier regulation via direct effects on the integrity of EC junctions, cell-cell and cell-matrix adhesions. While previous reports have implicated one important extracellular matrix component, hyaluronan (HA), and its cell surface receptor, CD44 (Turley et al., 2002; Toole, 2004), the Examples provide evidence that these molecules are involved in normal EC function and angiogenesis.

### I. HYALURONAN

Hyaluronan (HA) is a major glycosaminoglycan (GAG) component of the extracellular matrix of many tissues. Structurally, high molecular weight (HMW) HA (approximately 1 million daltons or more) is composed of repeating disaccharide units of D-glucuronic acid and N-acetylglucosamine which exists as a random coil structure that can expand in aqueous solutions (Toole, 2004; Scott et al., 2002). Aqueous HA is highly viscous and elastic, properties which contribute to its space filling and filtering functions (Scott et al., 2002). Proinflammatory cytokines (TNFα, IL-1β) and LPS induce HA production in EC in vitro (Mohamadzadeh et al., 1998) and increased HA levels are observed in bronchioalveolar lavage fluid (BALF) from patients with inflammatory lung disorders such as pulmonary fibrosis, acute lung injury, and chronic obstructive pulmonary disease (Bensadoun et al., 1996; Dentener et al., 2005; Nettelbladt et al., 1989; Teder et al., 1997). Intratracheal administration of nebulized high MW HA has been used to prevent injury in experimental emphysema (Cantor et al., 2004). Further, HA and CD44 regulate IL2-induced vascular injury syndrome in mouse lung (Mustafa et al., 2002; Rafi-Janajreh et al., 1999).

HA is degraded by hyaluronidases, under certain pathological inflammatory conditions, to produce lower molecular weight fragments found in tissue injury and serum of patients with certain malignancies (Orian-Rousseau et al., 2002; Orian-Rousseau et al., 2007). Further, low MW fragments of HA (LMW, 1,350-4,500 Da) are potent inducers of angiogenesis in vitro and in vivo (Lokeshwar et al., 1996; Hirano et al., 1994). Six hyaluronidase genes encode Hyal-1, 2, 3, 4, PHYAL1 (a pseudogene) and PH-20 with high MW HA and its fragments binding hyaladherin proteins including CD44, a major HA receptor (Liu et al., 2002; Ishizawa et al., 2004).

Hyaluronan binds to the hyaladherin family of transmembrane glycoproteins (including CD44) which are expressed in a variety of cells including EC (Singleton et al., 2004; Singleton et al., 2002). Multiple CD44 isoforms result from extensive, alternative exon splicing events (Lokeshwar et al., 1996; Hirano et al., 1994) with the alternative splicing often occurring between exons 5 and 15 leading to a tandem insertion of one or more variant exons (v1-v10, or exons 6 through exons 14 in human cells) within the membrane proximal region of the extracellular domain (Gee et al., 2004; Bourguignon et al., 1998). The variable primary amino acid sequence of different CD44 isoforms is further modified by extensive N- and O-glycosylations and glycosaminoglycan (GAG) additions (Turley et al., 2002; Bourguignon et al., 1998). The extracellular domain of CD44, containing clusters of conserved basic residues, plays an important role in HA binding, whereas the cytoplasmic domain is both structurally and functionally linked to cytoskeletal elements and signaling molecules (Turley et al., 2002; Bourguignon et al., 1998). The signaling properties of CD44 are required for a variety of cellular activities including EC adhesion, proliferation, migration and angiogenesis (Turley et al., 2002; Singleton et al., 2004; Singleton et al., 2002; Bourguignon et al., 1998; Toole et al., 2002). Further, CD44−/− mice develop lung fibrosis, inflammatory cell recruitment and accumulation of hyaluronan fragments at sites of lung injury (Teder et al., 2002).

Hyaluronan can be obtained from rooster comb, human umbilical cord, and bovine organs such as trachea. It is also available commercially from Annika Therapeutics, Inc. (see World Wide Web at fda.gov/cdrh/pdf3/p030019c.pdf), Biomatrix, ICN, and Pharmacia. HA can also been produced using bacterial fermentation, such as with streptococcal bacteria.

Hyaluronan can also be reacted in a number of schemes, such as those described in US 2002/0086852, which is hereby incorporated by reference in its entirety.

### II. HABP2

Hyaluronic Acid Binding Protease 2 (HABP2) is an extracellular serine protease highly expressed in lungs (Wygrecka et al., 2007a). HABP2 contains 3 EGF-like domains, a kringle-like domain and a trypsin-like protease domain (Romisch, 2002; Kannemeier et al. 2001). The polyanion binding domain (PABD) is contained within the second and third EGF-like domains (Altinicicek et al., 2006).

HABP2, has been implicated in regulating acute lung injury (ALI) however the mechanism by which this occurs is unknown (Wygrecka et al 2007a; Wygrecka et al., 2007b). HABP2 protein expression and activity are upregulated in the lungs of acute respiratory distress syndrome (ARDS) patients (Wygrecka et al., 2007a). Further, HABP2, also called factor VII activating protease, is involved in regulating the blood coagulation cascade through cleavage of factor VII, pro-urokinase type plasminogen activator (uPA), fibrinogen and kininogen (Romisch, 2002; Kannemeier et al. 2001).

The level of HABP2 expression in the cell is measured in screening methods of the invention. In other embodiments, HABP2 expression is measured by measuring the amount of HABP2 protein in the cell. In particular embodiments, HABP2 activity is measured. HABP2 has serine protease activity and it has the ability to form a complex with C1NH. In certain embodiments, HABP2 activity is measured by fibrinolysis assay, coagulation assay, protease assay, surface plasmon resonance binding assay, fluorescence polarization, radioactive tracer assay, and/or homogeous time-resolved fluorescence assay (Zbikowska et al. 2007; Demple, 1999; Blomback, 1994; Felmeden et al., 2005; Ware et al., 2005, all of which are hereby incorporated by reference). In specific embodiments, HABP2 complex formation with C1 NH is used to evaluate HABP2 activity, such as by detecting a loss of binding to or complex formation with C1NH.

A. HABP2 Inhibitors

Methods of the invention involve administering or prescribing an HABP2 inhibitor or screening for HABP2 inhibitors. Inhibitors of HABP2 activity or expression include nucleic acids, polypeptides, or small molecules. In certain embodiments, nucleic acid inhibitors include those with sequences complementary or identical to an HABP2-encoding sequence. In other embodiments, inhibitors of HABP2 activity or expression include polypeptides, such as molecules with antibody or antibody-like activity in their ability to specifically recognize and bind HABP2 or those that mimic the polyanion binding domain of HABP2.

1. Antisense Sequences, Including siRNAs

In particular embodiments, the invention concerns isolated nucleic acid segments and recombinant vectors incorporating DNA sequences that encode HABP2 inhibitors, such as HABP2 siRNAs, ribozymes and HABP2 antibodies and other HABP2 binding proteins or proteins that inhibit expression of HABP2 transcipts.

In some embodiments, a nucleic acid may encode an antisense construct. Antisense methodology takes advantage of the fact that nucleic acids tend to pair with “complementary sequences.” By complementary, it is meant that polynucleotides are those which are capable of base-pairing according to the standard Watson-Crick complementarity rules. Inclusion of less common bases such as inosine, 5-methylcytosine, 6-methyladenine, hypoxanthine and others in hybridizing sequences does not interfere with pairing.

Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

In certain embodiments, the nucleic acid encodes an interfering RNA or siRNA. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery, 1999; Montgomery et al., 1998; Sharp and Zamore, 2000; Tabara et al., 1999). Activation of these mechanisms targets mature, dsRNA-complementary mRNA for destruction. Advantages of RNAi include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs are designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 29 nucleotides in length are most effective. This length reflects the lengths of digestion products resulting from the processing of much longer RNAs as described above (Montgomery et al., 1998). siRNAs to HABP2 are commerically available such as the HuSH 29-mer shRNA construct against HABP2 from Origene (cat. #TR312532).

The making of siRNAs has been mainly through direct chemical synthesis; or through an in vitro system derived from S2 cells. Chemical synthesis proceeds by making two single stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

In some embodiments, the invention concerns an siRNA that is capable of triggering RNA interference, a process by which a particular RNA sequence is destroyed. siRNA are dsRNA molecules that are 100 bases or fewer in length (or have 100 basepairs or fewer in its complementarity region). In some cases, it has a 2 nucleotide 3′ overhang and a 5′ phosphate. The particular RNA sequence is targeted as a result of the complementarity between the dsRNA and the particular RNA sequence. It will be understood that dsRNA or siRNA of the invention can effect at least a 20, 30, 40, 50, 60, 70, 80, 90 percent or more reduction of expression of a targeted RNA in a cell. dsRNA of the invention (the term “dsRNA” will be understood to include “siRNA”) is distinct and distinguishable from antisense and ribozyme molecules by virtue of the ability to trigger RNAi. Structurally, dsRNA molecules for RNAi differ from antisense and ribozyme molecules in that dsRNA has at least one region of complementarity within the RNA molecule. The complementary (also referred to as “complementarity”) region comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous bases, or any range derivable therein, to sequences (or their complements) disclosed herein, including HABP2. In some embodiments, the sequence is complementary or identical to all or any portion of contiguous nucleic acid molecules described in this paragraph of SEQ ID NO:1 or SEQ ID NOs:3-36. SEQ ID NO:1 is the cDNA sequence for human HABP2 (Genbank Accession number NM_{—}004132, which is hereby incorporated by reference). SEQ ID NO:2 is the encoded polypeptide.

In some embodiments, long dsRNA are employed in which “long” refers to dsRNA that are 1000 bases or longer (or 1000 basepairs or longer in complementarity region). The term “dsRNA” includes “long dsRNA” and “intermediate dsRNA” unless otherwise indicated. In some embodiments of the invention, dsRNA can exclude the use of siRNA, long dsRNA, and/or “intermediate” dsRNA (lengths of 100 to 1000 bases or basepairs in complementarity region). It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (see Sui et al., 2002 and Brummelkamp et al., 2002 in which a single strand with a hairpin loop is used as a dsRNA for RNAi). For convenience, lengths of dsRNA may be referred to in terms of bases, which simply refers to the length of a single strand or in terms of basepairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

The single RNA strand or two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 31, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more (including the full-length of a particular's gene's mRNA without the poly-A tail) bases or basepairs. If the dsRNA is composed of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

In some embodiments, the strand or strands of dsRNA are 100 bases (or basepairs) or less, in which case they may also be referred to as “siRNA.” In specific embodiments the strand or strands of the dsRNA are less than 70 bases in length. With respect to those embodiments, the dsRNA strand or strands may be from 5-70, 10-65, 20-60, 30-55, 40-50 bases or basepairs in length. A dsRNA that has a complementarity region equal to or less than 30 basepairs (such as a single stranded hairpin RNA in which the stem or complementary portion is less than or equal to 30 basepairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 basepairs or fewer. In some cases, a dsRNA may be processed in the cell into siRNA.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

PCT publications WO 99/32619 and WO 01/68836 suggest that RNA for use in siRNA may be chemically or enzymatically synthesized. Both of these texts are incorporated herein in their entirety by reference. The contemplated constructs provide templates that produce RNAs that contain nucleotide sequences identical to a portion of the target gene. Typically the length of identical sequences provided is at least 25 bases, and may be as many as 400 or more bases in length. Longer dsRNAs may be digested to 21-25mer lengths with endogenous nuclease complex that converts long dsRNAs to siRNAs in vivo. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. U.S. Pat. No. 5,795,715 reports the simultaneous transcription of two complementary DNA sequence strands in a single reaction mixture, wherein the two transcripts are immediately hybridized.

2. Polypeptides and Peptides

In particular embodiments, the invention concerns HABP2 inhibitors that are polypeptides or peptides. In some embodiments, such inhibitors may bind to HABP2 or may mimic HAPB2.

a. Protein Mimics

Some embodiments of the present invention pertain to HABP2 inhibitors that mimic HABP2, whose polypeptide sequence is disclosed in NM_{—}004132 or SEQ ID NO:2. In certain embodiments, an inhibitor comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, or 560 contiguous amino acids of SEQ ID NO:2 or any fragment discussed herein, or any range derivable therein. Alternatively, any polypeptide inhibitors may have, have at least, or have at most 60%, 65%, 70%, 75%, 80%, 85%, 90%, 95%, 96%, 97%, 98%, 99%, 99.5%, 99.6%, 99.7%, 99.8%, 99.9% identity with SEQ ID NO:2 or the fragments discussed herein, or any combination thereof. Alternatively, these characteristics of identity with SEQ ID NO:2 may be combined with the characteristic of contiguous amino acid lengths of SEQ ID NO:2 to describe inhibitors contemplated by the present invention.

In some embodiments of the invention the inhibitor is a mimic of the HABP2 polyanion binding domain, which is amino acids 110-188 of Genbank Accession number (human, GI:73919921, which is hereby incorporated by reference) or SEQ ID NO:2: KVQNTCKDNPCGRGQCLITQSPPYYRCVCKHPYTGPSCSQVVPVCRPNPCQNGATCS RHKRRSKFTCACPDQFKGKFCE (SEQ ID NO:37).

In other embodiments, the inhibitor may mimic the HABP2 serine protease catalytic domain, which is amino acids 314-555 of human, GI:73919921 or SEQ ID NO:2: YGGFKSTAGKHPWQASLQSSLPLTISMPQGHFCGGALIHPCWVLTAAHCTDIKTRHL KVVLGDQDLKKEEFHEQSFRVEKIFKYSHYNERDEIPHNDIALLKLKPVDGHCALES KYVKTVCLPDGSFPSGSECHISGWGVTETGKGSRQLLDAKVKLIANTLCNSRQLYDH MIDDSMICAGNLQKPGQDTCQGDSGGPLTCEKDGTYYVYGIVSWGLECGKRPGVYT QVTKFLNWIKATIK (SEQ ID NO:38).

b. Antibody Production

Some embodiments of the present invention pertain to methods and compositions involving an inhibitor of HABP2, wherein the inhibitor is an antibody that binds HABP2.

As used herein, the term “antibody” refers to any form of antibody or fragment thereof that exhibits the desired biological activity. Thus, it is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. An antibody inhibitor may be considered a neutralizing antibody.

Included within the definition of an antibody that binds HABP2 is a HABP2 antibody binding fragment. As used herein, the term “HABP2 binding fragment” or “binding fragment thereof” encompasses a fragment or a derivative of an antibody that still substantially retain its biological activity of inhibiting HABP2 activity. Therefore, the term “antibody fragment” or HABP2 binding fragment refers to a portion of a full length antibody, generally the antigen binding or variable region thereof. Examples of antibody fragments include Fab, Fab′, F(ab′).sub.2, and Fv fragments; diabodies; linear antibodies; single-chain antibody molecules, e.g., sc-Fv; and multispecific antibodies formed from antibody fragments. Typically, a binding fragment or derivative retains at least 50% of its HABP2 inhibitory activity. Preferably, a binding fragment or derivative retains about or at least about 60%, 70%, 80%, 90%, 95%, 99% or 100% of its HABP2 inhibitory activity. It is also intended that a HABP2 binding fragment can include conservative amino acid substitutions that do not substantially alter its biologic activity.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975), or may be made by recombinant DNA methods (see, e.g., U.S. Pat. No. 4,816,567). The “monoclonal antibodies” may also be isolated from phage antibody libraries using the techniques described in Clackson et al. (1991) and Marks et al. (1991), for example.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Any suitable method for generating monoclonal antibodies may be used. For example, a recipient may be immunized with HABP2 or a fragment thereof. Any suitable method of immunization can be used. Such methods can include adjuvants, other immunostimulants, repeated booster immunizations, and the use of one or more immunization routes.

Any suitable source of HABP2 can be used as the immunogen for the generation of the non-human antibody of the compositions and methods disclosed herein. Such forms include, but are not limited whole protein, peptide(s), and epitopes, generated through recombinant, synthetic, chemical or enzymatic degradation means known in the art.

Any form of the antigen can be used to generate the antibody that is sufficient to generate a biologically active antibody. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

D. Small Molecules

The present invention concerns HABP2 inhibitors that are small molecules, which refers to a small compound that is biologically active but is not a polymer. It does refer to a monomer. In certain embodiments, the small molecule is inhibits the polyanion binding activity of HABP2 or the serine protease catalytic domain.

### III. DISEASES AND CONDITIONS

The present invention concerns methods and compositions involving higher molecular weight hyaluronan, particularly where low molecular weight hyaluronan has been purified away from the higher molecular weight hyaluronan. Because of the data generated by the inventors, diseases and conditions characterized by or caused by increased vascular permeability are particularly amenable to treatment with such HA compositions. Moreover, these compositions can be used to inhibit angiogenesis to effect a therapeutic benefit in patients suffering from angiogenesis-related diseases and conditions.

Additional embodiments of the invention concern a HABP2 inhibitor for preventing and/or treating diseases and conditions disclosed herein.

A. Vascular Permeability

Vascular permeability refers to the capacity of the wall of a blood vessel to allow small molecules or cells to pass through. Endothelial cells make up blood vessel walls. Diseases or conditions that are characterized by or caused by an increase in vascular permeability include, but are not limited to, acute respiratory distress syndrome (ARDS), acute lung injury (ALI), ventilator-induced lung injury (VILI), sepsis, radiation pneumonitis, tumors, macular degeneration, capillary leakage syndrome, or atherosclerosis.

In particular embodiments of the invention, methods and compositions may be applied to the treatment of ARDS. A number of different therapies have been attempted for this disease with limited success (Table 1).

### Table of Failed/Inconclusive Therapies for ARDS

| Treatment | | |
|---|---|---|
| Modality | Outcome | Reference |
| Early High Dose | No effect on patient mortality in both ARDS and | 1-3 |
| Corticosteroids | Sepsis | |
| Late Low Dose | Methyprednisolone administered through the | 4-5 |
| Corticosteroids | fibroproliferative stage of ARDS reduced | |
| | hospital mortality to 12% from 62% in placebo | |
| | cohort. | |
| Prostaglandin E1 | PGE1 failed to deliver reproducible outcomes in | 6-10 |
| (PGE1) | two trials focused on patients afflicted with | |
| | ARDS emanating from trauma or sepsis. | |
| Inhaled Nitric | NO reduces pulmonary artery pressure but | 11-20 |
| Oxide (NO) | several multicenter clinical trials have shown no | |
| | survival benefit from this treatment. NO also | |
| | causes methemoglobinemia, increased | |
| | pulmonary edema and rebound pulmonary | |
| | hypertension. | |
| Prostacyclin | This vascular smooth muscle relaxant has shown | 21 |
| | similar effects to NO but likewise failed to | |
| | improve survival frequencies in treated | |
| | populations. | |
| Surfactant | Decreased surface tension is predicted to | 22-27 |
| treatment | decrease alveolar collapse so surfactant therapy | |
| | was anticipated to improve ARDS survival but | |
| | clinical trials have not shown improved survival | |
| | metrics. | |
| Lisofylline | This agent inhibits lysophosphatidic | 28 |
| | acyltransferase and decreases cell derived FFA, | |
| | in addition to TNFa, IL-1 and IL-6. Clinical | |
| | trials failed to show efficacy in increasing | |
| | survival | |
| Ketoconazole | This agent inhibits thromboxane and leukotriene | 29-32 |
| | synthesis inhibiting procoagulation activity. | |
| | However, clinical trials failed to show | |
| | statistically significant improvement in end point | |
| | parameters. | |
| Antioxidants | Use of antioxidants, procysteine and N- | 33-36 |
| | acetylcysteine on three human trials was not | |
| | successful in improving oxygenation and | |
| | survival rates in ARDS patients | |
| Immunonutrition | Meta-analysis of 12 randomized controlled | 37-38 |
| | studies comparing enteral nutrition with anti- | |
| | oxidant nutrition revealed no improvement on reducing mortality. | |

B. Angiogenesis

Blood vessels are constructed by two processes: vasculogenesis, whereby a primitive vascular network is established during embryogenesis from multipotential mesenchymal progenitors; and angiogenesis, in which preexisting vessels send out capillary sprouts to produce new vessels. Endothelial cells are centrally involved in each process. They migrate, proliferate and then assemble into tubes with tight cell-cell connections to contain the blood (Hanahan, 1997). Angiogenesis occurs when enzymes, released by endothelial cells, and leukocytes begin to erode the basement membrane, which surrounds the endothelial cells, allowing the endothelial cells to protrude through the membrane. These endothelial cells then begin to migrate in response to angiogenic stimuli, forming offshoots of the blood vessels, and continue to proliferate until the off-shoots merge with each other to form the new vessels.

Normally, angiogenesis occurs in humans and animals in a very limited set of circumstances, such as embryonic development, wound healing, and formation of the corpus luteum, endometrium and placenta.

Examples of diseases associated with neovascularization include tumors, inflammatory conditions, and degenerative conditions. Regarding the eye, non-limiting examples of diseases associated with neovascularization include corneal neovascularization. Corneal neovascularization may be due to contact lens wear, dry eyes, corneal scar formation, pterygia, acne rosasea, cornal surgery such as transplantation or lasik, or inflammatory conditions of the cornea. Another type of neovascularization of the eye is neovascularization of the iris. Neovasculization of the iris may be due to diabetes, neovascular glaucoma, or ocular ischemic syndrome. Causes of retinal neovascularization include proliferative diabetic retinopathy, branch retinal vein occlusion, and central retinal vein occlusion. Another type of neovascularization is neovascularizatio of the optic nerve, which may be caused by conditions such as diabetes mellitus or ocular ischemic syndrome, and choroidal neovascularization.

In particular embodiments, the neovascularization is choroidal neovascularization. Examples of causes of choroidal neovascularization include, but are not limited to, exudative (“wet”) age-related macular degeneration, pathological myopia, angioid streaks, histoplasmosis, sarcoidosis, multifocal choroiditis, punctate inner choroidopathy, nevi, melanoma, retinoblastoma, hemangioma, osteoma, choroidal rupture/trauma, laser photocoagulation, retinopathy of prematurity, and idiopathic.

It is commonly believed that tumor growth is dependent upon angiogenic processes. Thus, the ability to increase or decrease angiogenesis has significant implications for clinical situations, such as wound healing (e.g., graft survival) or cancer therapy, respectively.

Several lines of direct evidence now suggest that angiogenesis is essential for the growth and persistence of solid tumors and their metastases (Folkman, 1989; Kim et al., 1993; Millauer et al., 1994). To stimulate angiogenesis, tumors up-regulate their production of a variety of angiogenic factors, including the fibroblast growth factors (FGF and DTCF) (Kandel et al., 1991) and vascular endothelial cell growth factor/vascular permeability factor (VEGF/VPP). However, many malignant tumors also generate inhibitors of angiogenesis, including angiostatin and thrombospondin (Chen et al., 1995; Good et al., 1990: O'Reilly et al., 1994). It is postulated that the angiogenic phenotype is the result of a net balance between these positive and negative regulators of neovascularization (Good et al., 1990; O'Reilly et al., 1994; Parangi et al., 1996; Rastinejad et al., 1989). Several other endogenous inhibitors of angiogenesis have been identified, although not all are associated with the presence of a tumor. These include, platelet factor 4 (Gupta et al., 1995; Maione et al., 1990), interferon-alpha, interferon-inducible protein 10 (Angiolillo et al., 1995; Strieter et al., 1995), which is induced by interleukin-12 and/or interferon-gamma (Voest et al., 1995), gro-beta (Cao et al., 1995), and the 16 kDa N-terminal fragment of prolactin (Clapp et al., 1993).

Angiogenesis-related diseases may be treated using the methods described in present invention to inhibit endothelial cell proliferation. Angiogenesis-related diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, Rubeosis; Osler-Webber Syndrome; myocardial angiogenesis; plaque neovascularization; telangiectasia; hemophiliac joints; angiofibroma; and wound granulation. The endothelial cell proliferation inhibiting methods of the present invention are useful in the treatment of disease of excessive or abnormal stimulation of endothelial cells. These diseases include, but are not limited to, intestinal adhesions, atherosclerosis. scleroderma, and hypertrophic scars, i.e., keloids. They are also useful in the treatment of diseases that have angiogenesis as a pathologic consequence such as cat scratch disease (Rochele minalia quintosa) and ulcers (Helobacter pylori).

Normal tissue homeostasis is a highly regulated process of cell proliferation and cell death. An imbalance of either cell proliferation or cell death can develop into a cancerous state (Solyanik et al., 1995; Stokke et al., 1997; Mumby and Walter, 1991; Natoli et al., 1998; Magi-Galluzzi et al., 1998). For example, cervical, kidney, lung, pancreatic, colorectal and brain cancer are just a few examples of the many cancers that can result (Erlandsson, 1998; Kolmel, 1998; Mangray and King, 1998; Mougin et al., 1998). In fact, the occurrence of cancer is so high that over 500,000 deaths per year are attributed to cancer in the United States alone.

C. Treatment and Prevention Methods and Compositions

A method of the present invention includes treatment for a disease or condition increased vascular permeability or angiogenesis. An immunogenic polypeptide of the invention can be given to induce an immune response in a person infected with staphylococcus, suspected of having been exposed to staphylococcus, or at risk of exposure to staphylococcus. Methods may be employed with respect to individuals who have tested positive for exposure to staphylococcus or who are deemed to be at risk for infection based on possible exposure.

It is contemplated that compositions of the invention may be administered to a patient within about 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months of being diagnosed with a vascular-permeability-related disease or condition, diagnosed with an angiogenesis-related disease or condition, identified as having symptoms of a vascular-permeability-related or angiogenesis-related disease or condition, or identified as at risk for a vascular-permeability-related or angiogenesis-related disease or condition.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no other treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

In particular embodiments, compositions may be administered 1, 2, 3, 4, 5, 6, 7, 8, 9, 10, 11, 12, 13, 14, 15, 16, 17, 18, 19, 20 or more times, and/or they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range or combination derivable therein.

Compounds and compositions may be administered to a patient intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation, by injection, by infusion, by continuous infusion, by localized perfusion bathing target cells directly, via a catheter, via nebulizer, via aerosol, or via a lavage.

In certain embodiments, the composition is administered intravenously. Examples of other routes of administration, particularly for eye diseases or conditions, include intravitreal administration, intralesional administration, intratumoral administration, topical administration to the surface of the eye, topical application to the surface of a tumor, direct application to a neovascular membrane, subconjunctival administration, periocular administration, retrobulbar administration, subtenon administration, intracameral administration, subretinal administration, posterior juxtascleral administration, and suprachoroidal administration.

D. Combination Therapy

The compositions and related methods of the present invention may also be used in combination with the administration of traditional therapies. Certain embodiments of the present invention also involve including one or more secondary forms of therapy directed to treatment of pathological neovascularization or increased vascular permeability in a subject.

Any secondary therapy known to those of ordinary skill in the art is contemplated by the present invention. For example, the secondary therapy may be pharmacological therapy, surgical therapy, radiation therapy, chemotherapy, laser surgery, cryotherapy, immunotherapy, or gene therapy.

Various combinations may be employed, for example, hyaluronan therapy is “A” and the secondary therapy is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

For ALI or ARDS, typical treatments include methylprednisolone or some other corticosteroid treatment. Other treatments include some form of mechanical ventilation such as airway pressure release ventilation (see World Wide Web at aacn.org/pdfLibra.NSF/Files/ci120205/$file/ci120205.pdf) or low tidal volume (Brower, 2002. Other treatments are shown in Table 1, any of which may be combined with hyaluronan therapy to achieve a greater efficacy. In some embodiments a patient is also given one or more other treatments used for treating the disease or condition. Examples of such treatments include administration of anti-inflammatory drugs, corticosteroids (such as methylprednisolone), NSAIDS, or applying airway pressure release ventilation, or applying other ventilation techniques such as low tidal volume ventilation. A patient may have been treated previously or may be treated concurrently or in the future with such treatments.

In specific embodiments, it is contemplated that a second anti-cancer therapy, such as chemotherapy, radiotherapy, immunotherapy or other gene therapy, is employed in combination with the HA therapy, as described herein.

1. Chemotherapy

The compositions and related methods of the present invention may also be used in combination with the administration of traditional therapies. Certain embodiments of the present invention also involve including one or more secondary forms of therapy directed to treatment of pathological neovascularization in a subject. Any secondary therapy known to those of ordinary skill in the art is contemplated by the present invention. For example, the secondary therapy may be pharmacological therapy, surgical therapy, radiation therapy, chemotherapy, laser surgery, cryotherapy, immunotherapy, or gene therapy.

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

2. Radiotherapy

Other factors that cause DNA damage and have been used extensively include what are commonly known as y-rays, X-rays, and/or the directed delivery of radioisotopes to tumor cells. Other forms of DNA damaging factors are also contemplated such as microwaves, proton beam irradiation (U.S. Pat. No. 5,760,395 and U.S. Pat. No. 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with hyaluronan therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000).

Moreover, antibodies against any of these compounds can be used to target the anti-cancer agents discussed herein.

As discussed earlier, examples of immunotherapies currently under investigation or in use are immune adjuvants e.g., Mycobacterium bovis, Plasmodium falciparum, dinitrochlorobenzene and aromatic compounds (U.S. Pat. No. 5,801,005; U.S. Pat. No. 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. No. 5,830,880 and U.S. Pat. No. 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). It is contemplated that one or more anti-cancer therapies may be employed with the MDA-7 therapies described herein.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie and Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

4. Gene Therapy

In yet another embodiment, a combination treatment involves gene therapy in which a therapeutic polynucleotide is administered before, after, or at the same time as an MDA-7 polypeptide or nucleic acid encoding the polypeptide. Delivery of an MDA-7 polypeptide or encoding nucleic acid in conjunction with a vector encoding one of the following gene products may have a combined therapeutic effect on target tissues. A variety of proteins are encompassed within the invention, some of which are described below.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Macular Degeneration

For example, if the neovascularization is choroidal neovascularization due to age-related macular degeneration in an eye of a subject, then the secondary therapy can be any therapy known to those of ordinary skill in the art that can be applied in the treatment of choroidal neovascularization. The secondary therapy may be pharmacological therapy, laser surgery, surgical therapy other than laser, cryotherapy, vitrectomy, subretinal surgery, or photodynamic therapy involving injection of vertiporfin into the subject. Other examples of secondary therapies include siRNAs, Bevasiranib, anecortave, radiation therapy, retinal or cortical chips, rheopheresis, submacular surgery, and vitamin and mineral supplements (e.g., vitamin E, beta-carotene, zine, copper).

In one aspect, it is contemplated that hyaluronan therapy is used in conjunction with a secondary treatment. Alternatively, the therapy may precede or follow the other agent treatment by intervals ranging from minutes to weeks. In embodiments where the other agents and/or a proteins or polynucleotides are administered separately, one would generally ensure that a significant period of time did not expire between each delivery, such that the agent and antigenic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

E. General Pharmaceutical Compositions

In some embodiments, pharmaceutical compositions are administered to a subject. Different aspects of the present invention involve administering an effective amount of a composition to a subject. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-cancer agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including inhalants and the like.

The active compounds of the present invention can be formulated for parenteral administration, e.g., formulated for injection via the intravenous, intramuscular, sub-cutaneous, or even intraperitoneal routes. The preparation of an aqueous composition that contains a compound or compounds that increase the expression of an MHC class I molecule will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. A solution may be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9% or more hyaluronan, or any range derivable therein.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions according to the present invention will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

### II. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Hyaluronan Regulation of Endothelial Cell Barrier Function

A. Materials and Methods

Abbreviations: ATP—Adenosine 5′-triphosphate, HA—Hyaluronan, HGF—Hepatocyte Growth Factor, PDGF—Platelet-Derived Growth Factor, S1P—Sphingosine 1-phosphate, VEGF—Vascular Endothelial Growth Factor.

Cell Culture and Reagents—Human pulmonary artery EC were obtained from Cambrex (Walkersville, Md.) and cultured as previously described in EBM-2 complete medium (Cambrex) at 37° C. in a humidified atmosphere of 5% CO₂, 95% air, with passages 6-10 used for experimentation (Garcia et al., 2001). Unless otherwise specified, reagents were obtained from Sigma (St. Louis, Mo.). Reagents for SDS-PAGE electrophoresis were purchased from Bio-Rad (Richmond, Calif.), Immobilon-P transfer membrane from Millipore (Millipore Corp., Bedford, Mass.), and gold microelectrodes from Applied Biophysics (Troy, N.Y.). Rat anti-CD44 (IM-7, common domain) antibody was purchased from BD Biosciences (San Diego, Calif.). Goat anti-CD44var (v3-v10) antibody and mouse anti-KDR (VEGF receptor 2) antibody were purchased from Chemicon, International (Temecula, Calif.). Rabbit anti-CD44v3, anti-CD44v6 and anti-CD44v10 antibody were purchased from Calbiochem (San Diego, Calif.). Rabbit anti-caveolin-1, anti-flotillin-1, anti-lamin A/C, anti-GRP75, anti-GRP 78, anti-GRASP65, anti-vimentin, anti-AKT1, anti-phospho-threonine(308) AKT, anti-phospho-serine(473) AKT, anti-ROCK1, anti-ROCK2, anti-p115 rhoGEF and anti-Tiam1 antibodies were purchased from Santa Cruz Biotechnology (Santa Cruz, Calif.). Rabbit anti-S1P₁ receptor was purchased from Affinity Bioreagents (Golden, Colo.). Rabbit anti-phospho-serine and anti-phospho-threonine antibodies were purchased from Zymed Laboratories, Inc. (South San Francisco, Calif.). Mouse antibodies were purchased for S1P₃ receptor (Exalpha Biologicals, Watertown, Mass.), RhoA, Rac1, pp60src and phospho-tyrosine antibody (Upstate Biotechnology, Lake Placid, N.Y.). Mouse anti-β-actin antibody and rabbit anti-phospho-tyrosine(418) Src antibody were purchased from Sigma (St. Louis, Mo.). Recombinant active Src, ROCK1 and ROCK2 were purchased from Upstate Biotechnology (Lake Placid, N.Y.). Secondary horseradish peroxidase (HRP)-labeled antibodies were purchased from Amersham Biosciences (Piscataway, N.J.). Texas Red-conjugated phalloidin was purchased from Molecular Probes (Eugene, Oreg.).

Preparation and Quantitation of Low and High MW Hyaluronan (HA)—The method of preparation is similar to that described (Slevin et al., 2002). For HMW-HA, rooster comb HA (500 mg, ˜1 million Da polymers) (Bourguignon et al., 2004) was dissolved in distilled water and centrifuged in an Ultrafree-MC™ Millipore 100,000 Da MW cutoff filter (Bedford, Mass.) after which the flow through (less than 100,000 Da) was discarded. For LMW-HA, 500 mg of rooster comb HA was digested with 20,000 U of bovine testicular hyaluronidase in digestion buffer (0.1 M sodium acetate, pH=5.4, 0.15 M NaCl) for 24 hours. The reaction was stopped with 10% trichloroacetic acid. The resulting solution was centrifuged in an Ultrafree-MC™ Millipore 5,000 Da MW cutoff filter (Bedford, Mass.) after which the flow through (less than 5,000 Da) was dialyzed against distilled water for 24 hours at 4° C. in 500 Da cutoff Spectra-Por tubing (Pierce-Warriner, Chester, UK). Low and High MW HA were quantitated using an ELISA-like competitive binding assay with a known amount of fixed HA and biotinylated HA binding peptide (HABP) as the indicator (Pogrel et al., 2003). In some cases, both Low and High MW HA were subject to boiling, proteinase K (50 μg/ml) digestion, hyaluronidase SD digestion (100 mU/ml) or addition of boiled (inactivated) hyluronidase SD to test for possible protein/lipid contaminants (Calabro et al., 2000). LMW and HMW-HA with DNA standards were run on 4-20% SDS-PAGE gels and stained with combined Alcian blue and silver staining to further determine HA purity and size (Min and Cowman, 1986).

Lipid Raft Isolation—Caveolin-enriched microdomain known as lipid rafts were isolated from human lung EC as described (Singleton and Bourguignon, 2004; Singleton et al., 2005). Materials insoluble in Triton X-100-insoluble were mixed with 0.6 ml of cold 60% Optiprep™ and then overlaid with 0.6 ml of 40%-20% Optiprep™. The resulting gradients were then centrifuged (35,000 rpm) in SW60 rotor for 12 h at 4° C. and different fractions were collected and analyzed. In some cases, different fractions were analyzed for total cholesterol content using a cholesterol assay kit (Amplex Red™, Invitrogen (Molecular Probes), Eugene, Oreg.).

Immunoprecipitation and Immunoblotting—Cellular materials associated within the 20% Optiprep™ fractions (lipid raft fraction) were incubated with IP buffer A (50 mM HEPES (pH 7.5), 150 mM NaCl, 20 mM MgCl₂, 1% Nonidet P-40 (NP-40), 0.4 mM Na₃VO₄, 40 mM NaF, 50 μM okadaic acid, 0.2 mM phenylmethylsulfonyl fluoride, 1:250 dilution of Calbiochem protease inhibitor mixture 3) or IP buffer B (50 mM HEPES (pH 7.5), 150 mM NaCl, 20 mM MgCl₂, 1% Triton X-100, 0.1% SDS, 0.4 mM Na₃VO₄, 40 mM NaF, 50 μM okadaic acid, 0.2 mM phenylmethylsulfonyl fluoride, 1:250 dilution of Calbiochem protease inhibitor mixture 3) as indicated. The samples were then immunoprecipitated with anti-S1P₁ receptor or anti-S1P₃ receptor IgG followed by SDS-PAGE in 4-15% polyacrylamide gels, and transfer onto Immobilon™ membranes. Development occurred using specific primary and secondary antibodies. Enhanced chemiluminescence (Amersham Biosciences) for visualization of immunoreactive bands then took place.

Total RNA Isolation—To isolate total RNA, Trizol LS (Invitrogen, Carlsbad, Calif.) was employed, followed by RNeasy column (Qiagen Inc., Valencia, Calif.) for further purification.

Reverse-transcriptase Polymerase Chain Reaction (RT-PCR)— Transcript levels of selected CD44 isoforms were measured using SuperScript One-Step RT-PCR with Platinum Taq system (Invitrogen Inc., Carlsbad, Calif.) according to the manufacturer's protocol. Specific primer pairs were used as follows: for all CD44 isoforms (C2A reverse primer: 5′-CCAAGATGATCAGCCATTCTGG-3′ (SEQ ID NO:3), GenBank #L05422, and C13 Forward Primer: 5′AAGACATCTACCCCAGCAAC-3′ (SEQ ID NO:4), GenBank #L05410) and for CD44v10-specific isoform (C2A reverse primer: 5′-CCAAGATGATCAGCCATTCTGG-3′ (SEQ ID NO:3), GenBank #L05422, and pv10 Forward Primer: GGTGGAAGAAGAGACCCAAA-3′ (SEQ ID NO:5), GenBank #L05419). Amplicons were analyzed by 1.25% agarose gel electrophoresis in 1×TBE.

Determination of Complex Formation between S1P₁ Receptor/CD44s and S1P₃ Receptor/CD44v10—EC monolayers were serum-starved for one hour, then treated with High or Low MW HA (100 nM) (5-30 min.) and solubilized in IP buffer A (see above). Following immunoprecipitation with either rabbit anti-S1P₁ receptor or mouse anti-S1P₃ receptor antibody, the samples were subjected to SDS-PAGE in 4-15% polyacrylamide gels and transfer onto Immobilon™ membranes (Millipore Corp., Bedford, Mass.). Nonspecific sites were then blocked with 5% bovine serum albumin, and the blots were then incubated with either rat anti-CD44 (IM-7, common domain) antibody, rabbit anti-S1P₁ antibody or mouse anti-S1P₃ antibody followed by incubation with horseradish peroxidase (HRP)-labeled goat anti-rabbit, goat anti-mouse or goat anti-rat IgG. Enhanced chemiluminescence (Amersham Biosciences) for visualization of immunoreactive bands then took place.

Determination of Tyrosine/Serine/Threonine Phosphorylation of the S1P₁ and S1P₃ Receptor—Immunoprecipitation of solubilized proteins in IP buffer B (see above) took place with either rabbit anti-S1P₁ receptor or mouse anti-S1P₃ receptor antibody, which was followed by SDS-PAGE in 4-15% polyacrylamide gels and transfer onto Immobilon™ membranes (Millipore Corp., Bedford, Mass.). Following blockage of nonspecific sites with 5% bovine serum albumin, the blots were incubated with either rabbit anti-S1P₁ antibody, mouse anti-S1P₃ antibody, mouse anti-phospho-tyrosine, rabbit anti-phospho-serine antibody or rabbit anti-phospho-threonine antibody, and then incubated with horseradish peroxidase (HRP)-labeled goat anti-rabbit or goat anti-mouse IgG. Enhanced chemiluminescence (Amersham Biosciences) for visualization of immunoreactive bands then took place.

Construction and Transfection of siRNA against S1P₁, S1P₃, CD44, AKT1, Src, ROCK1, ROCK2, Rac1 and RhoA—The siRNA sequence(s) targeting human against S1P₁, S1P₃, CD44, Rac1 and RhoA were generated using mRNA sequences from Gen-Bank™ (gi:13027635, gi:38788192, gi:30353932, gi:62241010, gi:77415509, gi:4885582, gi:41872582, gi:29792301, gi:33876092 respectively). For each mRNA (or scramble), two targets were identified: S1P₁ target sequence 1 (5′-AAGCTACACAAAAAGCCTGGA-3′ (SEQ ID NO:6)), S1P₁ target sequence 2 (5′-AAAAAGCCTGGATCACTCATC-3′ (SEQ ID NO:7)), S1P₃ target sequence 1 (5′-AACAGGGACTCAGGGACCAGA-3′ (SEQ ID NO:8)), S1P₃ target sequence 2 (5′-AAATGAATGTTCCTGGGGCGC-3′ (SEQ ID NO:9)), CD44 target sequence 1 (5′-AATATAACCTGCCGCTTTGCA-3′ (SEQ ID NO:10)), CD44 target sequence 2 (5′-AAAAATGGTCGCTACAGCATC-3′ (SEQ ID NO:11)), AKT1 target sequence 1 (5′-AATTATGGGTCTGTAACCACC-3′ (SEQ ID NO:12)), AKT1 target sequence 2 (5′-AAATGAATGAACCAGATTCAG-3′ (SEQ ID NO:13)), Src target sequence 1 (5′-AAAATCGAACCTCAGTGGCGG-3′ (SEQ ID NO:14)), Src target sequence 2 (5′-AATCGAACCTCAGTGGCGGCG-3′ (SEQ ID NO:15)), ROCK1 target sequence 1 (5′-AAAAAATGGACAACCTGCTGC-3′ (SEQ ID NO:16)), ROCK1 target sequence 2 (5′-AAGTGAATTCGGATTGTTTGC-3′ (SEQ ID NO:17)), ROCK2 target sequence 1 (5′-AATCTGACTGAGGGGCGGGGA-3′ (SEQ ID NO:18)), ROCK2 target sequence 2 (5′-AAGCCGGAGCTAGAGGCAGGC-3′ (SEQ ID NO:19)), Rac1 target sequence 1 (5′-AAAACTTGCCTACTGATCAGT-3′ (SEQ ID NO:20)), Rac1 target sequence 2 (5′-AACTTGCCTACTGATCAGTTA-3′ (SEQ ID NO:21)), RhoA target sequence 1 (5′-AAGAAACTGGTGATTGTTGGT-3′ (SEQ ID NO:22)), RhoA target sequence 2 (5′-AAAGACATGCTTGCTCATAGT-3′ (SEQ ID NO:23)), scrambled sequence 1 (5′-AAGAGAAATCGAAACCGAAAA-3′ (SEQ ID NO:24)) and scramble sequence 2 (5′-AAGAACCCAATTAAGCGCAAG-3′ (SEQ ID NO:25)). The Johns Hopkins University DNA Analysis Facility provided sense and antisense oligonucleotides. A transcription-based kit was used (Silencer™ siRNA construction kit (Ambion, Tex.)) to generate the siRNA. Human lung EC were then transfected with siRNA using siPORTamine™ as the transfection reagent (Ambion, Tex.) according to the manufacturer's protocol. Cells (˜40% confluent) were serum-starved for 1 hour followed by incubation with 3 μM (1.5 μM of each siRNA) of target siRNA, or scramble siRNA or no siRNA, for 6 hours in serum-free media. The serum-containing media was then added (1% serum final concentration) for 42 h before biochemical experiments and/or functional assays were conducted.

Rho Family Activation Assay—The RhoA and Rac activation assays using human lung EC were performed as described (Ren et al., 1999).

Measurement of TransEC Electrical Resistance (TER)—EC were grown to confluence in polycarbonate wells containing evaporated gold microelectrodes. TER measurements were performed using an electrical cell-substrate impedance sensing system (Applied Biophysics, Troy, N.Y.) as described (Garcia et al., 2001). TER values from each microelectrode were pooled at discrete time points and plotted versus time as the mean±S.E.

In Vitro S1P Receptor Phosphorylation—The S1P receptor phosphorylation reaction took place using 50 μl of a reaction mixture containing 40 mM Tris-HCl (pH 7.5), 2 mM EDTA, 1 mM dithiothreitol, 7 mM MgCl₂, 0.1% CHAPS, 0.1 μM calyculin A, 100 μM ATP, purified enzymes (i.e. 100 ng of recombinant active Src, ROCK1 or ROCK2) with or without immunoprecipitated S1P₁ or S1P₃ receptor obtained from human pulmonary EC that were serum-starved for one hour. After incubation for 30 min at 30° C., the reaction mixtures were boiled in SDS sample buffer and subjected to SDS-PAGE. Mouse anti-phospho-tyrosine, rabbit anti-phospho-serine, rabbit anti-phospho-threonine, rabbit anti-S1P₁ or mouse anti-S1P₃ antibody were used to perform immunoblots, followed by incubation with horseradish peroxidase (HRP)-labeled goat anti-rabbit or goat anti-mouse IgG. Enhanced chemiluminescence (Amersham Biosciences) for visualization of immunoreactive bands then took place.

Immunofluorescence Microscopy and Cortical Actin Quantitation—Polymerized actin rearrangement was assessed first with Texas Red-conjugated phalloidin (Invitrogen (Molecular Probes), Eugene, Oreg.) and then analyzed using a Nikon Eclipse TE 300 microscope as described (Garcia et al., 2001). ImageQuant™ software (Amersham Biosciences Corp. (Piscataway, N.J.)) was used to analyze the computer recorded .tiff images. A standardized average grey value (SAGV) was generated for total phalloidin staining versus cortical phalloidin staining for each cell (Singleton et al., 2005). The following equation was used to calculate percent cortical actin staining: ((cortical actin SAGV×area) divided by (total actin SAGV×area))×100. At least ten cells per sample were analyzed. Experiments were performed in triplicate.

Statistical Analysis—Student's t test was used to compare the means of data from two or more different experimental groups. Results are expressed as means±S.E.\

B. Results

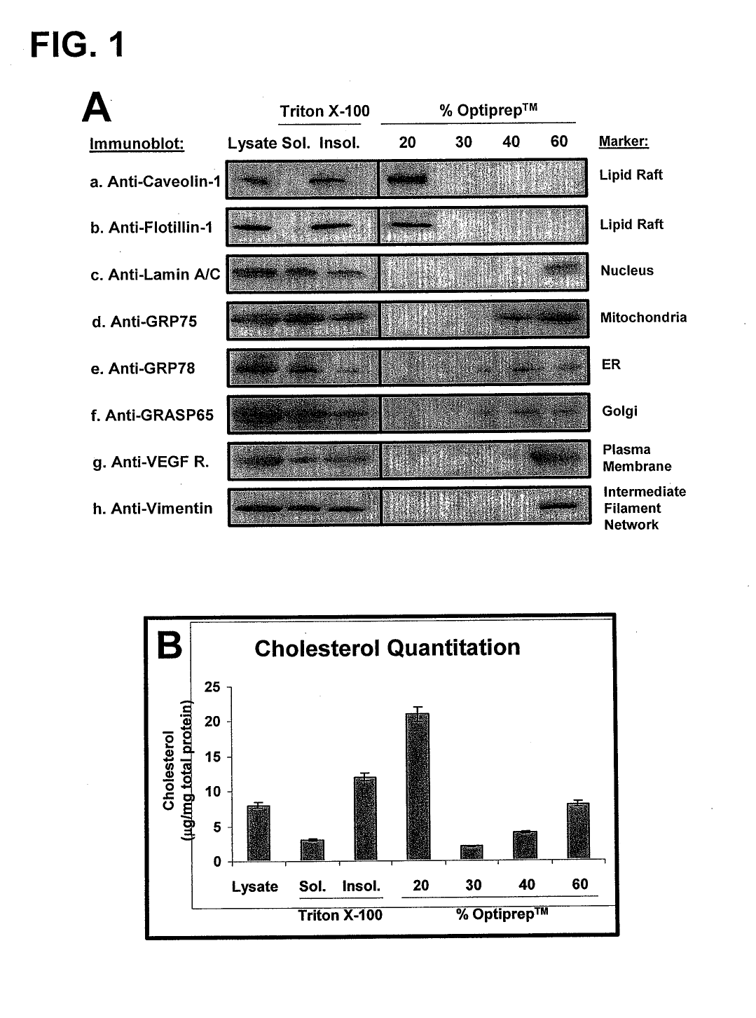

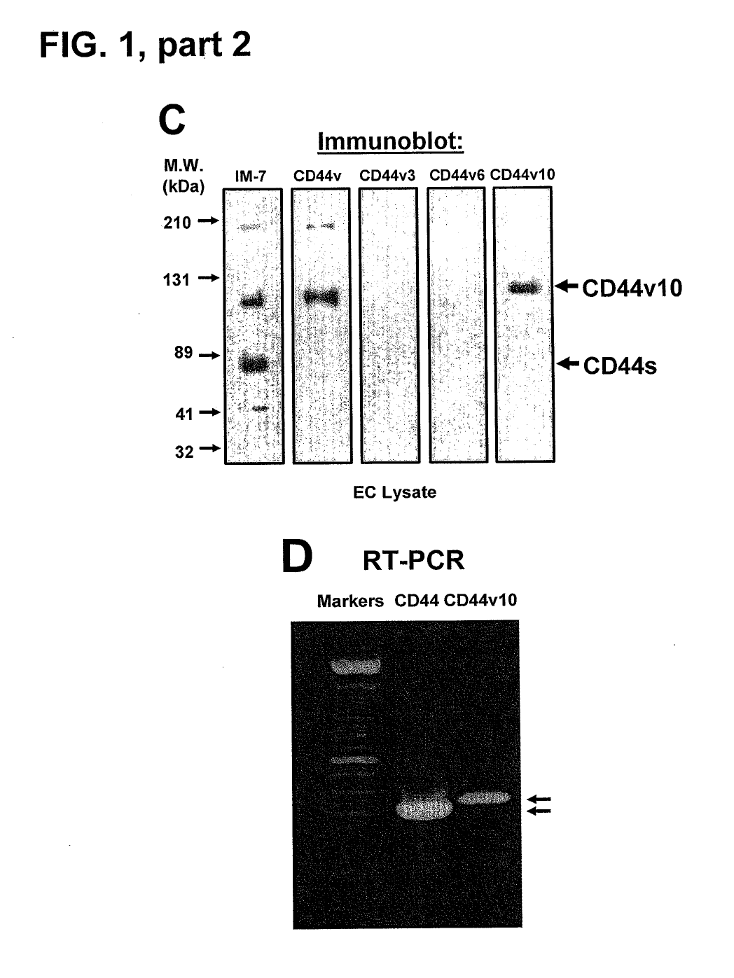

Divergent effects of low and high molecular weight hyaluronan on human lung endothelial cell barrier function. Role of caveolin-enriched microdomains (lipid rafts). The effects of low and high MW-HA on human lung EC barrier function and the role of CD44 and lipid rafts in this process were examined. Lipid rafts, isolated from human lung EC, contain specific markers (caveolin-1 and flotillin-1) and are enriched in cholesterol and do not contain other subcellular organelle markers, as shown in FIG. 1-A and -B. These results demonstrate the purity and specificity of the lipid raft isolation procedure. CD44 isoforms make up a major cell surface HA receptor family: RT-PCR and isoform-specific immunoblot analyses were then performed to explore whether these isoforms were present in human lung EC. FIG. 1-C and -D demonstrate that human pulmonary EC express at least two major CD44 isoforms: CD44s (standard form, MW ˜85 kDa) and CD44v10 (MW ˜116 kDa).

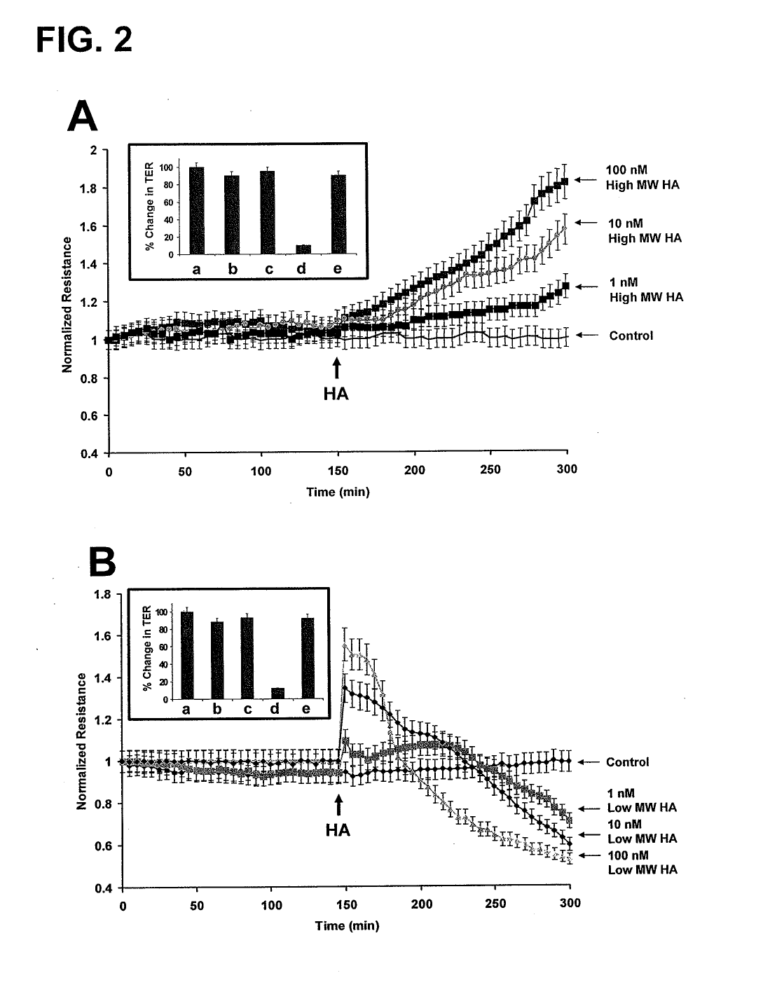

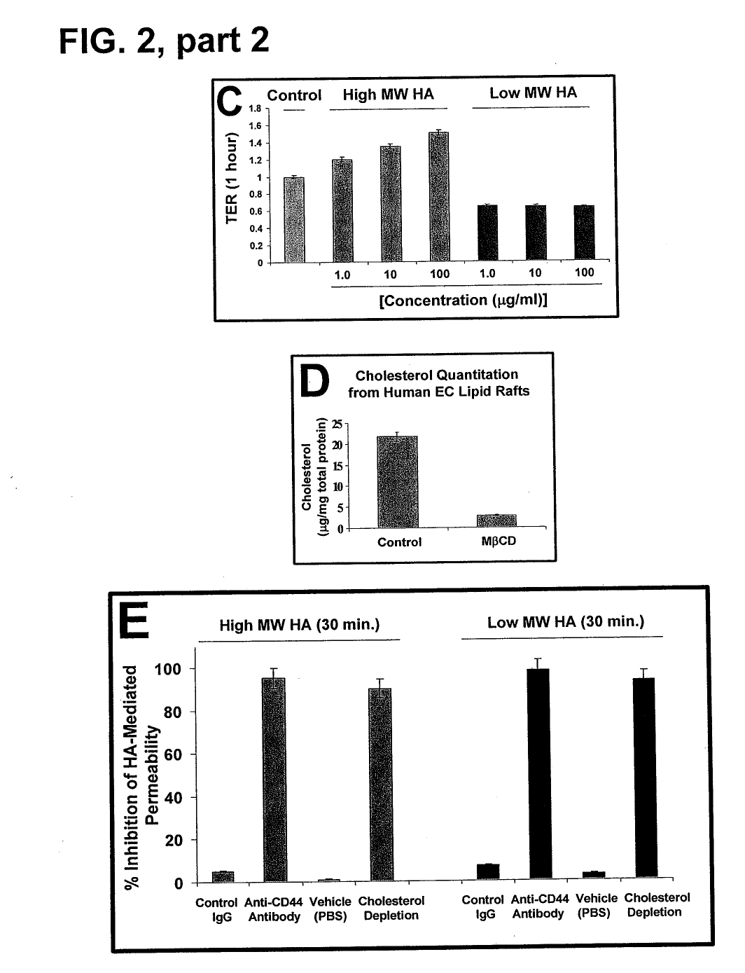

HMW-HA (˜1 million Da) consistently produced a gradual and sustained rise in transmonolayer electrical resistance (TER) in a dose-dependent fashion. LMW-HA (˜2,500 Da) induced biphasic changes in TER with an initial rapid increase in barrier enhancement followed by significant and prolonged barrier disruption (FIG. 2-A and -B). The dose-response was significant in certain situations (e.g., comparing equal nM concentrations), but not in others (equal concentrations in the range of 1.0 to 100 μg/ml (FIG. 2-C)) of Low and High MW HA). Depleting cholesterol with methyl-β-cyclodextrin (MβCD) treatment (FIG. 2-D) or using a CD44 blocking antibody that blocks HA binding to all CD44 isoforms abolished both HMW- and LMW-HA-induced changes in TER (FIG. 2-E). These results demonstrate that HA-mediated EC barrier function is regulated by cholesterol-enriched microdomains. Moreover, CD44 is the major HA receptor responsible for HA-mediated EC barrier alterations.

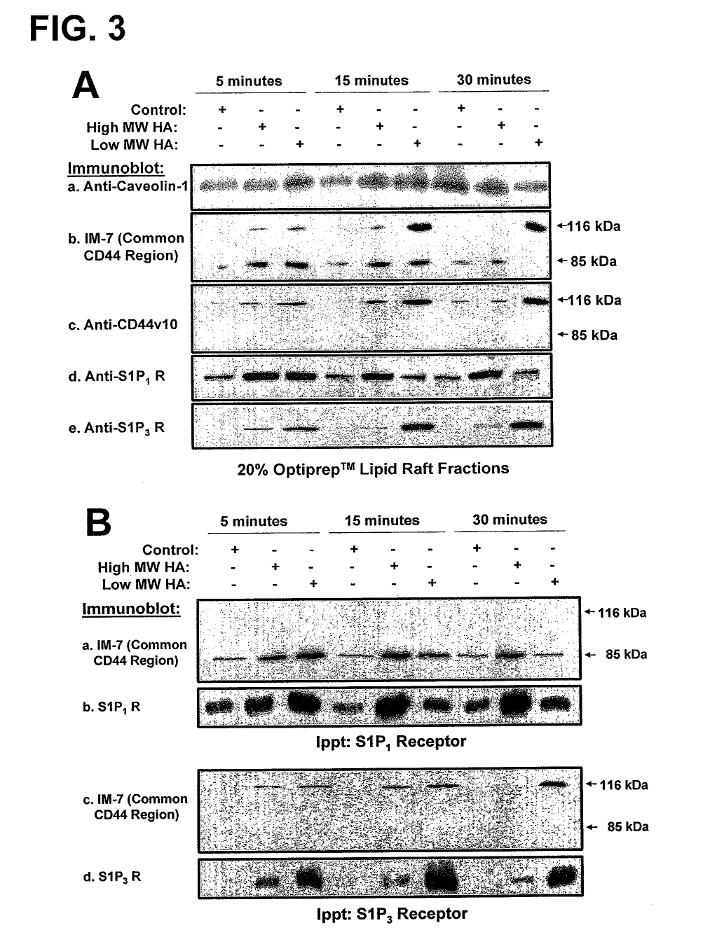

CD44 localizes in activated EC to specialized cholesterol- and caveolin-enriched lipid rafts, plasma membrane microdomains that are implicated in a variety of cellular functions including potocytosis, cholesterol and calcium regulation as well as signal transduction (Singleton and Bourguignon, 2004; Singleton and Bourguignon, 2002; Minshall et al., 2003). Lipid rafts are biochemically defined by insolubility in 4° C. Triton X-100 and light buoyant density after discontinuous gradient centrifugation (Harder and Simons, 1997). Both HMW-HA and LMW-HA rapidly (5 min.) recruit CD44s to the lipid raft fraction while LMW-HA promotes robust but delayed recruitment of CD44v10 (after 15 min.) (FIG. 3-A).

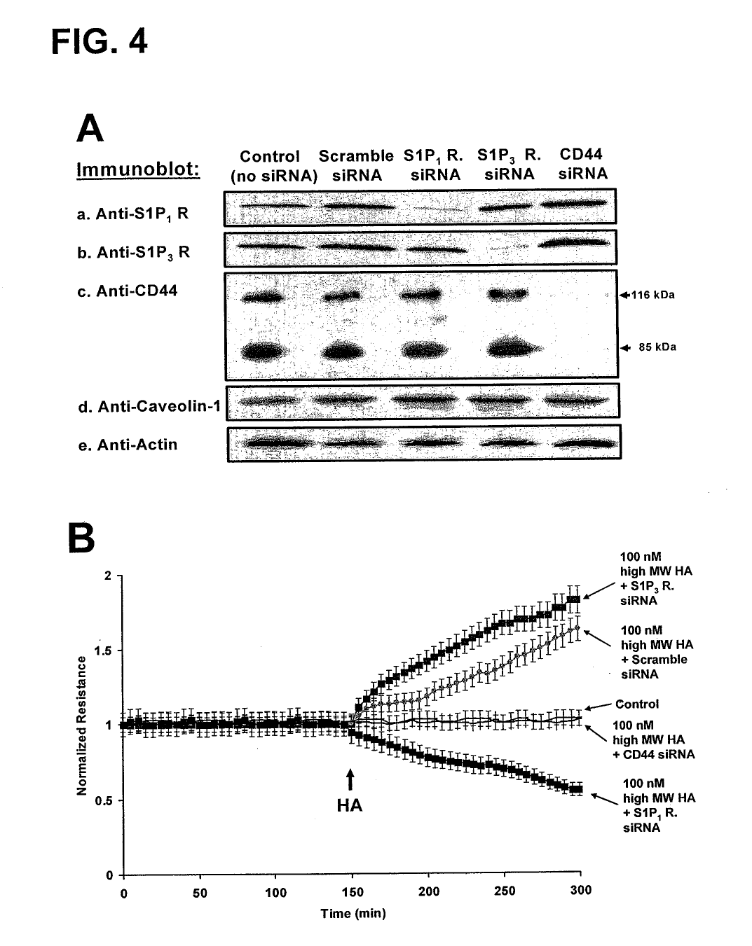

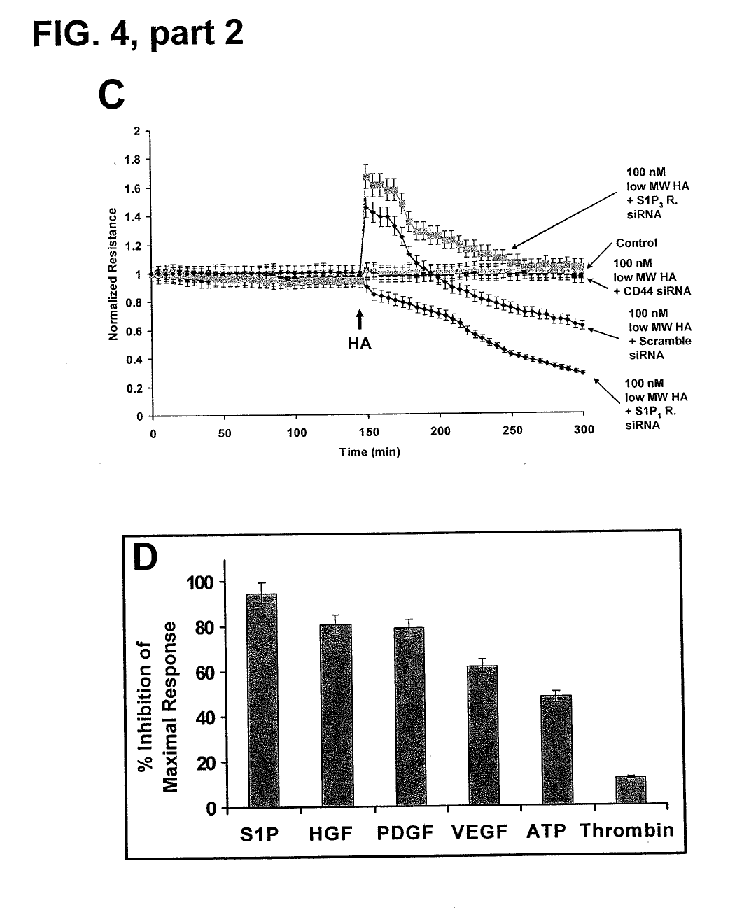

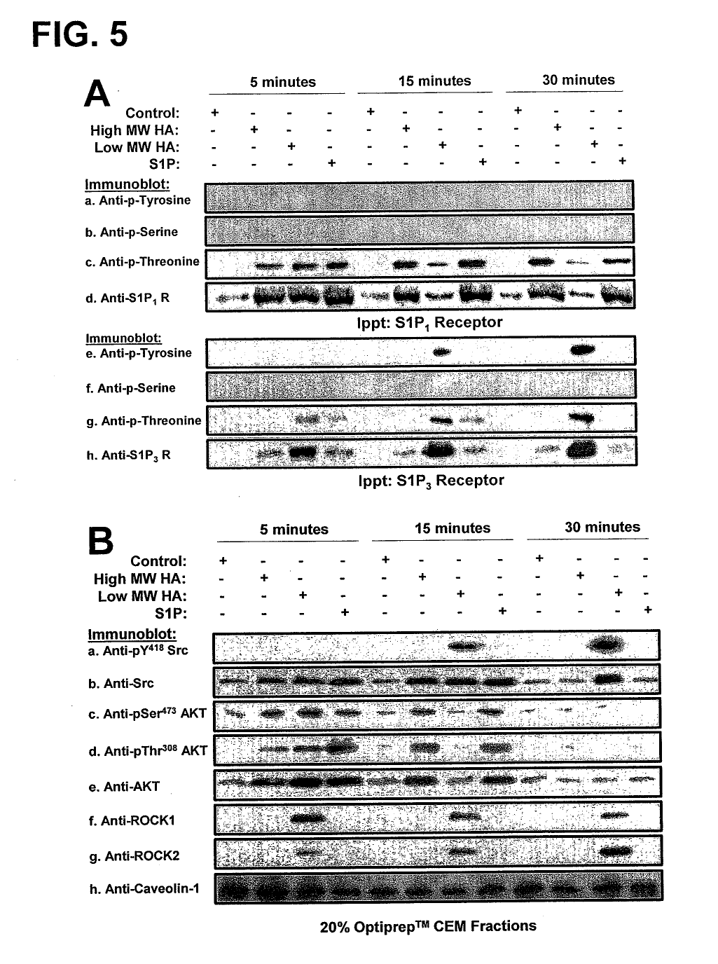

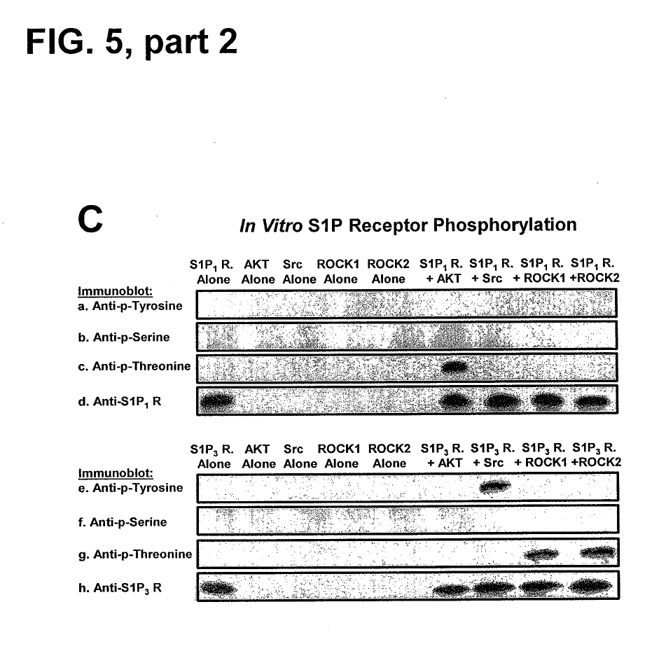

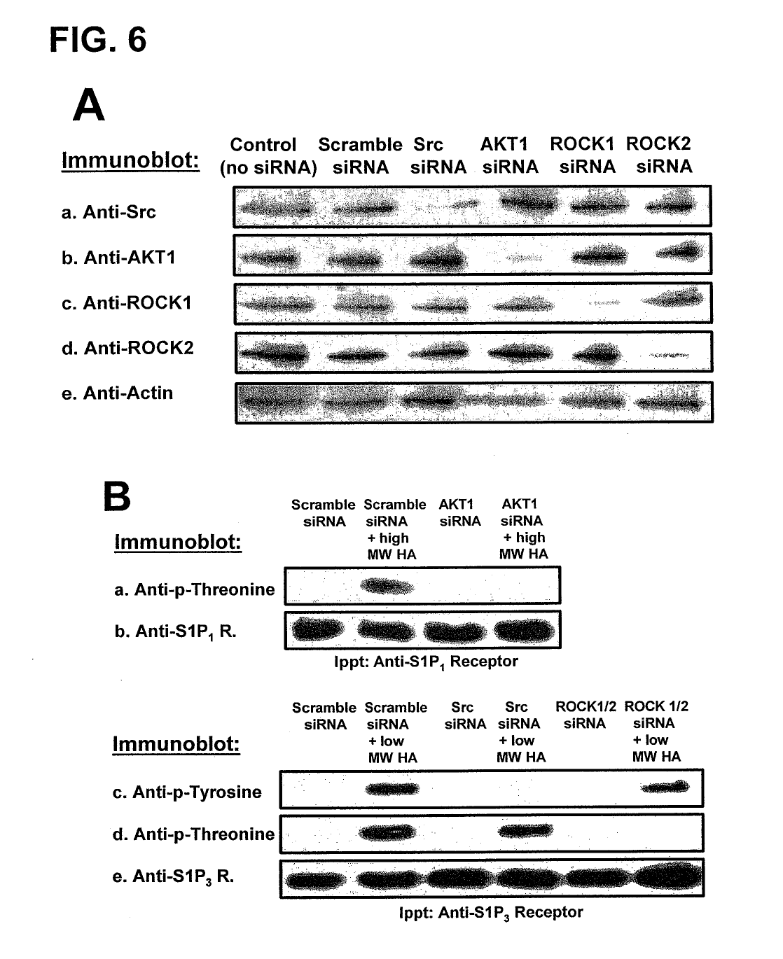

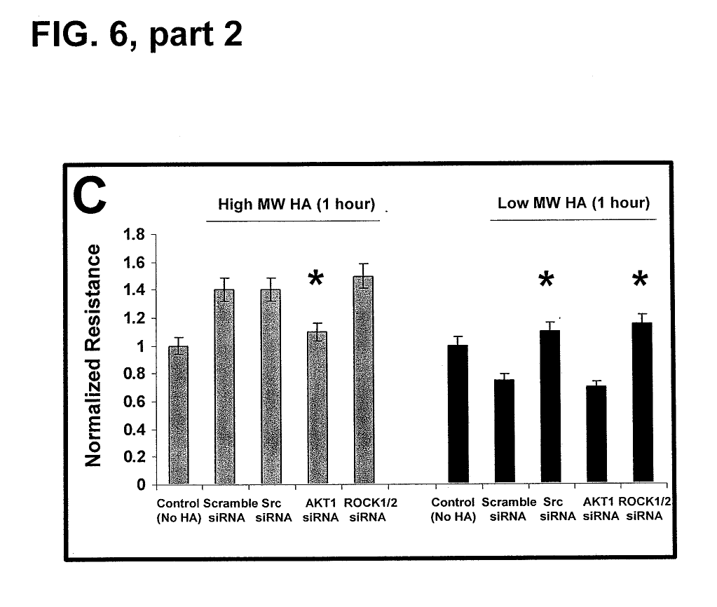

Transactivation of sphingosine 1-phosphate (S1P) receptors are involved in HA-mediated lung vascular barrier regulation in a CD44 isoform-specific manner. Whether HA induces physical and/or functional associations between CD44 and S1P receptors was explored. These entities may be involved in HA-mediated vascular barrier responses. As shown in FIG. 3-B, HMW-HA (100 nM) induced CD44s association in lipid rafts with S1P₁, the known barrier-promoting S1P receptor. In contrast, LMW-HA initially recruited the S1P₁ receptor followed by recruitment of S1P₃ receptors. Immunoprecipitation followed by immunoblotting from lipid raft fractions revealed that HMW-HA promotes S1P₁ receptor association with CD44s. LMW-HA (100 nM), however, induced an initial CD44s association with S1P₁ which was followed by CD44v10 association with S1P₃ receptor in lipid raft fractions. Three different actions eliminated both spatially-specific actin cytoskeletal reorganization and TER alterations evoked by HMW-HA and LMW-HA: MCD (to inhibit lipid raft formation); anti-CD44 blocking antibody; and siRNAs specific for CD44 (FIGS. 2, 4, Table 2). Silencing S1P₁ receptor blocked the EC barrier enhancing effects of High MW HA (FIG. 4). Silencing S1P₃ receptor blocked the EC barrier disruptive effects of Low MW HA (FIG. 4). Consistent with HA-mediated S1P transactivation, HMW-HA promoted AKT1-mediated threonine phosphorylation of S1P₁ receptor while LMW-HA induced sequential AKT1-mediated S1P₁ and Src/ROCK1/2-mediated S1P₃ receptor phosphorylation/activation (FIGS. 5, 6). As shown by FIG. 5-C, these results were confirmed by using in vitro phosphorylation of SP receptors with recombinant AKT1, Src, ROCK1 and ROCK2. Further, silencing AKT1 expression blocks HWM-HA-mediated EC barrier enhancement while silencing Src or both ROCK 1 and 2 expression blocks LMW-HA-mediated EC barrier disruption (FIG. 6-C). Thus, low and high MW HA promote differential CD44 isoform-specific association with and activation of S1P receptors in lipid rafts. Activation of S1P₁ receptor is required for HA-induced EC barrier enhancement while S1P₃ receptor activation promotes barrier disruption.

TABLE 2
Cortical Actin Quantitation

| Cortical Actin Phalloidin Staining/ | 5 Minute | 30 Minute |
|---|---|---|
| Total Phalloidin Staining | Treatment | Treatment |
| 1. Control (Scramble siRNA) | 8 +/− 0.4 | 8 +/− 0.4 |
| 2. High MW HA + Scramble siRNA | 73 +/− 3.5 | 85 +/− 4.2 |
| 3. High MW HA + S1P1 Receptor siRNA | 15 +/− 0.8 | 18 +/− 1.2 |
| 4. High MW HA + S1P3 Receptor siRNA | 75 +/− 3.8 | 88 +/− 4.5 |
| 5. High MW HA + Src siRNA | 74 +/− 3.6 | 84 +/− 4.3 |
| 6. High MW HA + AKT1 siRNA | 22 +/− 1.2 | 25 +/− 1.0 |
| 7. High MW HA + ROCK 1/2 siRNA | 71 +/− 3.2 | 83 +/− 3.9 |
| 8. High MW HA + RhoA siRNA | 78 +/− 3.7 | 84 +/− 3.8 |
| 9. High MW HA + Rac1 siRNA | 22 +/− 0.9 | 25 +/− 1.3 |
| 10. Low MW HA + Scramble siRNA | 64 +/− 2.8 | 5 +/− 0.2 |
| 11. Low MW HA + S1P1 Receptor siRNA | 14 +/− 0.7 | 3 +/− 0.1 |
| 12. Low MW HA + S1P3 Receptor siRNA | 68 +/− 2.7 | 56 +/− 2.5 |
| 13. Low MW HA + Src siRNA | 21 +/− 1.3 | 15 +/− 0.6 |
| 14. Low MW HA + AKT1 siRNA | 65 +/− 2.4 | 52 +/− 2.1 |
| 15. Low MW HA + ROCK 1/2 siRNA | 12 +/− 0.5 | 10 +/− 0.4 |
| 16. Low MW HA + RhoA siRNA | 59 +/− 2.5 | 53 +/− 2.3 |
| 17. Low MW HA + Rac1 siRNA | 18 +/− 0.8 | 4 +/− 0.1 |

Polymerized actin rearrangement was assessed first with Texas Red-conjugated phalloidin (Invitrogen (Molecular Probes), Eugene, OR) and then analyzed using a Nikon Eclipse TE 300 microscope as described (Garcia et al., 2001). ImageQuant ™ software (Amersham Biosciences Corp. (Piscataway, NJ)) was used to analyze the computer recorded .tiff images. A standardized average grey value (SAGV) was generated for total phalloidin staining versus cortical phalloidin staining for each cell (Singleton et al., 2005). The following equation was used to calculate percent cortical actin staining: ((cortical actin SAGV × area) divided by (total actin SAGV × area)) × 100. At least ten cells per sample were analyzed. Experiments were performed in triplicate. Results are expressed as means ± Standard Deviation.

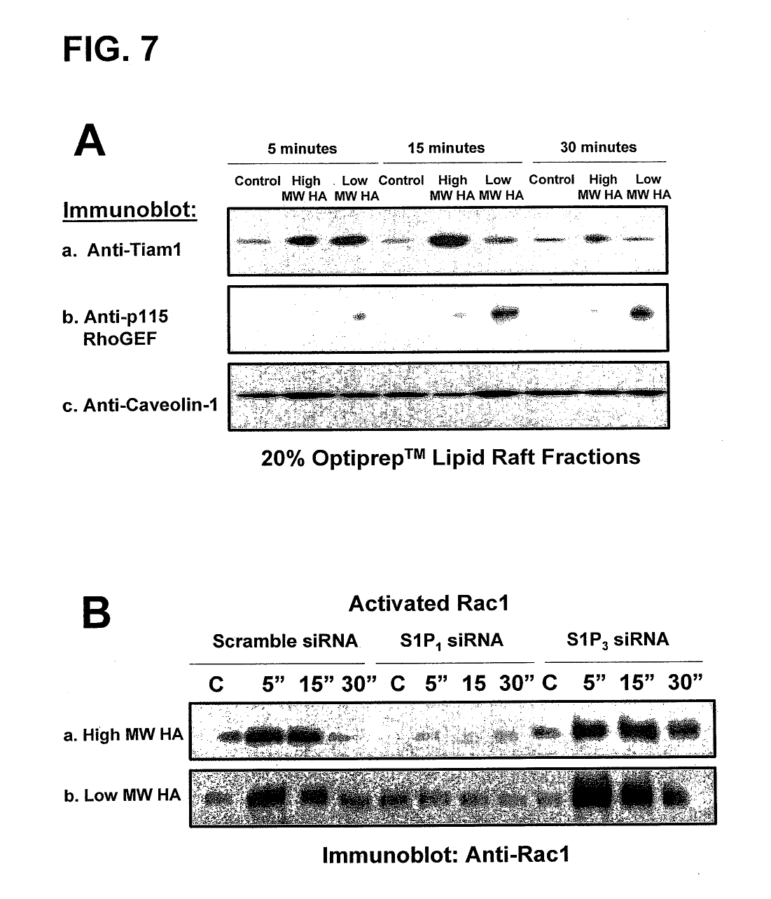

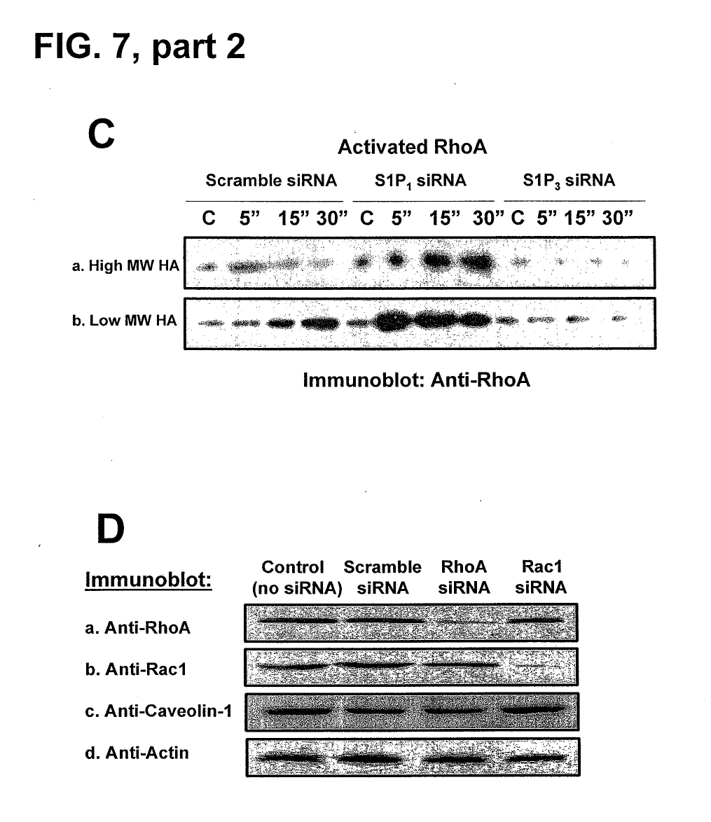

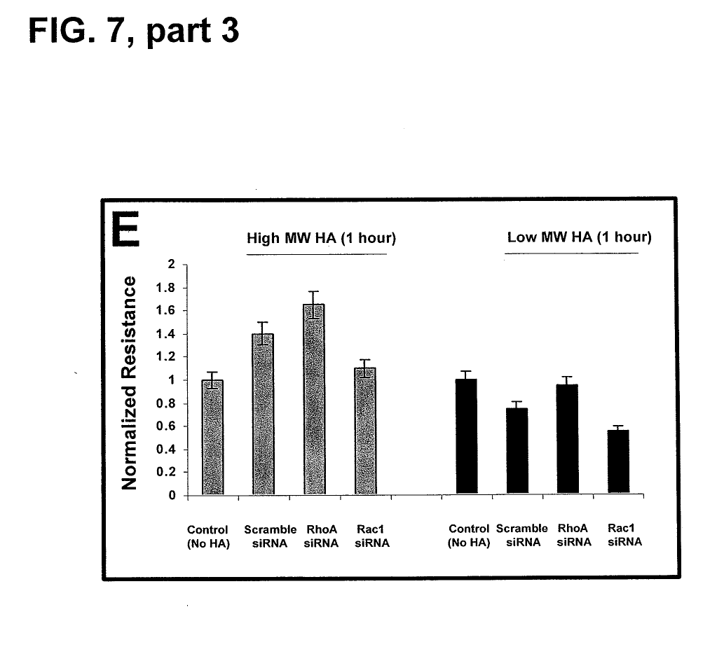

Role of RhoA and Rac1 signaling on HA-induced EC barrier function. The Rho family GTPase, Rac1, regulates S1P-mediated EC barrier enhancement (Garcia et al., 2001). Whether Rho family GTPases could play a role in the HA-specific regulatory responses was examined. The present inventors identified that either LMW-HA (5 min.) or HMW-HA (5, 15, 30 min.) induced Rac1 activation with concomitant recruitment of the Rac1-specific exchange factor, Tiam1, to EC lipid rafts (FIG. 7). Rac1 activation was inhibited by siRNA for S1P₁ (but not S1P₃) to reduce receptor expression. HA-induced EC barrier enhancement was inhibited by silencing Rac1 (but not RhoA) expression. In contrast, LMW-HA (as opposed to HMW-HA) recruited the RhoA exchange factor, p115 RhoGEF, to EC lipid rafts at 15-30 min. and promoted RhoA activation. LMW-HA-induced RhoA activation was inhibited by siRNA for S1P₃ (but not S1P₁) and LMW-HA-induced EC barrier disruption was inhibited by silencing RhoA (but not Rac1) expression.

Silencing either S1P₁ or Rac1 expression attenuated EC barrier-enhancing effects of HMW-HA and LMW-HA. Silencing S1P₃ or RhoA expression diminished the EC barrier-disruptive response to LMW-HA (FIGS. 4, 7). The inventors believe that HA promotes cytoskeletal reorganization and EC barrier regulation through differential CD44 isoform interaction with S1P receptors via RhoA/Rac1 signaling in lipid rafts. Moreover, transactivation of S1P₁ receptor may represent a common mechanism for receptor-mediated vascular barrier regulation.

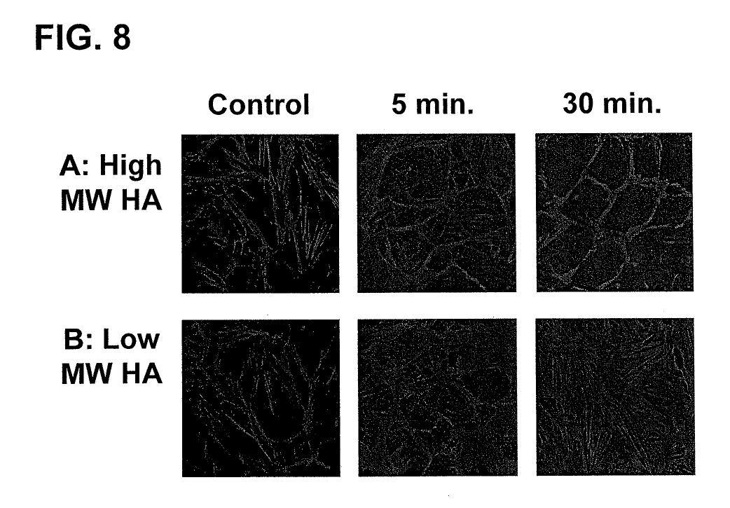

HA-induced, CD44 and S1P receptor-dependent, cytoskeletal reorganization in EC. Transendothelial electrical resistance (TER) measurements of EC barrier function in vitro showed that reduction in expression of either S1P₁ or Rac1 attenuated the barrier-enhancing effects of low and high MW HA, whereas reduction in S1P₃ or RhoA expression attenuated the delayed barrier-disruptive response to low MW HA on EC (FIGS. 4, 7). Since cytoskeletal reorganization is a fundamental element of virtually all EC barrier-regulatory responses, phalloidin staining of HA- and S1P-challenged EC was compared to visualize cellular F-actin localization (FIG. 8, Table 2). Both low and high MW HA induced prominent cortical actin ring formation early on (e.g., timepoint 5 min.). This formation was attenuated by reduction of S1P₁ (but not S1P₃), AKT or Rac1 (but not RhoA) expression. These findings are similar to that reported for S1P (Garcia et al., 2001; Singleton et al., 2005). Low MW HA challenge for 30 min., however, resulted in a loss of cortical actin staining with increased F-actin stress fiber formation which was significantly attenuated by silencing S1P₃ (but not S1P₁) or RhoA (but not Rac1) expression.

Role of S1P₁ receptor as a central regulator of EC permeability. The S1P₁ receptor regulates activated protein C (APC)/endothelial cell protein C receptor (EPCR)-mediated EC barrier protection against edemagenic agents (e.g., thrombin) (Finigan et al., 2005). Since silencing the S1P₁ receptor has been observed to reduce the barrier enhancement induced by HA, and both LMW-HA and HMW-HA promote transactivation of S1P₁ receptor during the EC barrier-enhancing stages of these agonists, whether S1P₁ receptor serves as a central regulator of EC barrier function (FIG. 4-D) was examined. Reductions in S1P₁ receptor expression significantly modulated the barrier-regulatory effects of human lung EC challenged with HGF, PDGF, VEGF or ATP (Garcia et al., 2001; Dudek et al., 2004). In contrast, thrombin, a known EC barrier-disruptive agent, was unaffected by S1P₁ receptor silencing. This result suggests that the S1P₁ receptor serves as a critical and central regulator of EC barrier function.

In summary, HA promotes cytoskeletal reorganization and EC barrier regulation via differential CD44 isoform interaction with SP receptors and RhoA/Rac1 signaling in lipid rafts. In particular, high MW HA induces cortical actin ring formation while low MW HA treatment of EC for short periods of time (e.g., less than 30 min.) promotes actin stress fiber formation. These results demonstrate a requirement for S1P₁ receptor transactivation in agonist-induced EC barrier enhancement. S1P₁ activation may represent a potential common mechanism for receptor-mediated vascular barrier regulation.

### Example 2

### Involvement OF CD44 with Hepatocyte Growth Factor-Mediated Vascular Integrity

A. Materials and Methods

Abbreviations: EC—endothelial cell, HGF—hepatocyte growth factor, CEM—caveolin-enriched microdomain, DRM—detergent-resistant membrane, HMW-HA—high molecular weight hyaluronan, HPMVEC—human pulmonary microvascular endothelial cell, S1P—sphingosine 1-phosphate, Tiam1—T-lymphoma invasion and metastasis gene 1, LPS—lipopolysaccharide, TGFβ—transforming growth factor beta, TNFα—tumor necrosis factor alpha, Gab1—Grb-2-associated binder-1, ERM—ezrin, radixin, moesin, ROCK—rho kinase, PKC—protein kinase C, DAG—diacylglycerol, PIP₂—Phosphatidylinositol-4,5-bisphosphate, NHE1—sodium-hydrogen exchanger 1, MARCKS—myristoylated alanine-rich protein kinase C substrate.

Cell Culture and Reagents—Human pulmonary microvascular EC (HPMVEC) were obtained from Cambrex (Walkersville, Md.) and cultured in EBM-2 complete medium (Cambrex) at 37° C. in a humidified atmosphere of 5% CO₂, 95% air, with passages 6-10 used for experimentation. See ref. (Garcia et al., 2001). Unless otherwise specified, reagents were obtained from Sigma (St. Louis, Mo.). Reagents for SDS-PAGE electrophoresis were purchased from Bio-Rad (Richmond, Calif.), Immobilon-P transfer membrane from Millipore (Millipore Corp., Bedford, Mass.), and gold microelectrodes from Applied Biophysics (Troy, N.Y.). Recombinant human hepatocyte growth factor (HGF), rabbit anti-vWF (Factor VIII) antibody, goat anti-CD44var (v3-v10) antibody and mouse anti-KDR (VEGF receptor 2) antibody were purchased from Chemicon International (Temecula, Calif.). Rat anti-CD44 (IM-7, common domain) antibody was purchased from BD Biosciences (San Diego, Calif.). Rabbit anti-phospho-c-Met (Tyr1234/1235), rabbit anti-phospho-c-Met (Tyr1349) and mouse anti-c-Met antibodies were purchased from Cell Signaling Technology (Boston, Mass.). Rabbit anti-CD44v3, anti-CD44v6 and anti-CD44v10 antibody were purchased from Calbiochem (San Diego, Calif.). FITC-conjugated anti-CD44 (HCAM) antibody was purchased from Abcam (Cambridge, Mass.). Rabbit anti-phospho-serine antibody was purchased from Zymed Laboratories, Inc. (South San Francisco, Calif.). Rabbit anti-dynamin 2, rabbit anti-Tiam1 and rabbit anti-caveolin-1 antibodies were purchased from Santa Cruz Biotechnology (Santa Cruz, Calif.). Mouse anti-Rac1 and mouse anti-cortactin antibodies were obtained from Upstate Biotechnology (Lake Placid, N.Y.). Mouse anti-β-actin antibody, lipopolysaccharide (LPS) and Optiprep™ were purchased from Sigma (St. Louis, Mo.). Secondary horseradish peroxidase (HRP)-labeled antibodies were purchased from Amersham Biosciences (Piscataway, N.J.).

Caveolin-enriched microdomain (CEM) isolation—Caveolin-enriched microdomain known as detergent-resistant membranes (DRM) or lipid rafts were isolated from HPMVEC (Singleton et al., 2006; Singleton et al., 2005). Triton X-100-insoluble materials were mixed with 0.6 ml of cold 60% Optiprep™ and then overlaid with 0.6 ml of 40%-20% Optiprep™. Gradients were centrifuged (35,000 rpm) in SW60 rotor for 12 h at 4° C. and different fractions were collected and analyzed.

Immunoprecipitation and Immunoblotting—Cellular materials from treated or untreated HPMVEC were incubated with IP buffer (50 mM HEPES (pH 7.5), 150 mM NaCl, 20 mM MgCl₂, 1% Nonidet P-40 (NP-40), 0.4 mM Na₃VO₄, 40 mM NaF, 50 μM okadaic acid, 0.2 mM phenylmethylsulfonyl fluoride, 1:250 dilution of Calbiochem protease inhibitor mixture 3). Following immunoprecipitation with either anti-CD44 or anti-dynamin 2 IgG, the samples were subjected to SDS-PAGE in 4-15% polyacrylamide gels, transferred onto Immobilon™ membranes, and developed with specific primary and secondary antibodies. Enhanced chemiluminescence (Amersham Biosciences) was used to visualize immunoreactive bands. In some instances, standardized average grey values (S.A.G.V., processed from ImageQuant™ software (Amersham Biosciences)) were obtained for immunoreactive bands for quantification.

Construction and Transfection of siRNA Against c-Met, CD44, Tiam1, Cortactin, Dynamin 2, Rac1 and the S1P₁ Receptor—The siRNA sequence(s) targeting human c-Met, CD44, Tiam1, cortactin, dynamin 2, Rac1 and the S1P₁ receptor were generated using mRNA sequences from Gen-Bank™ (gi:427-41654, gi: 30353932, gi:897556, gi:20357555, gi:32451864, gi: 29792301, gi:13027635 respectively). For each mRNA (or scramble), two targets were identified. Specifically, c-Met target sequence 1 (5′-AAAGATAAACCTCTCATAATG-3′ (SEQ ID NO:26)), c-Met target sequence 2 (5′-AAACCTCTCATAATGAAGGCC-3′ (SEQ ID NO:27)), CD44 target sequence 1 (5′-AATATAACCTGCCGCTTTGCA-3′ (SEQ ID NO:10)), CD44 target sequence 2 (5′-AAAAATGGTCGCTACAGCATC-3′ (SEQ ID NO:11)), Tiam1 target sequence 1 (5′-AAACAGCTTCAGAAGCCTGAC-3′ (SEQ ID NO:28)), Tiam1 target sequence 2 (5′-AATGCTCTGAATCCTAGTCTC-3′ (SEQ ID NO:29)), cortactin target sequence 1 (5′-AATGCCTGGAAATTCCTCATT-3′ (SEQ ID NO:30)), cortactin target sequence 2 (5′-AAACAGAATTTCGTGAACAGC-3′ (SEQ ID NO:31)), dynamin 2 target sequence 1 (5′-AACATGCCGAGTTTTTGCACT-3′ (SEQ ID NO:32)), dynamin 2 target sequence 2 (5′-AAACAGAACATGCCGAGTTTT-3′ (SEQ ID NO:33)), Rac1 target sequence 1 (5′-AAAACTTGCCTACTGATCAGT-3′ (SEQ ID NO:34)), Rac1 target sequence 2 (5′-AACTTGCCTACTGATCAGTTA-3′ (SEQ ID NO:21)), S1P₁ target sequence 1 (5′-AAGCTACACAAAAAGCCTGGA-3′ (SEQ ID NO:6)), S1P₁ target sequence 2 (5′-AAAAAGCCTGGATCACTCATC-3′ (SEQ ID NO:7)), scrambled sequence 1 (5′-AAGAGAAATCGAAACCGAAAA-3′ (SEQ ID NO:24)) and scramble sequence 2 (5′-AAGAACCCAATTAAGCGCAAG-3′ (SEQ ID NO:25)) were utilized. Sense and antisense oligonucleotides were purchased from Integrated DNA Technologies (Coralville, Iowa). A transcription-based kit was used to generate the siRNA (Silencer™ siRNA construction kit (Ambion, Tex.)). Human lung EC were then transfected with siRNA using siPORTamine™ as the transfection reagent (Ambion, Tex.) according to the manufacturer's protocol. Cells (approximately 40% confluent) were serum-starved for 1 h followed by incubation with 31.1M (1.5 μM of each siRNA) of target siRNA, scramble siRNA or no siRNA for 6 hours in serum-free media. The serum-containing media was then added (10% serum final concentration) for 42 h before biochemical experiments and/or functional assays were conducted.

Preparation and Quantitation of High MW Hyaluronan (HA)—This method follows a previously reported procedure (Singleton et al., 2006; Slevin et al., 2002). Briefly, 500 mg of rooster comb HA (−1 million Da polymers) (Bourguignon et al., 2004) was dissolved in distilled water and centrifuged in an Ultrafree-MC™ Millipore 100,000 Da MW cutoff filter (Bedford, Mass.) and the flow through (less than 100,000 Da) was discarded. High MW HA was quantitated using an ELISA-like competitive binding assay with a known amount of fixed HA and biotinylated HA binding peptide (HABP) as the indicator (Pogrel et al., 2003). HMW-HA with DNA standards were run on 4-20% SDS-PAGE gels and stained with combined Alcian blue and silver staining to further determine HA purity and size (Min and Cowman, 1986).

Determination of serine phosphorylation of CD44—Solubilized CEM proteins in IP buffer (see above) were immunoprecipitated with rat anti-CD44 antibody followed by SDS-PAGE in 4-15% polyacrylamide gels and transfer onto Immobilon™ membranes (Millipore Corp., Bedford, Mass.). After blocking nonspecific sites with 5% bovine serum albumin, blots were incubated with either rat anti-CD44 antibody or rabbit anti-phospho-serine antibody followed by incubation with horseradish peroxidase (HRP)-labeled goat anti-rabbit or goat anti-rat IgG. Enhanced chemiluminescence (Amersham Biosciences) was used to visualize immunoreactive bands.

Determination of Complex Formation between CD44 and c-Met—Solubilized CEM proteins in IP buffer (see above) were immunoprecipitated with rat anti-CD44 antibody or anti-c-Met antibody followed by SDS-PAGE in 4-15% polyacrylamide gels and transfer onto Immobilon™ membranes (Millipore Corp., Bedford, Mass.). Nonspecific sites were blocked with 5% bovine serum albumin, and then the blots were incubated with either rat anti-CD44 antibody or mouse anti-c-Met antibody followed by incubation with horseradish peroxidase (HRP)-labeled goat anti-mouse or goat anti-rat IgG. Visualization of immunoreactive bands was achieved using enhanced chemiluminescence (Amersham Biosciences).

Measurement of EC Electrical Resistance—EC were grown to confluence in polycarbonate wells containing evaporated gold microelectrodes, and TER measurements were performed using an electrical cell-substrate impedance sensing system obtained from Applied Biophysics (Troy, N.Y.) as described (Garcia et al., 2001). TER values from each microelectrode were pooled at discrete time points and plotted versus time as the mean±S.E.

Rac1 Activation Assay—Rac1 activity assays were performed as described (Ren et al., 1999) in human lung EC.

Determination of Complex Formation between Dynamin 2 and Cortactin/Caveolin-1—Solubilized CEM proteins in IP buffer (see above) were immunoprecipitated with rabbit anti-dynamin 2 antibody followed by SDS-PAGE in 4-15% polyacrylamide gels and transfer onto Immobilon™ membranes (Millipore Corp., Bedford, Mass.). Nonspecific sites were blocked with 5% bovine serum albumin, and then the blots were incubated with either rabbit anti-dynamin 2 antibody, mouse anti-cortactin antibody, rabbit anti-caveolin-1 antibody or rabbit anti-Tiam1 antibody followed by incubation with horseradish peroxidase (HRP)-labeled goat anti-rabbit or goat anti-mouse IgG. Visualization of immunoreactive bands was achieved using enhanced chemiluminescence (Amersham Biosciences).

Animal Preparation and Treatment—Male C57BL/6J and CD44 knockout mice (8-10 weeks, Jackson Laboratories, Bar Harbor, Me.) were anesthetized with intraperitoneal ketamine (150 mg/kg) and acetylpromazine (15 mg/kg) according to approved protocols. LPS (2.5 mg/kg) or saline (control) were instilled intratracheally and four hours later, HGF (50 μg/kg) or saline control delivered intravenously through the internal jugular vein. The animals were allowed to recover for 24 h followed by bronchioalveolar lavage protein analysis and/or lung immunohistochemistry.

Murine Lung Immunohistochemistry—The following protocol was used to characterize protein expression in mouse lung vascular endothelial cells (EC). Lungs from C57BL/6J control (untreated) mice were formalin fixed, and 5 micron paraffin sections were obtained, hydrated and subjected to epitope retrieval (DakoCytomation Target Retrieval Solution, pH=6.0, DakoCytomation, Carpinteria, Calif.). The sections were then histologically evaluated by either FITC-conjugated anti-CD44 antibody or anti-c-Met or anti-Factor VIII (vWF) antibody and secondary secondary fluorescent antibody (Alexa Fluor™ 610 (for vWF) and 350 (for c-Met), Molecular Probes (Invitrogen, Carlsbad, Calif.)). Negative controls for immunohistochemical analysis were performed by the same method as above but without primary antibody. Immunofluorescent stained sections were photographed (100×) using a Leica Axioscope (Bannockburn, Ill.).

Determination of Bronchioalveolar Lavage Protein Concentration—Bronchioalveolar lavage (BAL) was performed by an intratracheal injection of 1 cc of Hank's balanced salt solution followed by gentle aspiration. The recovered fluid was processed for protein concentration (BCA Protein Assay Kit; Pierce Chemical Co., Rockford, Ill.) as described (Su et al., 2004).

Statistical Analysis—Student's t test was used to compare the means of data from two or more different experimental groups. Results are expressed as means±S.E.

B. Results

Role of CD44 in HGF/c-Met-mediated human EC barrier enhancement. The mechanism by which hepatocyte growth factor (HGF) binds to its plasma membrane receptor tyrosine kinase, c-Met, and induces cellular function (Hammond et al., 2004; Kermorgant and Parker, 2005; Ma et al., 2003) (including endothelial cell (EC) barrier displacement (Liu et al., 2002)), is not well defined. A major hyaluronan (HA) receptor localized in caveolin-enriched microdomains (CEM), CD44, may play a role in regulating HGF/c-Met signaling (van der Voort et al., 1999; Taher et al., 1999; Orian-Rousseau et al., 2002; Orian-Rousseau et al., 2007). Participation of CD44 in HGF-induced EC barrier regulation was thus studied by the present inventors.

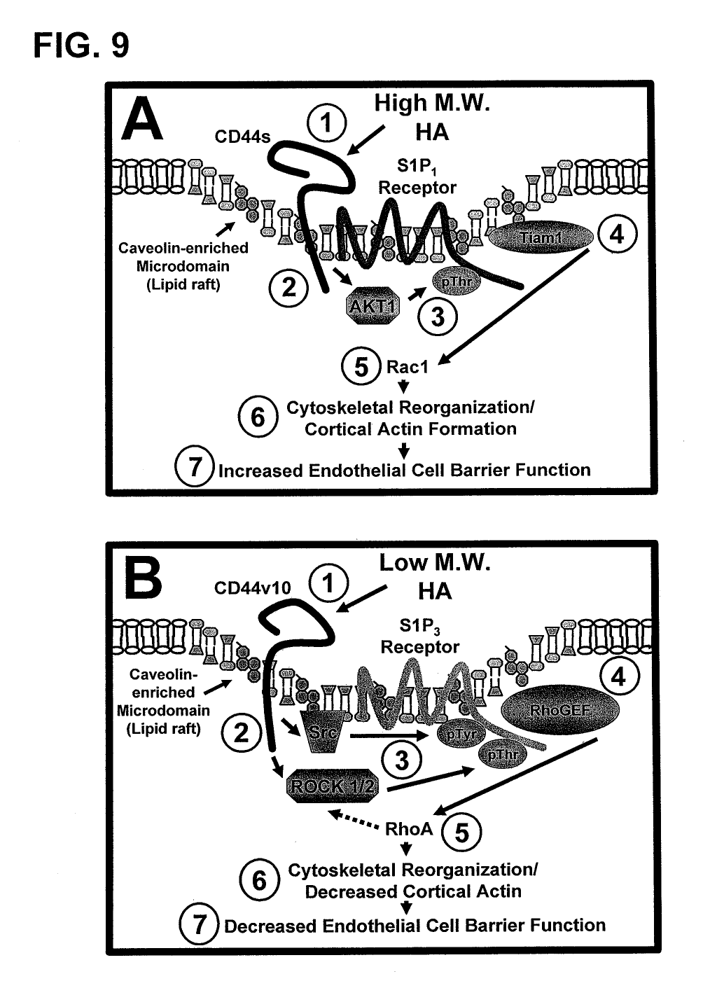

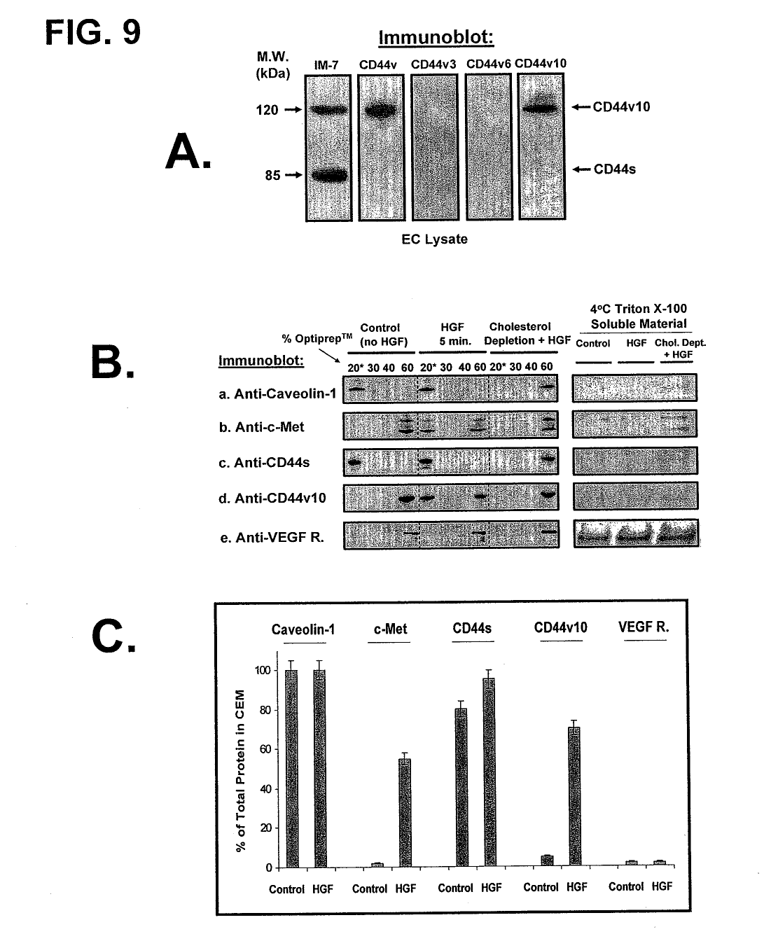

The data described herein indicate that there are two main CD44 isoforms expressed in human pulmonary EC: CD44v10, with a weight of ˜120 kDa, and CD44s (standard form, ˜85 kDa) (FIG. 9-A). In the absence of HGF (control), CD44s, but not CD44v10 or c-Met, localizes to caveolin-enriched microdomains (CEM, also termed detergent-resistant membranes or lipid rafts). HGF (25 ng/ml) treatment of human EC induced recruitment of ˜70% of total CD44v10 and ˜55% of total c-Met into CD44s-containing CEM (FIG. 9-B and -C).

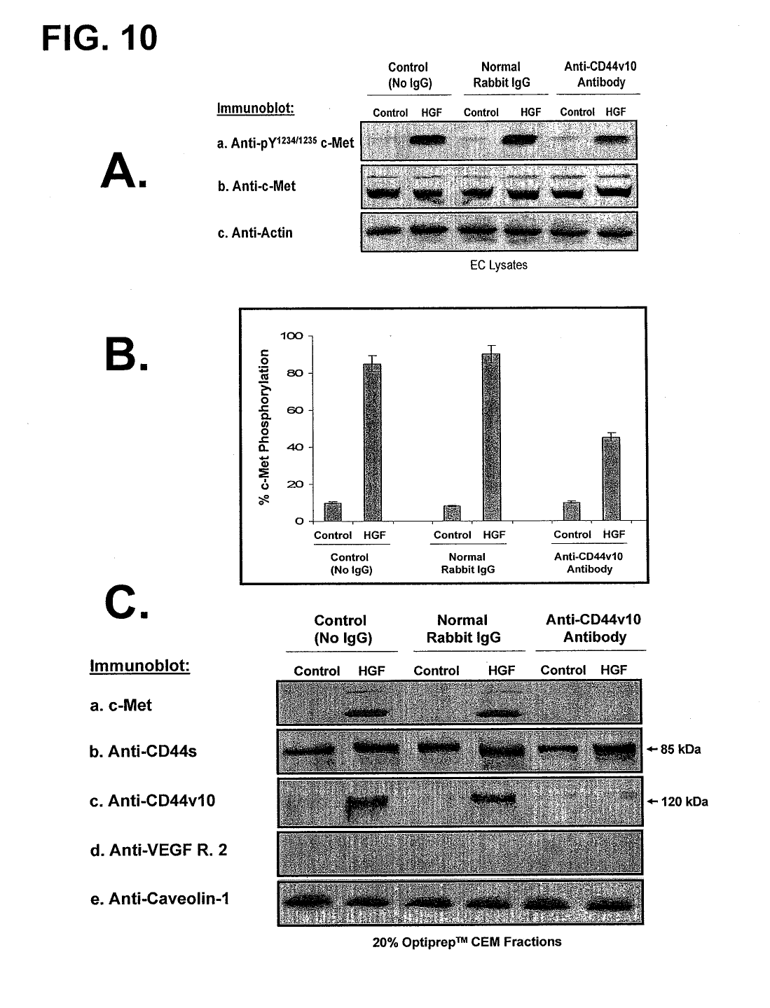

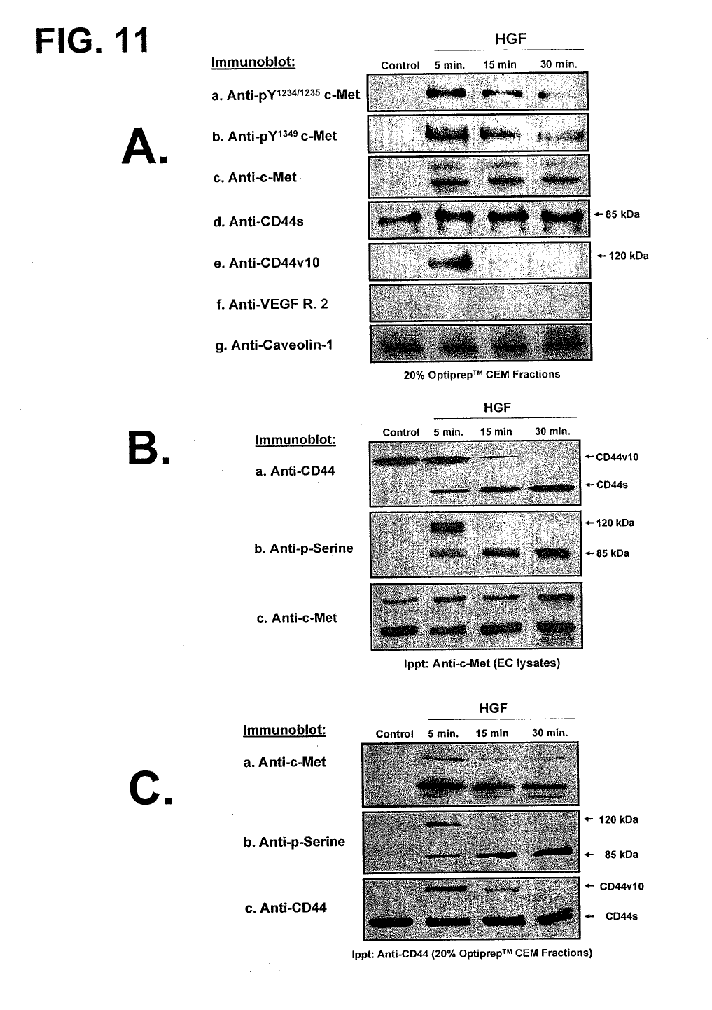

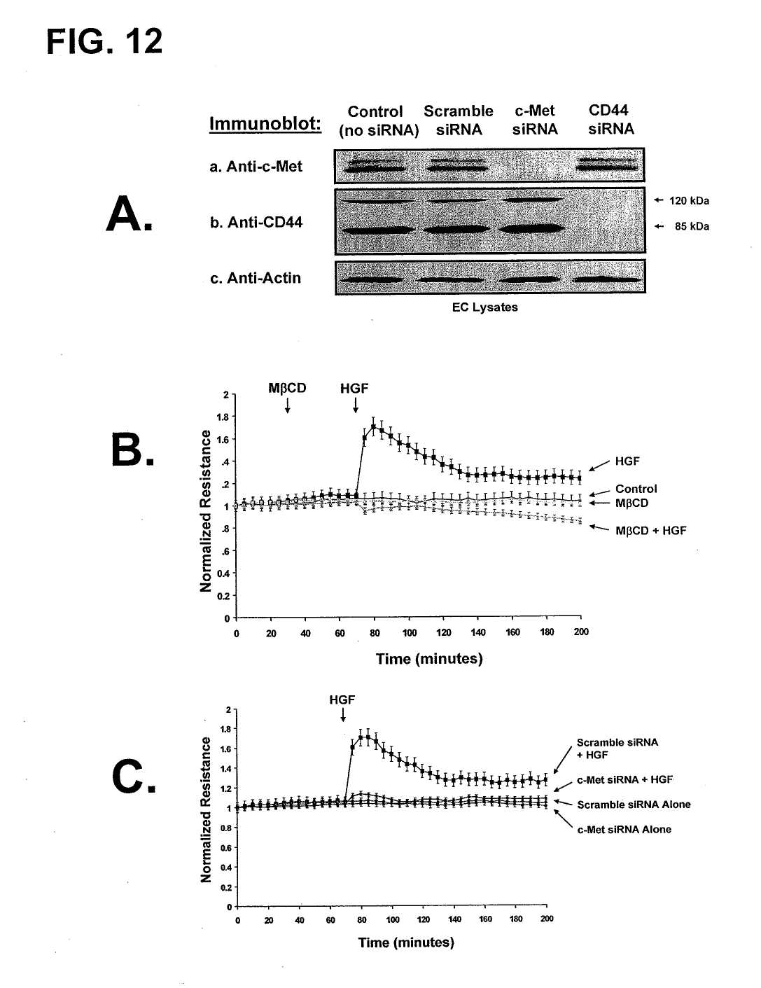

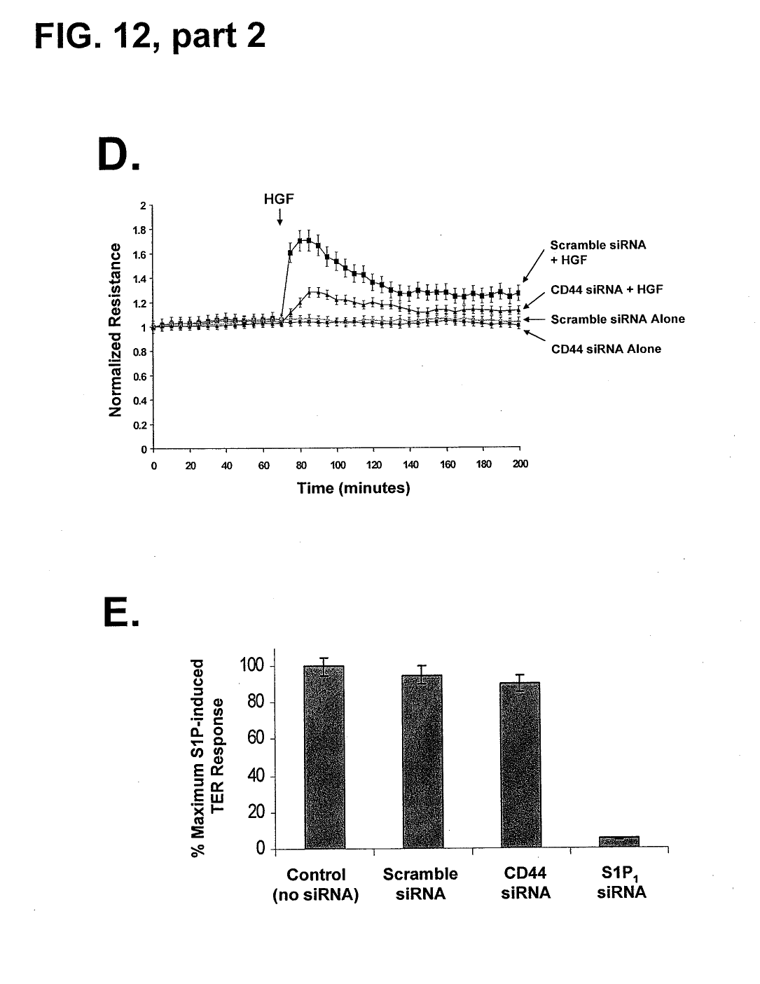

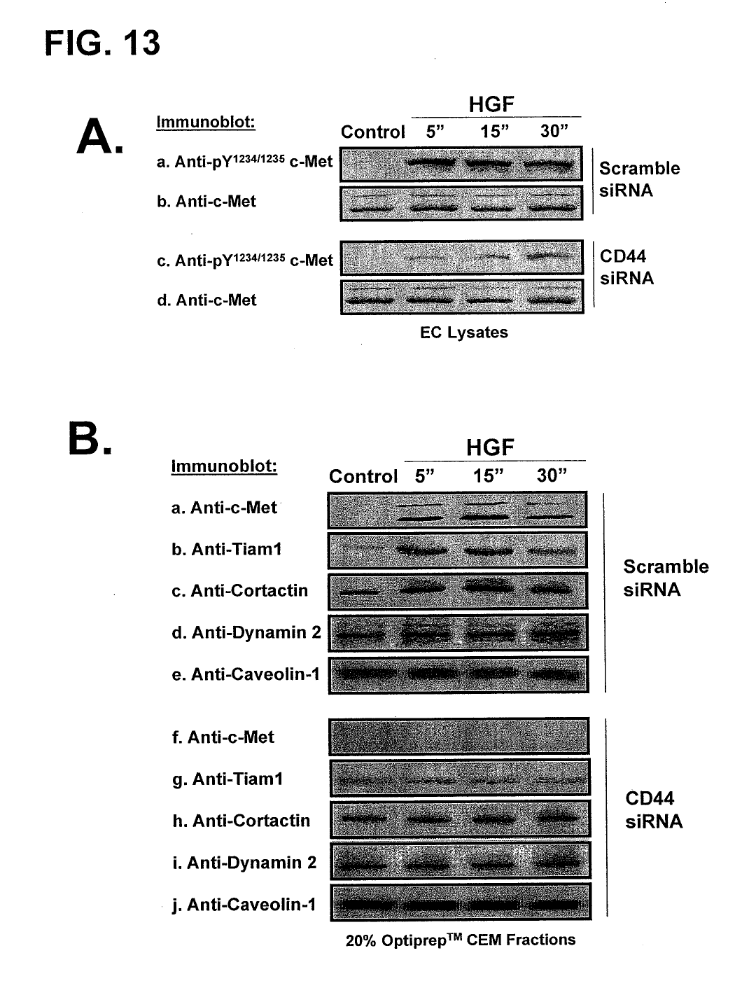

CD44 variant isoforms have been shown to bind HGF (van der Voort et al., 1999) and regulate c-Met autophosphorylation (Tyr1234/1235) (van der Voort et al., 1999; Taher et al., 1999; Orian-Rousseau et al., 2002; Orian-Rousseau et al., 2007), suggesting CD44 can act as a co-receptor for c-Met (Orian-Rousseau et al., 2002; Orian-Rousseau et al., 2007). As shown in FIG. 10-A through 10-C, CD44v10 regulated HGF-mediated c-Met tyrosine phosphorylation (Tyr1234/1235) by ˜50% (FIG. 10-A and -B) and recruitment of c-Met into CEM (FIG. 10-C). As shown in FIG. 11-A, the c-Met recruited to CEM is active (that is, tyrosine phosphorylated). HGF was also shown to induce an association that is time-dependent of c-Met with CD44v10 followed by activation of CD44s and CD44 (defined by CD44 serine phosphorylation) in CEM (see FIGS. 11-B and -C) (Ilangumaran et al., 1999; Tzircotis et al., 2006; Legg et al., 2002; Bourguignon et al., 1999). Eliminating the potential for CEM formation with a plasma membrane cholesterol-depletion agent (methyl-β-cyclodextrin (MβCD)), or reducing the expression of c-Met or CD44 (via siRNA) attenuated HGF-induced increases in human EC barrier function (FIG. 12). These results appeared to be HGF-specific as silencing CD44 expression did not alter the barrier enhancing effects of another CEM-regulated agonist, sphingosine 1-phosphate (S1P) (FIG. 12-E) (Ilangumaran et al., 1998). Moreover, silencing CD44 expression blocked c-Met autophosphorylation (FIG. 13-A). These results strongly suggest an essential role for CD44 and CEM in HGF-induced c-Met activation and EC barrier regulation.

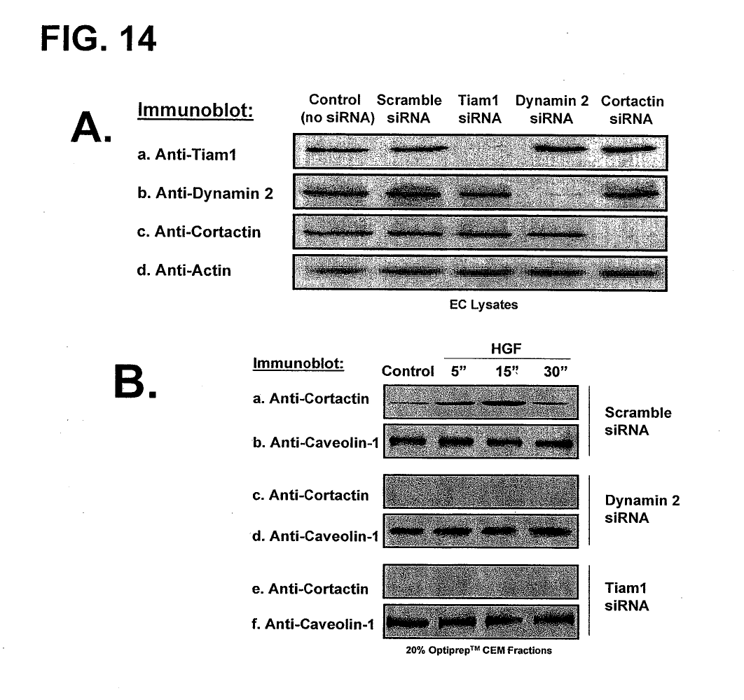

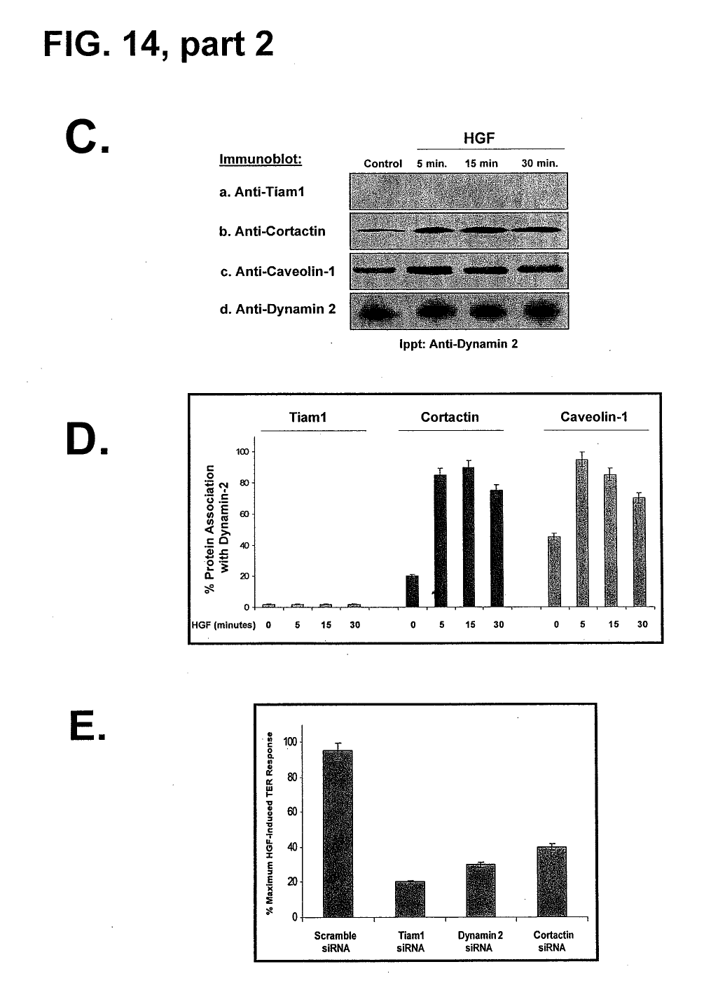

Role of Tiam1, cortactin and dynamin 2 in HGF/c-Met-mediated human EC barrier enhancement. Since CD44 appears to regulate HGF-induced EC barrier enhancement (FIG. 12), whether Tiam1, cortactin and/or dynamin 2 were involved in HGF-induced increases in EC barrier integrity was examined. FIG. 13-B indicates that modest amounts of each of Tiam1, cortactin and dynamin 2 were present within CEM in control EC with increased recruitment to these caveolin-enriched plasma membrane microdomain structures following HGF (25 ng/ml). Silencing CD44 (siRNA) expression attenuated the HGF-induced recruitment of these molecules to CEM (FIG. 13-B); silencing either Tiam1 or dynamin 2 expression abolished cortactin localization to CEM (FIG. 14-A and -B). Immunoprecipitation of dynamin 2 from CEM indicated that cortactin and caveolin-1, but not Tiam1, were complexed with dynamin 2. As shown by FIG. 14-B, HGF-treatment of human EC enhanced this association. Finally, silencing Tiam1, cortactin or dynamin 2 expression attenuated the EC barrier-enhancing effects of HGF (FIG. 14-E), suggesting critical involvement of these molecules in this regard.

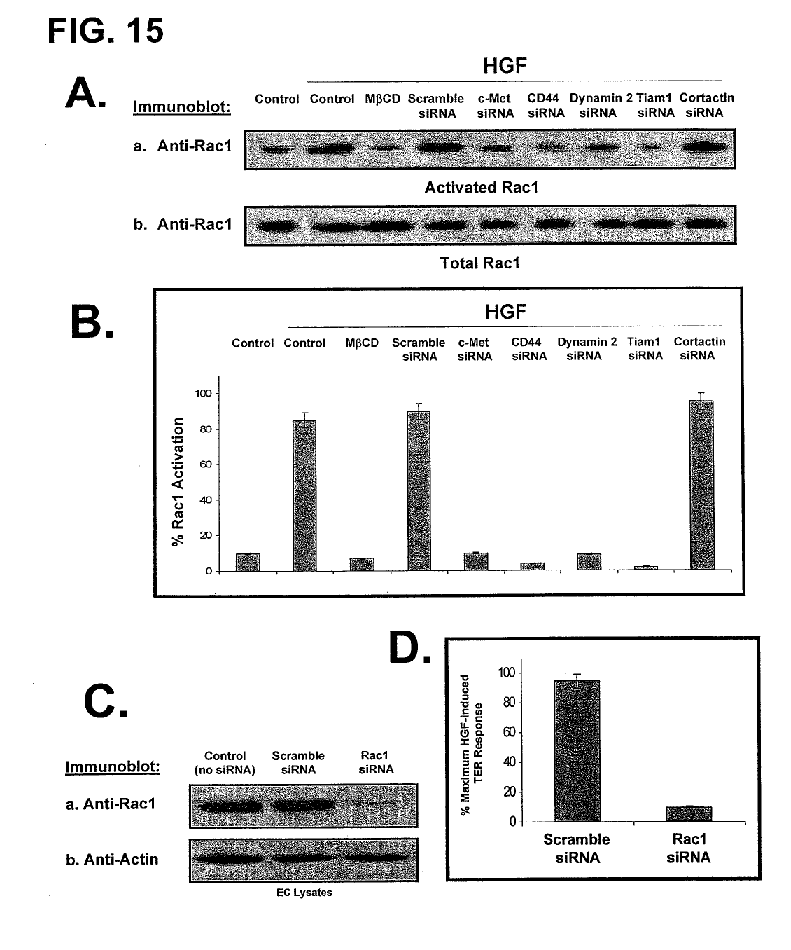

Role of Rac1 in HGF/c-Met-mediated human EC barrier enhancement. The mechanism of HGF-induced Rac1 activation in human EC is poorly defined. As shown in FIG. 15, HGF (25 ng/ml) induced Rac1 activation which is required for HGF-induced human EC barrier enhancement. The inhibition of CEM formation in the presence of methyl-β-cyclodextrin (MβCD), a plasma membrane cholesterol-depletion agent, or silencing (siRNA) c-Met, CD44, Tiam1 or dynamin 2 expression also inhibited HGF-induced Rac1 activation. In contrast, silencing of cortactin expression did not affect HGF-mediated Rac1 activation.

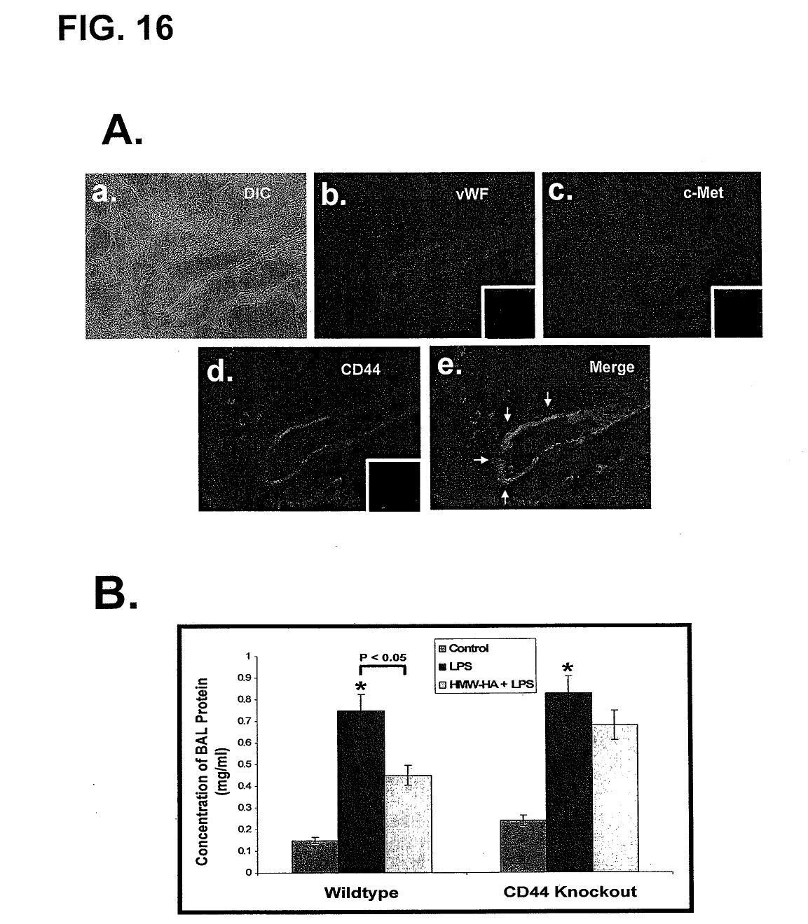

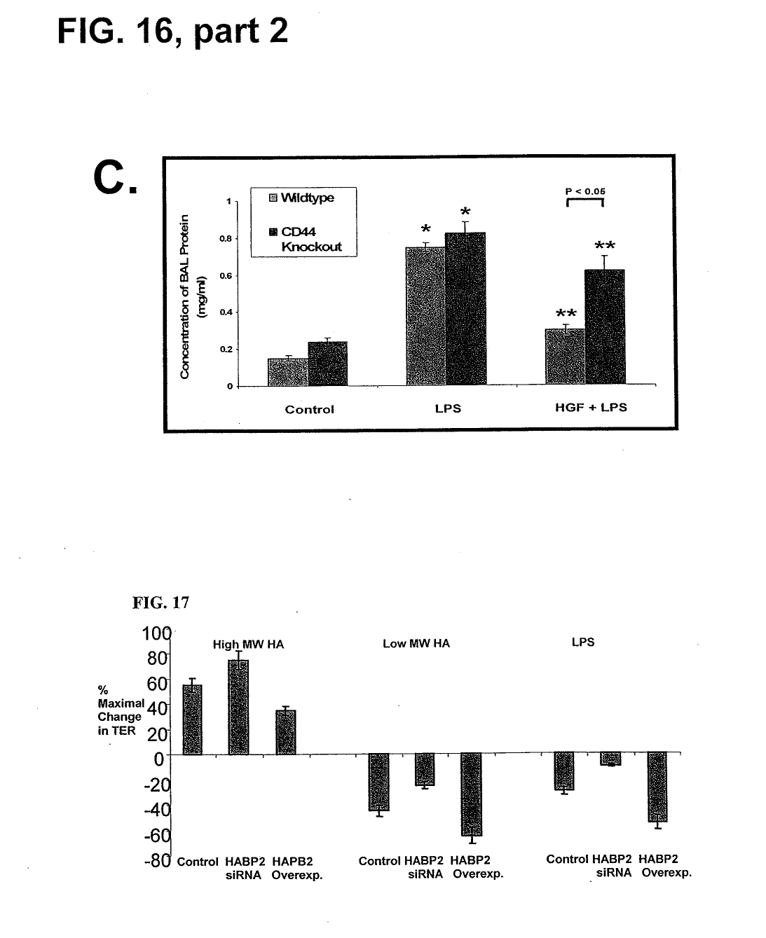

Role of CD44 in HGF-mediated regulation of lung vascular integrity in vivo. Immunohistochemical studies revealed that C57BL/6J wild type murine lung endothelium has colocalized expression of CD44 and c-Met (FIG. 16-A). Whether HGF was an effective barrier protective agent in an in vivo model of lipopolysaccharide (LPS)-induced murine lung vascular permeability was examined next. Intratracheally administered LPS induced murine inflammation and increased vascular leakiness as measured by the protein concentration in bronchioalveolar lavage (BAL) fluid (FIG. 16-B and -C) (Peng et al., 2004). C57BL/6J wildtype mouse pulmonary hyper-permeability was attenuated after intravenous injection of either the CD44 ligand, high molecular weight hyaluronan (HMW-HA, 1.5 mg/kg) (FIG. 16-B) or HGF (50 μg/kg) (FIG. 16-C) four hours after LPS delivery. In contrast, this potent protective effect of both HMW-HA and HGF on LPS-induced inflammatory lung injury was markedly attenuated in the CD44 knockout mouse. This suggests that the protective effect of HGF in LPS-induced pulmonary hyper-permeability is dependent upon CD44 regulation.

In summary, in vitro and in vivo models of pulmonary vascular permeability demonstrate that CD44 regulates HGF-induced vascular integrity via a mechanism that is believed to involve scaffolding of key CEM components (Tiam1, cortactin, dynamin 2 and Rac1) by CD44 isoforms that are essential to the HGF response. These studies indicate that diseases characterized by high permeability states may benefit from HGF therapeutic treatment.

### Example 3

### Role of HABP2 in EC Barrier Function

A. Methods

Cell culture and reagents. Human pulmonary EC were obtained from Cambrex (Walkersville, Md.) and cultured as previously described in EBM-2 complete medium (Cambrex) at 37° C. in a humidified atmosphere of 5% CO₂, 95% air, with passages 6-10 used for experimentation (Garcia et al. 2001). Unless otherwise specified, reagents were obtained from Sigma. Reagents for SDS-PAGE electrophoresis were purchased from Bio-Rad. Rat anti-CD44 (IM-7, common domain) antibody was purchased from BD Biosciences (San Diego, Calif.). Mouse anti-actin antibody and lipopolysaccharide (LPS) was purchased from Sigma. Secondary horseradish peroxidase-labeled antibodies were purchased from Amersham Biosciences (Piscataway, N.J.). Mouse lung homogenates were obtained by solubilizing extracted lungs in solubilization buffer (50 mM HEPES (pH 7.5), 150 mM NaCl, 20 mM MgCl₂, 1% Triton X-100, 0.2% SDS, 0.4 mM Na₃VO₄, 40 mM NaF, 50 μM okadaic acid, 0.2 mM phenylmethylsulfonyl fluoride, 1:250 dilution of Calbiochem protease inhibitor mixture 3) with sonication. HABP2 overexpression plasmids were purchased from Origene. Purified HABP2 protein was obtained by overexpression of HABP2 plasmid in human pulmonary EC, collection of EC media, and immunoprecipitation with anti-HABP2 antibody. The HABP2 was eluted from the immunobleads in 1M NaCl with 0.1% NP-40. The eluted purified HABP2 was dialyzed against 0.05 M sodium borate buffer, pH=8.5, for 24 h at 4° C. in 500-Da cutoff Spectra-Por tubing (Pierce-Warriner, Chester, UK). The concentration and purity of the purified HABP2 protein were analyzed using Bio-Rad DC Protein Assay kit II and running sample on SDS-PAGE and either staining with Imperial™ protein stain (Pierce) or immunoblotting with anti-HABP2 antibody. Anti-HABP2 antibody was purchased from Novus Biologicals. Anti-Hyal-1, 2, 3, 4 antibodies, Ant-PAI-1, -2 antibodies, Anti-fibronectin, anti-vitronectin, anti-tenascin, and anti-perlecan antibodies were purchased from Santa Cruz Biotechnology.

Measurement of transendothelial monolayer electrical resistance (TER). EC were grown to confluence in polycarbonate wells containing evaporated gold microelectrodes, and TER measurements performed using an electrical cell-substrate impedance sensing system obtained from Applied Biophysics (Troy, N.Y.) as previously described in detail (Garcia et al. 2001). TER values from each microelectrode were pooled at discrete time points and plotted versus time as the mean±S.E.

Immunoblotting. Proteins in the EC lysates or mouse lung homogenates were solubilized in solubilization buffer, separated on SDS-PAGE, and analyzed by immunoblotting with specific antisera and HRP conjugated secondary antibody with enhanced chemiluminescence detection (Amersham Biosciences).

Treatment with LPS, low and high MW hyaluronan. EC were treated with 1.0 μg/ml LPS, 100 nM HMW-HA (˜1 million Daltons) or 100 nM LMW-HA (˜2,500 Daltons) for 24 hours and EC media and lysates were obtained.

HABP2 protease assay. Protease activity is measured using the QuantiCleave™ Protease Assay Kit (Pierce). Briefly, the immunobeads are incubated with succinylated casein for one hour followed by development with TNBSA (2,4,6-trinitrobenzene sulfonic acid) and read at 450 nm.

Preparation and quantitation of low and high MW hyaluronan (HA)—The method of preparation is similar to that described previously (Slevin et al., 2002). For HMW-HA, 500 mg of rooster comb HA 1-million Da polymers (Bourguignon et. al. 2004) was dissolved in distilled water and centrifuged in an Ultrafree-MC™ Millipore 100,000 Da MW cutoff filter and the flow-through (less than 100,000 Da) was discarded. For LMW-HA, 500 mg of rooster comb HA was digested with 20,000 units of bovine testicular hyaluronidase in digestion buffer (0.1 M sodium acetate, pH 5.4, 0.15 M NaCl) for 24 h, and the reaction stopped with 10% trichloroacetic acid. The resulting solution was centrifuged in an Ultrafree-MC™ Millipore 5,000 Da MW cutoff filter and the flow-through (less than 5,000 Da) was dialyzed against distilled water for 24 h at 4° C. in 500-Da cutoff Spectra-Por tubing (Pierce-Warriner, Chester, UK). Low and High MW HA were quantitated using an ELISA-like competitive binding assay with a known amount of fixed HA and biotintylated HA-binding peptide (HABP) as the indicator (Pogrel et. al., 2003). In some cases, both Low and High MW HA were subject to boiling, proteinase K (50 μg/ml) digestion, hyaluronidase SD digestion (100 milliunits/ml) or addition of boiled (inactivated) hyaluronidase SD to test for possible protein/lipid contaminants (Calabro et. al., 2000). LMW and HMW-HA with DNA standards were run on 4-20% SDS-PAGE gels and stained with combined Alcian blue and silver staining to further determine HA purity and size (Min and Cowman, 1986).

Construction and transfection of siRNA against HABP2—The siRNA sequence(s) targeting against HABP2 were generated using mRNA sequences from GenBank™. HABP2 siRNA (gi: 20302151) and a scramble sequence which does not target any known human mRNA sequence were utilized. For HABP2 mRNA (or scramble), two targets were identified. Specifically, HABP2 target sequence 1 (5′-AAAGGCATAGACAACAAAAGA-3′ (SEQ ID NO:35)), HABP2 target sequence 2 (5′-AACAAAAGAAATTTTATTGAG-3′ (SEQ ID NO:36)), scrambled sequence 1 (5′-AAGAGAAATCGAAACCGAAAA-3′ (SEQ ID NO:24)) and scramble sequence 2 (5′-AAGAACCCAATTAAGCGCAAG-3′ (SEQ ID NO:25)) were utilized. For construction of the siRNA, a transcription-based kit from Ambion was used (Silencer™ siRNA construction kit). Human lung EC were then transfected with siRNA using siPORTamine™ as the transfection reagent (Ambion, Tex.) according to the protocol provided by Ambion. Cells 40% confluent) were serum-starved for 1 h followed by incubated with 3 μM (1.5 μM of each siRNA) of target siRNA (or scramble siRNA or no siRNA) for 6 h in serum-free media. The serum-containing media was then added (1% serum final concentration) for 42 h before biochemical experiments and/or functional assays were conducted.

Statistical analysis. Student's t test was used to compare the means of data from two or more different experimental groups. Results are expressed as means±S.E.

B. Results

Divergent effects of LPS, low and high molecular weight hyaluronan on HABP2 protein expression and activity. To test whether HAPB2 protein expression is regulated by hyaluronan, endogenous HABP2 protein levels were compared in endothelial cell (EC) lysates and mouse lung homogenates under different conditions by immunoblotting. HABP2 protein level was significantly enhanced in EC lysates treated with lipopolysaccharide (LPS), low molecular weight (MW) hyaluronan and their combination, but reduced when treated with high MW hyaluronan and the combination of high MW hyaluronan and LPS; LPS also increased HABP2 expression in mouse lung homogenates. Actin expression was used as loading control. Likewise, adding of low MW hyaluronan increased the protease activity of purified HABP2 in a dose-dependent manner and adding of high MW hyaluronan reduced its activity.

HABP2 regulates hyaluronan- and LPS-induced EC barrier function. The inventors contemplated whether HABP2 contributes to EC barrier function, which involves hyaluronan and LPS regulation. High MW hyaluronan increased transendothelial monolayer electrical resistance (TER), whereas low MW hyaluronan and LPS induced negative TER changes ultimately resulting in EC barrier disruption. Silencing of HABP2 expression promoted the EC barrier enhancing effects of high MW hyaluronan and consistently overexpression of HABP2 blocked these effects. HABP2 silencing also reduced the effects of low MW hyaluronan and LPS on EC barrier disruption while HABP2 overexpression enhanced these effects (FIG. 17).

Role of HABP2 in regulation of CD44 and hyaluronidase expression. As shown above, low MW hyaluronan stimulates HABP2 protease activity. It is known that LPS stimulates production of low MW hyaluronan in EC via degradation of high MW hyaluronan by hyaluronidases and low MW hyaluronan binds to CD44v10. To study the effects of HABP2 on CD44 and hyaluronidase expression, CD44 and hyaluronidase protein levels were compared by immunoblotting in human EC cell lysates transfected with scramble siRNA and HABP2 siRNA. HABP2 silencing of protein expression was confirmed by HABP2 immunoblotting in the lower panel. After normalization by actin protein levels, HABP2 silencing did not appear to regulate CD44 expression in EC. CD44 expression was upregulated in lungs with LPS. Hyaluronidase variants Hyal2 and Hyal3 appeared to be differentially regulated by LPS, hyaluronan, and HABP2 siRNA.

Regulation of plasminogen activator inhibitor 1 (PAI-1) degration by LPS and HABP2. To study the effects of HABP2, LPS and hyaluronan on PAI expression, PAI-1 and PAI-2 protein levels were compared by immunoblotting in human EC cell lysates transfected with scramble siRNA and HABP2 siRNA. Degraded PAI-1 was in LPS-induced EC transfected with scrambled siRNA but not in those with HABP2 siRNA; PAI-2 did not change significantly under those conditions.

Regulation of ECM proteins by HABP2. The effects of HABP2, LPS and hyaluronan on expression of endothelial extracellular matrix (ECM) proteins, such as fibronectin, vitronectin, tenascin, and perlecan were studied by immunoblotting in human EC cell lysates transfected with scramble siRNA and HABP2 siRNA. Tenascin and perlecan appeared to be upregulated in EC transfected with HABP2 siRNA while fibronectin and vitronectin appeared to have no changes in expression.

### Example 4

### Role of CD44 and caveolin-1 in Hyaluronan and LPS-Mediated Lung Function

A. Methods

Animals. Male C57BL/6J mice, CD44 knockout mice, Caveolin-1 knockout mice (8-10 weeks, Jackson Laboratories, Bar Harbor, Me.) were anesthetized with intraperitoneal ketamine (150 mg/kg) and acetylpromazine (15 mg/kg) according to approved protocols. LPS (2.5 mg/kg) or saline (control) were instilled intratracheally and four hours later, HMW-HA (1.5 mg/kg) or saline control delivered intravenously through the internal jugular vein. The animals were allowed to recover for 24 hours followed by bronchioalveolar lavage protein analysis and/or lung immunohistochemistry.

Determination of protein concentration. Total protein concentration. TGF-alpha concentration. TGF-beta1 concentration. Bronchioalveolar lavage (BAL) was performed by an intratracheal injection of 1 cc of Hank's balanced salt solution followed by gentle aspiration. The recovered fluid was processed for protein concentration (BCA Protein Assay Kit; Pierce Chemical Co., Rockford, Ill.) or used to determine TNF-α and TGF-β1 concentrations using quantitative sandwich enzyme immunoassays (R & D Systems, Minneapolis, Minn.).

Statistical analysis. Student's t test was used to compare the means of data from two or more different experimental groups. Results are expressed as means±S.E.

B. Results

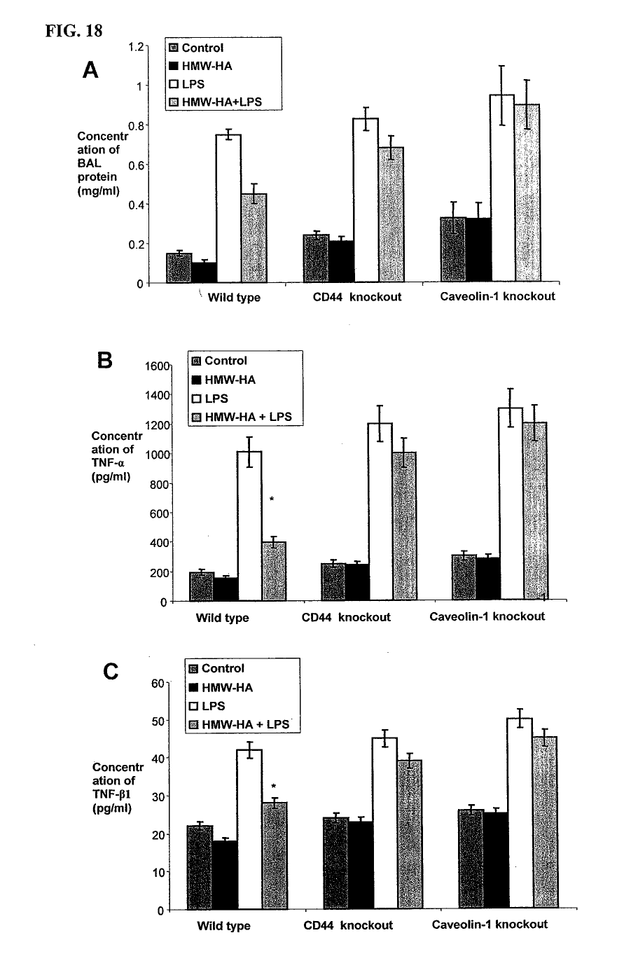

Male C57BL/6J, CD44 knockout and Caveolin-1 knockout mice were anesthetized and were either given saline (control) or LPS (2.5 mg/kg) intratracheally. After 4 hours, mice were given intravenously injections (internal jugular vein) with saline (control) or high molecular weight hyaluronan (HMW-HA, 1.5 mg/kg). The treated mice were allowed to recover for 24 hours, bronchioalveolar lavage (BAL) fluids were obtained and concentrations of total protein, TGF-alpha, TGF-beta1 were determined. N=6 per condition with the single asterisk (*) referring to a significant (p<0.05) difference between control and LPS treatment. High MW hyaluronan reduced the enhancing effect of LPS on BAL protein concentration and also TGF-alpha and TGF-beta1 concentration in BAL fluids of wild type mice, but not in CD44 knockout and Caveolin-1 knockout mice (FIG. 18).

### Example 5

### Hyaluronan/CD44 Regulation of Pulmonary Vascular Permeability

A. Methods

Cell culture and reagents. Human pulmonary EC were obtained from Cambrex (Walkersville, Md.) and cultured as previously described in EBM-2 complete medium (Cambrex) at 37° C. in a humidified atmosphere of 5% CO₂, 95% air, with passages 6-10 used for experimentation (Garcia et. al., 2001). Unless otherwise specified, reagents were obtained from Sigma.

Preparation, Quantitation and Fluorescent-labeling of High MW Hyaluronan (HA). The method of preparation was described in detail previously (Singleton et. al., 2006). In some cases, High MW HA was subject to boiling or proteinase K (50 ug/ml) digestion. The method for fluorescent labeling of HA was previously discussed in detail (Seyfried et. al., 2005).

Lipid raft Isolation. Caveolin-enriched microdomains known as lipid rafts were isolated from human lung EC as previously described (Singleton et. al., 2005, 2006). Triton X-100-insoluble materials were mixed with 0.6 ml of cold 60% Optiprep™ and overlaid with 0.6 ml of 40%-20% Optiprep™ and the gradients centrifuged (35,000 rpm) in SW60 rotor for 12 h at 4° C. and different fractions were collected and analyzed. In some cases, different fractions were analyzed for total cholesterol content using the Amplex Red™ cholesterol assay kit.

Immunoprecipitation and Immunoblotting. Cellular materials associated within the 20% Optiprep™ fractions (CEM fraction) were incubated with IP buffer (50 mM HEPES (pH 7.5), 150 mM NaCl, 20 mM MgCl₂, 1% Triton X-100, 0.1% SDS, 0.4 mM Na₃VO₄, 40 mM NaF, 50 μM okadaic acid, 0.2 mM phenylmethylsulfonyl fluoride, 1:250 dilution of Calbiochem protease inhibitor mixture 3). The samples were then immunoprecipitated with specific antibodies followed by SDS-PAGE in 4-15% polyacrylamide gels, transfer onto Immobilon™ membranes, and developed with specific primary and secondary antibodies. Visualization of immunoreactive bands was achieved using enhanced chemiluminescence (Amersham Biosciences) as described previously (Singleton et. al., 2005, 2006).

Construction and Transfection of siRNA. Target sequences for siRNA were generated by scanning the target gene and identifying unique 19 nucleotide unique sequences. Sense and antisense DNA 29 oligonucleotide (21 nucleotides encoding the siRNA, 8 nucleotides encoding a T7 promoter primer were generated against identified target sequences. Then, the double stranded RNA were made and transfected into human pulmonary EC at a concentration of 10 nM using Ambion siRNA transfection reagent. A scramble sequence that does not have any known gene target was transfected as a control. Verification of siRNA efficiency was determined using immunoblotting with a specific antibody. Immunoblotting with antibodies against non-target proteins (including actin) was used to determine the specificity of the siRNA.

Rac1 Activation Assay. Rac1 activities in human lung EC were performed as described previously (Singleton et al., 2005).

Caveolin-1-GFP Construction, Transfection and Live Cell Imaging. This reagent and procedures were previously described in detail (Volonte et. al., 1999, Parat et. al., 2003).

Atomic Force Microscopy. The protocols/procedures for AFM analysis of 50 to 100 nm pits (CEM, lipid rafts), EC height and force measurements were previously described in detail (Dvorak, 2003, Reich et al., 2001, Henderson et al., 2004, Milhiet et. al., 2004, Miklaszewska, 2004, Lucius, 2003). EC treated with various siRNAs or treated with MβCD with or without 100 nM high MW HA (0, 15, 30, 60 min.) were fixed in 4% glutaraldehyde and analyzed in tapping mode on a DI multimode AFM (Digital Instruments, Santa Barbara, Calif.) using a V-shaped oxide-sharpened silicon nitride cantilever with an optical scanning speed of ˜1.0 Hz to obtain a surface topology map which was quantitated and spatially defined using Metamorph™ software. For live cells, GFP and GFP-caveolin-1 expressing EC were treated with or without 100 nM high MW HA in a DI Bioscope cell chamber (Digital Instruments, Santa Barbara, Calif.) were exposed to tapping mode AFM with concurrent fluorescence scanning and images were obtained using a C5985 chilled CCD camera (Hamamatsu Photonics Systems, Bridgewater, N.J.).

Measurement of TransEC Electrical Resistance (TER). EC were grown to confluence in polycarbonate wells containing evaporated gold microelectrodes, and TER measurements performed using an electrical cell-substrate impedance sensing system (Applied Biophysics, Troy, N.Y.) as previously described (Garcia et al., 2001). TER values from each microelectrode were pooled at discrete time points and plotted versus time as the mean±S.E.

Immunofluorescence Microscopy. EC were serum-starved for one hour prior to the addition of 100 nM high MW HA. EC were then fixed in 4% paraformaldehyde, permeablized with ethanol and incubated with specific protein or interest primary antibody for 30 min followed by either FITC or Texas Red-conjugated secondary (Invitrogen (Molecular Probes), Eugene, Oreg.) and analyzed using a Nikon Eclipse TE 300 microscope as described (Garcia et. al., 2001).

Animal Preparation and Treatment. Male C57BL/6J, B6129N2, CD44^{-/-} or caveolin-1^{-/-} mice (8-10 weeks, Jackson Laboratories, Bar Harbor, Me.) were anesthetized with intraperitoneal ketamine (150 mg/kg) and acetylpromazine (15 mg/kg) before exposure of the right internal jugular vein via neck incision. LPS (2.5 mg/kg) or water (control) were instilled intratracheally. Four hours later, mice receive high MW HA (1.5 mg/kg) or water control through the internal jugular vein. The animals were allowed to recover for 24 hours after LPS before bronchioalveolar lavage protein and cytokine concentration and/or lung immunohistochemistry/immunoblot analysis (Peng et al., 2004).

Mouse Lung Immunohistochemistry. To characterize the expression of proteins in mouse lung vascular endothelial cells (EC), lungs from mice were formalin fixed, 5 micron paraffin sections were obtained, hydrated and epitope retrieval was performed (DakoCytomation Target Retrieval Solution, pH=6.0, DakoCytomation, Carpinteria, Calif.) The sections were then histologically evaluated by specific primary antibody and secondary HRP labeled polymer with DAB staining (Dako EnVision™+System, HRP (DAB) (DakoCytomation, Carpinteria, Calif.)) followed by hematoxylin QS counterstaining (Vector Laboratories, Burlingame, Calif.). Negative controls for immunohistochemical analysis were done by the same method as above but without primary antibody. Immunostained sections were photographed using a Leica Axioscope.

Determination of Bronchoavleolar Lavage Protein and Cytokine Concentration. Bronchioalveolar lavage (BAL) was performed by an intratracheal injection of 1 cc of Hank's balanced salt solution followed by gentle aspiration. The recovered fluid was processed for protein concentration (BCA Protein Assay Kit; Pierce Chemical Co., Rockford, Ill.) (Peng et. al., 2004). Cytokines (IL-6, TNFalpha) were measured using a Quantikine sandwich ELISA kit (R & D Systems).

Statistical analysis. Student's t test was used to compare the means of data from two or more different experimental groups. Results were expressed as means±S.E.

B. Results

Analysis of reproducibility of lipid raft (caveolin-enriched microdomains) isolations from control and HA-treated human pulmonary EC. Caveolin-enriched microdomains (CEM) from control or HA (100 ng/ml, 15 min.) treated human pulmonary EC were isolated and subjected to 1D or 2D SDS-PAGE followed by protein staining with Imperial Blue. These studies demonstrated reproducibility in protein staining with various lipid raft (CEM) isolation preparations.

Colocalization of CD44 and high molecular weight HA with caveolin-1 in human pulmonary EC. Fluorescent microscopy indicated that high MW HA and CD44 colocalized with caveolin-1 in discreet punctuate cytosolic structures and at the EC periphery.

Expression of GFP-caveolin-1 in human pulmonary EC. Caveolin-1 is a marker for lipid rafts (CEM) (Drab et. al., 2001). Successful expression of GFP-caveolin-1 is required to visualize CEM movement in real time and to perform combined fluorescence/AFM on living EC. GFP-caveolin-1 was expressed in human pulmonary EC and showed discreet punctuate cytosolic and peripheral membrane localization, similar to endogenous caveolin-1 staining.

Characterization of high MW HA-induced human pulmonary EC morphological changes and CEM dynamics using Atomic Force Microscopy (AFM). The 50 to 100 nM structures previously identified as lipid rafts were sensitive to cholesterol depletion by MβCD. Treatment with high MW HA caused dramatic morphological changes including increased thickening of EC junctions. Further, the total number of lipid raft-like structures did not change with high MW HA addition. However, recruitment of lipid rafts to the EC junctions suggested a role for lipid rafts in high MW-HA-mediated EC junctional regulation.

Characterization of HA/CD44 in lipid rafts or CEM effects on human pulmonary EC barrier function. High MW HA induces a dose-dependent increase in TER while low MW HA induces a biphasic response ultimately leading to EC barrier disruption. The HMW-HA-induced increase in TER reached plateau after ˜3 hours and remained sustained for several hours. HA purity (i.e. there were no contaminating proteins in the preparation) was indicated maximal HA TER response with boiling or proteinase digestion controls. The effects of HA on TER were inhibited by abolishing lipid rafts with MβCD which depletes cholesterol from the plasma membrane or blocking CD44 with an antibody that binds to the HA binding site of all CD44 isoforms and blocks HA binding (IM-7 antibody).

Analysis of HA and CEM-mediated actin cytoskeletal reorganization in human EC. EC were serum-starved for one hour and were either untreated (control), treated with 100 nM High MW HA for 30 min. or treated with MβCD for one hour prior to HA addition. EC were then probed with TRITC-phalloidin. HMW-HA induced cortical actin reorganization was inhibited by abolishing CEM formation (MβCD).

Immunoblot analysis of siRNA downregulation of CD44, Caveolin-1, Tiam1, Dynamin 2 and Rac1 expression in human pulmonary EC. In order to demonstrate the contributions of caveolin-1, Dynamin 2, PI3 kinase/AKT and Tiam1 on HA-mediated CD44 and CEM-dependent Rac1 activation and consequent EC barrier function, siRNA was designed and tested against these target molecules. CD44 siRNA targets all isoforms. These results demonstrate effective silencing of these molecules.

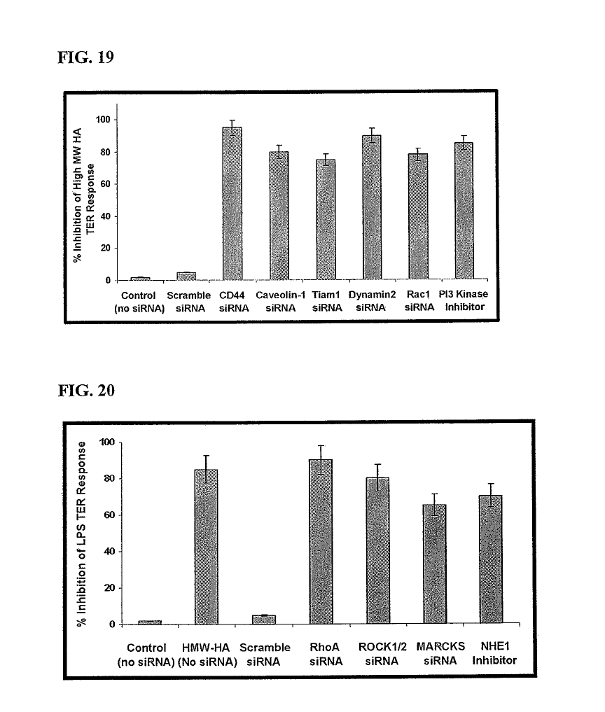

Effects of siRNA downregulation and specific inhibitors on high MW HA-mediated increased human pulmonary EC barrier enhancement. CD44, caveolin-1, Tiam1, Dynamin2, Rac1 and the PI3 kinase pathway each substantially contribute to regulating high MW HA-mediated EC barrier enhancement as indicated in FIG. 19.

Immunoblot analysis of siRNA downregulation of RhoA, ROCK 1/2 and MARCKS expression in human pulmonary EC. In order to demonstrate the role of HA/CD44 inhibition of lipopolysaccharide (LPS)-induced ROCK-mediated phosphorylation of MARCKS and NHE1 in CEM leading to EC barrier disruption, siRNA was designed and tested against these target molecules. These results demonstrate effective silencing of these molecules.

Effects of silencing RhoA, ROCK 1/2, MARCKS and inhibiting NHE1 on LIPS-induced EC barrier disruption. At a concentration of 1 LPS induced a delayed EC barrier disruptive response (starting at ˜4 hours) similar to that observed in vivo (Peng et. al., 2004). FIG. 20 indicates that HMW-HA (100 ng/ml) protects from LPS-induced EC barrier disruption. RhoA, ROCK1/2, MARCKS and NHE1 all had a substantial effect on regulating LPS-mediated EC barrier disruption.

High MW HA and the ROCK inhibitor, Y-27632, inhibit LPS-induced phosphorylation of MARCKS and NHE1 in human pulmonary EC. Inhibition of ROCK attenuates LPS-induced acute lung injury (Tasaka et al., 2005). Two downstream targets of ROCK in lipid rafts (CEM) that can potentially regulate LPS-induced EC barrier disruption include the actin and phospholipid binding protein Myristoylated alanine-rich C-kinase substrate (MARCKS) (Tanabe et al., 2006, Noma et al., 2006, Ikenoya et al., 2002) and the sodium-hydrogen exchanger 1 (NHE1) (Bourguignon et. al., 2004). LPS can induce MARCKS serine phosphorylation (Zhao and Davis, 2000). Phosphorylation of MARCKS inhibits its association with the plasma membrane and promotes cytosolic localization (Aderem, 1995, Matsubara, 2005, Sundaram et. al., 2004). LPS can also regulate NHE1 (Cetin et al., 2004). Serine/threonine phosphorylation of NHE1 in CEM promotes hyaluronan (HA) degradation (Bourguignon et al., 2004). To test the effect of high MW-HA and the ROCK inhibitor in LPS-induced phosphorylation of MARCKS and NHE1 in human pulmonary EC, confluent EC were either untreated (control), treated with LPS (1 μg/ml, 4 hours), LPS (1 μg/ml, 4 hours)+HMW-HA (100 nM, 4 hours) or LPS (1 μg/ml, 4 hours)+Y-27632 (500 nM, 4 hours), solubilized and immunoprecipitated with anti-MARCKS (A) or anti-NHE1 (B) antibody. The immunoprecipitated material was run on SDS-PAGE and immunoblotted with anti-phospho-Serine (A-a, B-a), anti-phospho-Threonine (A-b, B-b), anti-MARCKS (A-c) or anti-NHE1 (B-c) antibody. The results showed that high MW HA and the ROCK inhibitor, Y-27632, inhibit LPS-induced phosphorylation of MARCKS and NHE1 in human pulmonary EC.

Immunoblot analysis of CD44 and Caveolin-1 expression in control, LPS- and HA-treated mouse lung homogenates. CD44 is highly likely to be important in lung disease as CD44−/− mice develop lung fibrosis, inflammatory cell recruitment and accumulation of hyaluronan fragments at sites of lung injury (Teder et al., 2002). CD44 expression can be upregulated by LPS in certain cell types (Weiss et al., 1998). Caveolin-1 can be differentially regulated in various models of acute lung injury (ALI). Monocrotaline-induced rodent pulmonary hypertension results in a loss of lung EC caveolin-1 expression (Mathew et al., 2004). In contrast, there is no change in pulmonary caveolin-1 expression in lipopolyscaaharide (LPS)-induced sepsis with lung injury (Garrean et al., 2006). Therefore, the expression of CD44 and caveolin-1 was examined in control, LPS challenged and LPS-challenged followed by HMW-HA treated C57BL/6J mouse lungs. The results indicate that LPS upregulates CD44 expression (˜85 kDa, 116 kDa, ˜200 kDa) using a pan-CD44 antibody which recognizes all CD44 isoforms (IM-7) in mouse lungs. Further, HMW-HA inhibited LPS-induced CD44 upregulation (HMW-HA alone had no effect on CD44 expression). In contrast, LPS or HMW-HA+LPS did not significantly alter caveolin-1 expression.

Immunohistochemical analysis of CD44 staining in control and LPS-treated mouse lung vasculature. Since CD44 can be expressed in a variety of cell types including neutrophils, the effects of LPS challenge on CD44 expression were examined in the mouse lung vasculature. CD44 immunostaining increased after LPS challenge in pulmonary EC and the surrounding vasculature.

High MW HA protects from LPS-induced vascular hyper-permeability in mice. LPS induced vascular leakiness associated with increased total protein in the bronchioalveolar lavage (BAL) fluid of mouse lungs. However, intravenous administration of high MW HA four hours after LPS attenuated the vascular hyper-permeability.

High MW HA protection from LPS-induced vascular hyper-permeability is inhibited in CD44 and Caveolin-1 knockout mice. HMW-HA did not protect from LPS-induced ALI in CD44 and Caveolin-1 knockout mice, indicating an essential role for CD44 and CEM in the HMW-HA protective response.

### Example 6

### HABP2/C1INH Regulation of Acute Lung Injury

A. Methods

Cell culture preparation and treatment. Human pulmonary microvascular EC (HPMVEC) (Cambrex), grown in EBM-2 complete medium (Cambrex) at 37° C. in a humidified atmosphere of 5% CO2, 95% air, with passages 6-10 used for experimentation will be treated with or without 1.0 μg/ml LPS, 100 nM HMW-HA and/or 100 nM LMW-HA in the presence or absence of exogenous purified recombinant HABP2 polyanion binding domain and/or C1INH (Novus Biologicals), HABP2 and/or C1INH overexpression vector or siRNA (scramble, HABP2, C1INH, PAR-1, PAR-2, PAR-3, PAR-4, tenascin-C or perlecan) or the potent hyaluronidase inhibitor, L-ascorbic acid 6-hexadecanoate (Botzki et al., 2004) (Sigma). Extracellular media and/or treated EC will either be analyzed for protein expression, HABP2 protease activity, hyaluronidase activity or Trans-endothelial Electrical Resistance (TER). Cellular lysates were obtained with lysis buffer (50 mM HEPES (pH 7.5), 150 mM NaCl, 20 mM MgCl2, 1% Triton X-100, 0.1% SDS, 0.4 mM Na3VO4, 40 mM NaF, 50 μM okadaic acid, 0.2 mM phenylmethylsulfonyl fluoride, 1:250 dilution of Calbiochem protease inhibitor mixture 3). The samples were then run on SDS-PAGE in 4-15% polyacrylamide gels, transfer onto Immobilon™ membranes, and developed with specific primary (i.e. anti-HABP2 (Novus Biologicals), PAR-1, PAR-2, PAR-3, PAR-4, tenascin-C or perlecan (Sana Cruz Biotechnology) antibody) and secondary antibodies. Visualization of immunoreactive bands were achieved using enhanced chemiluminescence (Amersham Biosciences) as the inventors have described previously.

Construction and Transfection of siRNA. Target sequences for siRNA were generated by scanning the target gene and identifying unique 19 nucleotide unique sequences. Sense and antisense DNA 29 oligonucleotide (21 nucleotides encoding the siRNA, 8 nucleotides encoding a T7 promoter primer, were generated against identified target sequences. Then, the double stranded RNA were made and transfected into human pulmonary EC at a concentration of 10 nM using Ambion siRNA transfection reagent. A scramble sequence that does not have any known gene target was transfected as a control. Verification of siRNA efficiency was determined using immunoblotting with a specific antibody. Immunoblotting with antibodies against non-target proteins (including actin) was used to determine the specificity of the siRNA.

Determination of EC barrier function. Human pulmonary endothelial cells were grown to confluence in polycarbonate wells containing evaporated gold microelectrodes (surface area, 10-3 cm²) in series with a large gold counter electrode (1 cm²) connected to a phase-sensitive lock-in amplifier as described previously. Measurements of transendothelial electrical resistance (TER) were performed using an electrical cell-substrate impedance sensing system (ECIS) (Applied BioPhysics Inc.). Briefly, current was applied across the electrodes by a 4,000-Hz AC voltage source with amplitude of 1 V in series with a 1 MS/resistance to approximate a constant current source (˜1 μA). The in-phase and out-of-phase voltages between the electrodes were monitored in real time with the lock-in amplifier and subsequently converted to scalar measurements of transendothelial impedance, of which resistance was the primary focus. TER was monitored for 30 minutes to establish a baseline resistance (R0) which, for pulmonary endothelium, was typically between 8 to 12×10³Ω (wells with R0<7×10³Ω were rejected). As cells adhere and spread out on the microelectrode, TER increases (maximal at confluence), whereas cell retraction, rounding, or loss of adhesion was reflected by a decrease in TER. These measurements provide a highly sensitive biophysical assay that indicates the state of cell shape and focal adhesion. Values from each microelectrode were pooled at discrete time points and plotted versus time as the mean±SE of the mean (Garcia et al., 2001).

Hyaluronidase enzymatic assay. Cellular lysates were immunoprecipitated with either anti-Hyal1, anti-Hyal-2, anti-Hyal-3 or anti-Hyal-4 antibody (Santa Cruz Biotechnology) followed by secondary antibody-conjugated Sepharose beads. Biotinylated HA covalently bound to Sepharose beads with the aid of 1-ethyl-3-(3-dimethylaminopropyl-)carbodiimide and N-hydroxysulfosuccinimide (Pierce) were incubated with Hyal-linked Sepharose beads for 5 h under different pH conditions (pH 1-9). The amount of biotinylated HA released from the beads was measured by alkaline phosphatase-conjugated avidin in the presence of p-nitrophenyl phosphate and recorded by a Molecular Devices (Spectra Max 250) ELISA reader at a wavelength of 405 nm as previously described (Bourguignon et al., 2004).

HABP2 protein purification and protease activity determination. Media from HABP2 overexpression vector-transfected EC (Origene) was immunoprecipitated with anti-HABP2 antibody-conjugated Sepharose beads (Sigma). The immunobeads were then extensively washed in 50 mM Borate, pH=8.5 and eluted with 1M NaCl with 0.1% NP-40 and dialyzed against a 1,000 fold excess of 50 mM Borate, pH=8.5. Protein assays were performed to quantitate total protein. The purified protein was run on SDS-PAGE and either immunoblotted with anti-HABP2 antibody (Novus Biologicals) or stained with Imperial™ protein stain (Pierce) to check for purity. Protease activity was measured using the QuantiCleave™ Protease Assay Kit (Pierce). Briefly, the immunobeads were incubated with succinylated casein for one hour followed by development with TNBSA (2,4,6-trinitrobenzene sulfonic acid) and read at 450 nm.

Statistical analysis. Student's t test was used to compare the means of data from two or more different experimental groups. Results were expressed as means±S.E.

B. Results

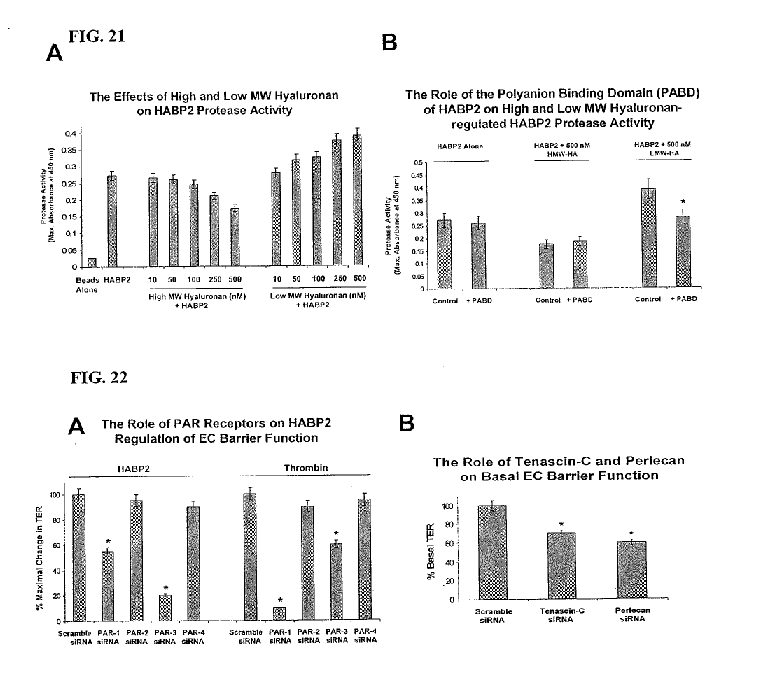

Analysis of HABP2 expression and hyaluronan regulation of purified HABP2 activity. Hyaluronic Acid Binding Protease 2 (HABP2) was an extracellular serine protease highly expressed in lungs. HABP2 contains 3 EGF-like domains, a kringle-like domain and a trypsin-like protease domain. The polyanion binding domain (PABD) was contained within the second and third EGF-like domains. HABP2 expression in human pulmonary endothelial cells (EC) was suggested by immunoblotting. Further, the EC barrier disrupting agents lipopolysaccharide (LPS) and low molecular weight hyaluronan (LMW-HA) increased HABP2 expression while the EC barrier enhancing agent, high molecular weight hyaluronan (HMW-HA) decreased HABP2 expression in human EC. Using purified HABP2 isolated from HABP2 overexpressing EC indicates that HMW-HA inhibits, while LMW-HA activates, HABP2 protease activity (FIG. 21A). The effects of LMW-HA, but not HMW-HA, were blocked with a recombinant peptide of the PABD of HABP2 (FIG. 21B).

The role of HABP2 in pulmonary EC barrier function. Purified HABP2 induces a rapid transient decrease in EC barrier function which is similar to another serine protease, thrombin. Silencing HABP2 expression (siRNA) augmented HMW-HA-mediated EC barrier enhancement while inhibiting LMW-HA and LPS-induced EC barrier disruption. These effects were reversed with HABP2 overexpression (FIG. 17). To understand the mechanism of HABP2-mediated EC barrier disruption, protease activated receptors (PAR) and extracellular matrix (ECM) components were examined. FIG. 22A indicates that silencing (siRNA) PAR-1 or PAR-3 (but not PAR-2 or PAR-4) receptor inhibits both HABP2 and thrombin-mediated EC barrier disruption. Overexpression of HABP2 in human EC selectively degraded the ECM components, tenascin-C and perlecan by immunoblotting experiments. Silencing (siRNA) tenascin-C or perlecan expression decreases basal EC barrier function (FIG. 22B).

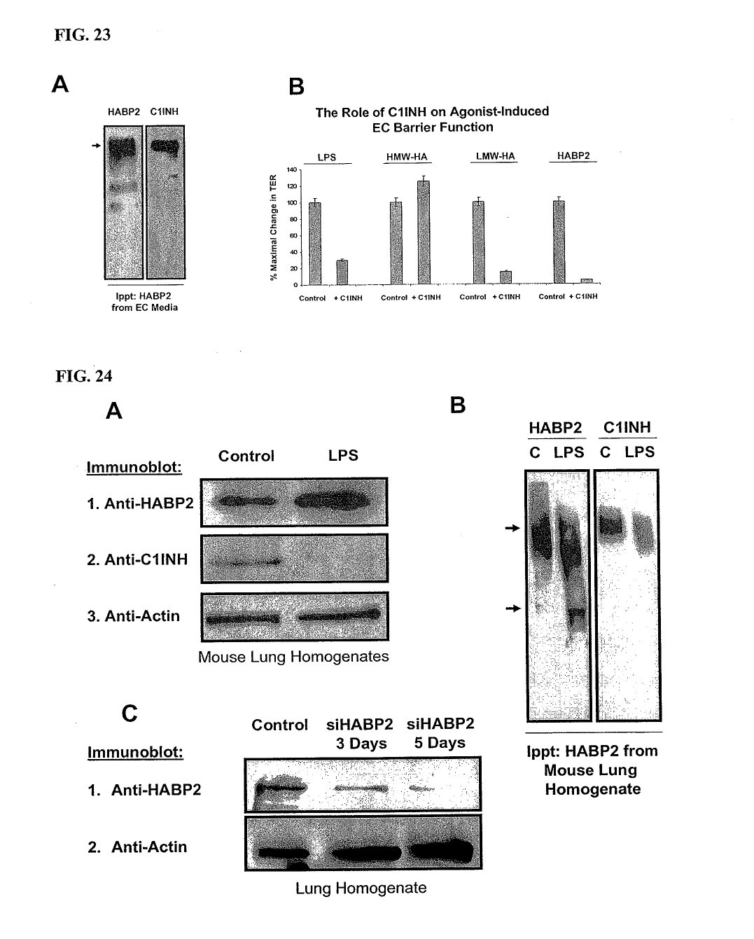

The role of the extracellular serine protease inhibitor, C1INH, in HABP2-regulated pulmonary EC barrier regulation. C1INH was expressed in human EC determined by immunublotting. Further, the EC barrier disrupting agents lipopolysaccharide (LPS) and low molecular weight hyaluronan (LMW-HA) decreased HABP2 expression while the EC barrier enhancing agent, high molecular weight hyaluronan (HMW-HA) increased HABP2 expression in human EC. FIG. 23A indicates that extracellular C1INH forms an SDS-stable complex with HABP2 in media from cultured human EC. Further, purified C1INH inhibited the EC barrier disrupting effects of LPS, LMW-HA and HABP2 while enhancing the barrier protective effects of HMW-HA (FIG. 23B).

The roles of HABP2 and C1INH in LPS-induced acute lung injury (ALI) in vivo. FIG. 24A indicates that both HABP2 and C1INH were expressed in the mouse lung. Intratracheal LPS challenge (24 hours) increased HABP2 and decreased C1INH protein expression. Further, C1INH formed an SDS-stable complex with HABP2 in vivo which was inhibited with LPS challenge, allowing for the free (active) form of HABP2 to be expressed (FIG. 24B). Finally, the expression of HABP2 was successfully silenced using intravenous administration of a stable form of siRNA (siSTABLE, Dharmacon) against murine HABP2 (FIG. 24C).

### Example 7

### Hyaluronan (HA) Regulation of Tumor-Associated Angiogenesis

A. Results

Determination of the role of HMW-HA/CD44 interactions in regulating hyaluronidase activity and angiogenesis in vitro. Human pulmonary microvascular EC (HPMVEC) (Cambrex), grown in EBM-2 complete medium (Cambrex) at 37° C. in a humidified atmosphere of 5% CO₂, 95% air, with passages 6-10 used for experimentation was treated with or without 100 nM HMW-HA and/or 100 nM VEGF in the presence or absence of CD44 isoform-specific blocking antibodies (EMD Biosciences), siRNA (scramble, CD44, Hyal-1, Hyal-2, Hyal-3 or Hyal-4) and the potent hyaluronidase inhibitor, L-ascorbic acid 6-hexadecanoate (Botzki et al., 2004) (Sigma). Target sequences for siRNA were generated by scanning the target gene and identifying unique 19 nucleotide unique sequences. Sense and antisense DNA 29 oligonucleotide (21 nucleotides encoding the siRNA, 8 nucleotides encoding a T7 promoter primer, were generated against identified target sequences. Then, the double stranded RNA were made and transfected into human pulmonary EC at a concentration of 10 nM using Ambion siRNA transfection reagent. A scramble sequence that does not have any known gene target was transfected as a control. Verification of siRNA efficiency was determined using immunoblotting with a specific antibody. Immunoblotting with antibodies against non-target proteins (including actin) was used to determine the specificity of the siRNA. Treated EC will either be analyzed for protein expression, hyaluronidase activity or VEGF-induced proliferation, migration or tube formation. Cellular lysates were obtained with lysis buffer (50 mM HEPES (pH 7.5), 150 mM NaCl, 20 mM MgCl₂, 1% Triton X-100, 0.1% SDS, 0.4 mM Na₃VO₄, 40 mM NaF, 50 μM okadaic acid, 0.2 mM phenylmethylsulfonyl fluoride, 1:250 dilution of Calbiochem protease inhibitor mixture 3). The samples were then run on SDS-PAGE in 4-15% polyacrylamide gels, transfer onto Immobilon™ membranes, and developed with specific primary (i.e. anti-CD44, Hyal-1, Hyal-2, Hyal-3 or Hyal-4 antibody) and secondary antibodies. Visualization of immunoreactive bands were achieved using enhanced chemiluminescence (Amersham Biosciences) as described previously (Singleton et al., 2005, 2006). For EC migration, twenty-four transwell units with 8 μM pore size were used for monitoring in vitro cell migration. HPMVEC (˜1×10⁴ cells/well) were plated with various treatments (see above) to the upper chamber and VEGF, HMW-HA and/or LMW-HA (100 nM) were added to the lower chamber. Cells were allowed to migrate for 18 hours. Cells from the upper and lower chamber were quantitated using the CellTiter96™ MTS assay (Promega, San Luis Obispo, Calif.) and read at 492 nm. % migration was defined as the # of cells in the lower chamber % the number of cells in both the upper and lower chamber. Each assay was set up in triplicate, repeated at least five times and analyzed statistically by Student's t test (with statistical significance set at P<0.05). For EC proliferation, HPMVEC [5×10³ cells/well pretreated with various agents (see above) were incubated with 0.2 ml of serum-free media containing various agonists (100 nM MMW-HA, LMW-HA or VEGF) for 24 h at 37° C. in 5% CO₂/95% air in 96-well culture plates. The in vitro cell proliferation assay was analyzed by measuring increases in cell number using the CellTiter96™ MTS assay (Promega, San Luis Obispo, Calif.) and read at 492 nm. Each assay was set up in triplicate, repeated at least five times and analyzed statistically by Student's t test (with statistical significance set at P<0.05) as we have previously described. For tube formation, glass coverslips were coated with a thin layer of Matrigel (0.250 mL) with or without VEGF (100 nM) which was allowed to gel for 30 min at 37° C. before use. When the matrix has solidified, treated EC (see above) were seeded in multiple 35 mm dishes at a density of ˜1.5-2×10⁵ cells per dish. After plating, cells were incubated in 5% CO₂ at 37° C. before fixation and processing for immunofluorescence. The fixed EC were examined with a Nikon TE200 inverted microscope equipped for epifluorescence and digitally imaged with a Spot Camera (Diagnostics Instruments).

Determination of the role of HMW-HA/HABP2/C1INH interactions in regulating angiogenesis in vitro. Human pulmonary EC (Cambrex), grown in EBM-2 complete medium (Cambrex) at 37° C. in a humidified atmosphere of 5% CO₂, 95% air, with passages 6-10 used for experimentation will be treated with or without 100 nM HMW-HA and/or 100 nM VEGF in the presence or absence of HABP2 and/or C1INH overexpression vectors (Origene), exogenous purified recombinant HABP2 polyanion binding domain and/or C1INH (Novus Biologicals) and/or siRNA (scramble, HABP2 or C1INH). For HABP2 protease activity determination, media from HABP2 overexpressing EC (see above) was immunoprecipitated with anti-HABP2 antibody-conjugated Sepharose beads (Sigma). The immunobeads were then extensively washed in 50 mM Borate, pH=8.5 and protease activity was measured using the QuantiCleave™ Protease Assay Kit (Pierce). Briefly, the immunobeads were incubated with succinylated casein for one hour followed by development with TNBSA (2,4,6-trinitrobenzene sulfonic acid) and read at 450 nm.

Statistical analysis. Student's t test was used to compare the means of data from two or more different experimental groups. Results were expressed as means±S.E.

B. Results

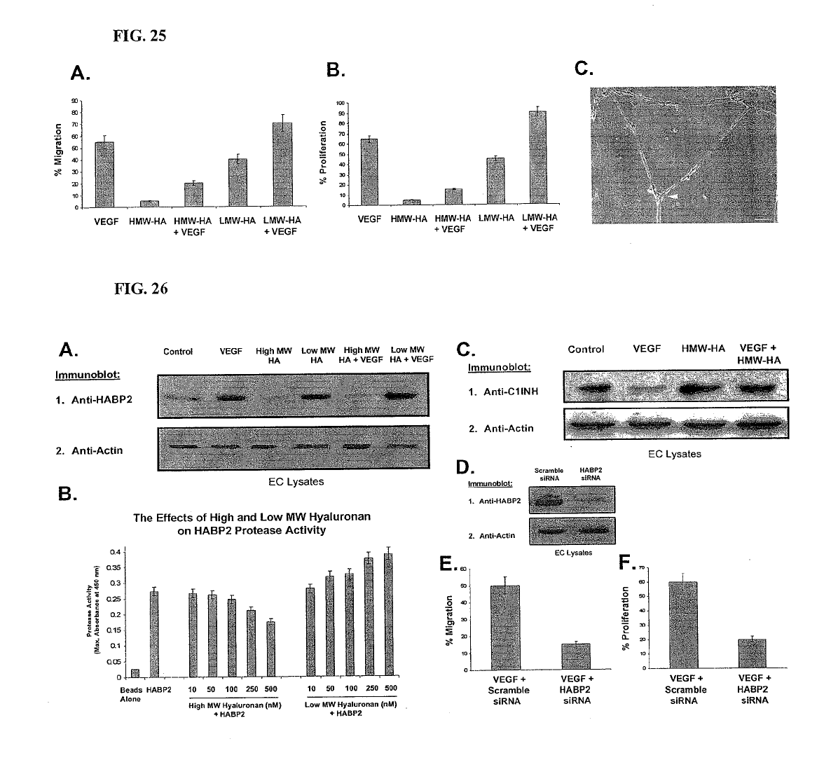

Analysis of CD44 isoform and hyaluronidase expression and HA effects on VEGF-induced angiogenic events. FIG. 25 indicates that HMW-HA inhibits VEGF-induced angiogenic events and hyaluronidase expression while LMW-HA promotes EC proliferation and migration. Human pulmonary microvascular EC expressed CD44 isoforms CD44s and CD44v10. HMW-HA inhibited VEGF-induced hyaluronidase expression, EC proliferation (A) and migration (B). Further, EC tube formation was successfully induced (C).

Analysis of HABP2 and C1INH expression, HA regulation of HABP2 activity and HABP2 regulation of VEGF-induced angiogenic events. FIG. 26 indicates that HMW-HA inhibits, while LMW-HA enhances, HABP2 expression (A) and activity (B) in human EC. Further, HMW-HA increased the expression of the endogenous inhibitor of HABP2, C1INH(C). Silencing HABP2 (siRNA) (D) inhibited VEGF-induced angiogenic events (E, F).

EC functions in vivo. Analysis of bronchioalveolar lavage (BAL) fluid proteins in wildtype or CD44 knockout mice showed that HWM-HA protected from LPS-induced vascular leakiness in vivo and this HMW-HA protection was blocked in the CD44 knockout mouse (refer to FIG. 18A).

### Example 8

### Silencing HABP2 Expression in Mice Protects from LPS-Induced Acute Lung Injury (ALI)

The effect on HABP2 silencing was evaluated in mice using HAPB2 siRNA molecules. The level of HABP2 in pulmonary endothelial cells was evaluated in LPS-treated mice. Immunohistochemical fluorescent staining images of control or LPS-treated (2.5 mg/kg, intratracheal, 24 hours=acute lung injury (ALI), right panels) mouse lungs was conducted using either bright field (DIC) imaging, DAPI staining (which stains nuclei), treatment with anti-HABP2 antibody, treatment with anti-Factor VIII (vWB) antibody (which labels endothelial cells) and secondary fluorescent antibody (Alexa Fluor™ 610 (for HABP2) and 350 (for vWB), Molecular Probes). Based on staining patterns, increased HABP2 expression was observed and this was associated with pulmonary endothelial cells with LPS treatment.

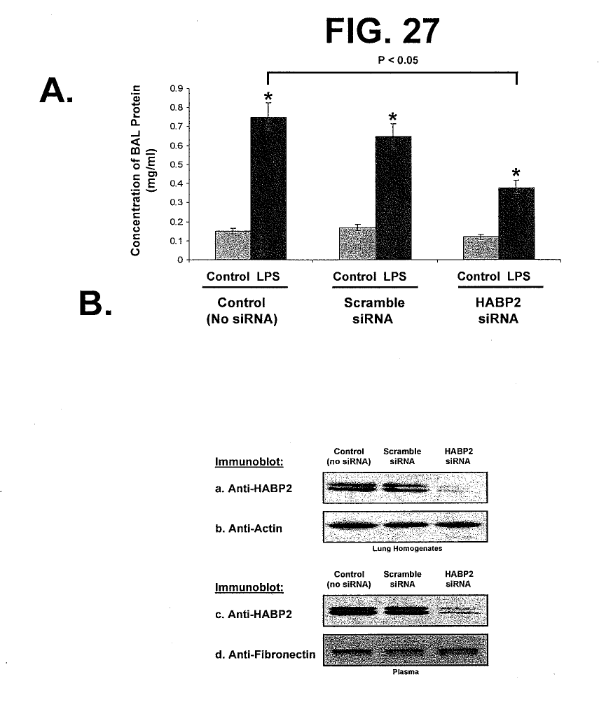

Male C57BL/6J mice were anesthetized and were given either saline (control), scramble siRNA (which does not target any known murine mRNA) or siSTABLE HABP2 siRNA (Dharmacon) intravenously. After 4 days, the mice were either given saline (control) or LPS (2.5 mg/kg) intratracheally. The treated mice were allowed to recover for 24 hours, bronchioalveolar lavage (BAL) fluids were obtained and analyzed for protein concentrations (FIG. 27B) and plasma was obtained and lungs were extracted and homogenized for immunoblot analysis (FIG. 27A). For Panel A, homogenized lungs (a,b) and plasma (c,d) were run on SDS-PAGE, transferred to nitrocellulose membranes and probed with either anti-HABP2 (a,c), anti-actin (b) or anti-fibronectin (d) antibodies followed by specific secondary antibodies. The results indicated successful inhibition of HABP2 protein expression with HABP2 siSTABLE siRNA in mouse lung and serum. For Panel B, the y-axis indicates the concentration of BAL protein (mg/ml) for each pooled N=5 sample. The single asterisk (*) refers to a significant (p<0.05) difference between control and LPS treatment. There was also a significant difference (p<0.05) between control (no siRNA)+LPS and HABP2 siSTABLE siRNA+LPS treatment indicating silencing HABP2 protein expression protected mice from LPS-induced ALI.

### REFERENCES

The following references, to the extent that they provide exemplary procedural or other details supplementary to those set forth herein, are specifically incorporated herein by reference.
U.S. Pat. No. 4,415,723
U.S. Pat. No. 4,458,066
U.S. Pat. No. 4,816,567
U.S. Pat. No. 4,870,287
U.S. Pat. No. 5,633,003
U.S. Pat. No. 5,739,169
U.S. Pat. No. 5,760,395
U.S. Pat. No. 5,795,715
U.S. Pat. No. 5,801,005
U.S. Pat. No. 5,824,311
U.S. Pat. No. 5,830,880
U.S. Pat. No. 5,846,945
U.S. Pat. No. 5,889,136
U.S. Pat. No. 6,651,655
U.S. Patent Publn. 2002/0086852
Aderem, Biochem. Soc. Trans., 23:587-591, 1995.
Altincicek et al., Biochem. J., 394: 687-692, 2006.
Angiolillo et al., J. Exp. Med., 182(1):155-162, 1995.
Arnold and West, Pharmacol. Ther., 52:407-422, 1991.
Austin-Ward and Villaseca, Revista Medica de Chile, 126(7):838-845, 1998.
Bajorin et al., J. Clin. Oncol., 6(5):786-792, 1988.
Bensadoun et al., Am. J. Respir. Crit. Care Med., 154:1819-1828, 1996.
Blomback, Thromb Res., 75:327-328, 1994.
Bosher and Labouesse, Nat. Cell. Biol., 2(2):E31-E36, 2000.
Botzki et al., J. Biol. Chem., 279:45990-45997, 2004.
Bourguignon et al., Front Biosci., 3:d637-649, 1998.
Bourguignon et al., J. Biol. Chem., 279:26991-27007, 2004.
Bourguignon et al., J. Biol. Chem., 279:29654-29669, 2004.
Brower, Crit. Care Clin., 18(1):1-13, 2002.
Brummelkamp et al., Cancer Cell, 2:243-247, 2002.
Brummelkamp et al., Science, 296(5567):550-553, 2002.
Bukowski et al., Clinical Cancer Res., 4(10):2337-2347, 1998.
Calabro et al., Glycobiology, 10:283-293, 2000.
Cantor and Turino, Chest., 125:288-292, 2004.
Cao et al., J. Exp. Med., 182(6):2069-2077, 1995.
Caplen et al., Gene, 252(1-2):95-105, 2000.
Cardones and Banez, Curr. Pharm. Des., 12:387-394, 2006.
Cetin et al., Surgery, 136:375-383, 2004.
Chen et al., Am. J. Hum. Genet., 57(1):133-149, 1995.
Christodoulides et al., Microbiology, 144(Pt 11):3027-3037, 1998.
Clackson et al., Nature 352: 624-628, 1991.
Clapp, Clin. Perinatol., 20(1):155-168, 1993.
Davidson et al., J. Immunother., 21(5):389-398, 1998.
Dempfle, Thromb. Haemost., 82:673-683, 1999.
Dentener et al., Thorax., 60:114-119, 2005.
Dhanabal et al., Curr. Med. Chem. Anticancer. Agents, 5:115-130, 2005.
Dillman, Cancer Biother. Radiopharm., 14(1):5-10, 1999.
Drab et al., Science, 293:2449-2452, 2001.
Dudek and Garcia, J. Appl. Physiol., 91:1487-1500, 2001.
Dudek et al., J. Biol. Chem., 279:24692-24700, 2004.
Dvorak, Methods, 29:86-96, 2003.
Elbashir et al., Genes Dev., 5(2):188-200, 2001.
Elbashir et al., Nature, 411(6836):494-498, 2001.
Erlandsson, Cancer Genet. Cytogenet., 104(1):1-18, 1998.
Felmeden and Lip, Expert Opin. Investig. Drugs, 14:1319-1336, 2005.
Finigan et al., J. Biol. Chem., 280:17286-17293, 2005.
Fire et al., Nature, 391(6669):806-811, 1998.
Folkman et al., Ann. Surg., 214:414-425, 1991.
Folkman et al., Nature, 339(6219):58-61, 1989.
Folkman, Nat. Med., 1:27-31, 1995.
Garcia et al., J. Clin. Invest., 108:689-701, 2001.
Garrean et al., J. Immunol., 177:4853-4860, 2006.
Gaya and Rustin, Clin. Oncol. (R. Coll. Radiol.), 17:277-290, 2005.
Gee et al., Arch. Immunol. Ther. Exp. (Warsz), 52:13-26, 2004.
Glade-Bender et al., Expert Opin. Biol. Ther., 3:263-276, 2003.
Good et al., Proc. Natl. Acad. Sci. USA, 87(17):6624-6628, 1990.
Grishok et al., Science, 287:2494-2497, 2000.
Gupta et al., Proc. Natl. Acad. Sci. USA, 92(17):7799-7803, 1995.
Hammond et al., Curr. Top. Microbiol. Immunol., 286:21-44, 2004.
Hanahan, Science, 277:48-50, 1997.
Hanibuchi et al., Int. J. Cancer, 78(4):480-485, 1998.
Harder and Simons, Curr. Opin. Cell Biol., 9:534-542, 1997.
Hasleton et al., Histopathology, 34(4):285-294, 1999.
Hellstrand et al., Acta Oncologica, 37(4):347-353, 1998.
Henderson et al., News Physiol. Sci., 19:39-43, 2004.
Hirano et al., Int. Immunol., 6:49-59, 1994.
Hui and Hashimoto, Infection Immun., 66(11):5329-5336, 1998.
Ikenoya et al., J. Neurochem., 81:9-16, 2002.
Ilangumaran et al., Blood, 91:3901-3908, 1998.
Ilangumaran et al., Leuk. Lymphoma., 35:455-469, 1999.
Irie and Morton, Proc. Natl. Acad. Sci. USA, 83(22):8694-8698, 1986.
Irie et al., Lancet., 1(8641):786-787, 1989.
Irie et al., Science, 277:1511-1515, 1997.
Ishizawa et al., Biochem. Biophys. Res. Commun., 324:276-280, 2004.
Ju et al., Gene Ther., 7(19):1672-1679, 2000.
Kandel et al., Cell, 66(6):1095-1104, 1991.
Kannemeier et al., Eur. J. Biochem., 268:3789-3796, 2001.
Kermorgant and Parker, Cell Cycle, 4:352-355, 2005.
Ketting et al., Cell, 99(2):133-141, 1999.
Kim et al., Nature, 362(6423):841-844, 1993.
Kohler and Milstein, Nature, 256:495-497, 1975.
Kolmel, J. Neurooncol., 38(2-3):121-125, 1998.
Legg et al., Nat. Cell. Biol., 4:399-407, 2002.
Lin and Avery, Nature, 402:128-129, 1999.
Liu et al., Faseb J., 16:950-962, 2002.
Lokeshwar et al., J. Biol. Chem., 271:23853-23864, 1996.
Lucius et al., J. Membr. Biol., 194:97-108, 2003.
Luscher and Barton, Clin. Cardiol., 20:II-3-10, 1997.
Ma et al., Cancer Metastasis Rev., 22:309-325, 2003.
Magi-Galluzzi et al., Anal. Quant. Cytol. Histol., 20(5):343-350, 1998.
Maione et al., Science, 247(4938):77-79, 1990.
Mangray and King, Front Biosci., 3:D1148-1160, 1998.
Marks et al., J. Mol. Biol. 222: 581-597, 1991.
Mathew et al., Circulation, 110:1499-1506, 2004.
Matsubara, Seikagaku, 77:50-55, 2005.
Miklaszewska et al., Przegl Lek., 61:192-198, 2004.
Milhiet et al., J. Soc. Biol., 198:169-174, 2004.
Millauer et al., Nature, 367(6463):576-579, 1994.
Min and Cowman, Anal. Biochem., 155:275-285, 1986.
Minshall et al., Am. J. Physiol. Lung Cell Mol. Physiol., 285:L1179-1183, 2003.
Mitchell et al., Ann. NY Acad. Sci., 690:153-166, 1993.
Mitchell et al., J. Clin. Oncol., 8(5):856-869, 1990.
Mohamadzadeh et al., J. Clin. Invest., 101:97-108, 1998.
Montgomery et al., Proc. Natl. Acad. Sci. USA, 95:15502-15507, 1998.
Morton et al., Arch. Surg., 127:392-399, 1992.
Mougin et al., Ann. Biol. Clin., (Paris) 56(1): 21-8, 1998.
Mumby and Walter, Cell Regul., 2(8):589-98, 1991.
Mustafa et al., J. Immunother., 25:476-488, 2002.
Natoli et al., Biochem. Pharmacol., 56(8):915-20, 1998.
Nettelbladt and Hallgren, Am. Rev. Respir. Dis., 140:1028-1032, 1989.
Noma et al., Am. J. Physiol. Cell Physiol., 290:C661-668, 2006.
O'Reilly et al., Cell, 79(2):315-328, 1994.
Orian-Rousseau et al., Genes Dev., 16:3074-3086, 2002.
Orian-Rousseau et al., Mol. Biol. Cell, 18:76-83, 2007.
Parangi et al., Proc. Natl. Acad. Sci. USA, 93(5):2002-2007, 1996.
Parat et al., Mol. Biol. Cell, 14:3156-3168, 2003.
PCT Appln. WO 00/44914
PCT Appln. WO 01/68836
PCT Appln. WO 01/93846
PCT Appln. WO 99/32619
Pearson, Radiology, 179:9-14, 1991.
Peng et al., Am. J. Respir. Crit. Care Med., 169:1245-1251, 2004.
Pietras et al., Oncogene, 17(17):2235-2249, 1998.
Pogrel et al., J. Oral Sci., 45:85-91, 2003.
Qin et al., Proc. Natl. Acad. Sci. USA, 95(24):14411-14416, 1998.
Rafi-Janajreh et al., J. Immunol., 163:1619-1627, 1999.
Rastinejad et al., Cell, 56(3):345-355, 1989.
Ravindranath and Morton, Intern. Rev. Immunol., 7: 303-329, 1991.
Reich et al., Biotechnol. Adv., 19:451-485, 2001.
Remington's Pharmaceutical Sciences, 18th Ed. Mack Printing Company, 1289-1329, 1990.
Ren et al., Embo J., 18:578-585, 1999.
Risau, Nature, 386:671-674, 1997.
Romisch, Biol. Chem., 383:1119-1124, 2002.
Rosenberg et al., Ann. Surg. 210(4):474-548, 1989.
Rosenberg et al., N. Engl. J. Med., 319:1676, 1988.
Scott and Heatley, Biomacromolecules, 3:547-553, 2002.
Seyfried et al., Glycobiology, 15:303-312, 2005.
Sharp, Genes Dev., 13:139-141, 1999.
Shifren et al., Proc. Am. Thorac. Soc., 3(5):428-433, 2006.
Shigemura et al., Circulation, 111:1407-1414, 2005.
Singleton and Bourguignon, Cell. Motil. Cytoskeleton, 53:293-316, 2002.
Singleton and Bourguignon, Exp. Cell Res., 295:102-118, 2004.
Singleton et al., Faseb J., 19:1646-1656, 2005.
Singleton et al., J. Biol. Chem., 281:34381-34393, 2006.
Singleton et al., Microvasc. Res., 72:3-11, 2006.
Sivan et al., Pediatr. Pulmonol., 8(2):104-108, 1990.
Slevin et al., J. Biol. Chem., 277:41046-41059, 2002.
Solyanik et al., Cell. Prolif., 28(5):263-278, 1995.
Stokke et al., Cell. Prolif., 30(5):197-218, 1997.
Strieter et al., J. Biol. Chem., 270(45):27348-27357, 1995.
Su et al., Intensive Care Med., 30:133-140, 2004.
Sui et al., Proc. Natl. Acad. Sci. USA, 99(8):5515-5520, 2002.
Sundaram et al., Biochem. Cell. Biol., 82:191-200, 2004.
Tabara et al., Cell, 99(2):123-132, 1999.
Taher et al., Curr. Top Microbiol. Immunol., 246:31-37, 1999.
Tanabe et al., Biochem. Biophys. Res. Commun., 345:156-161, 2006.
Tasaka et al., Am. J. Respir. Cell Mol. Biol., 32:504-510, 2005.
Teder and Heldin, Am. J. Respir. Cell. Mol. Biol., 17:376-385, 1997.
Teder et al., Science, 296:155-158, 2002.
Toole, Nat. Rev. Cancer, 4:528-539, 2004.
Turley et al., J. Biol. Chem., 277:4589-4592, 2002.
Tzircotis et al., Oncogene, 25:7401-7410, 2006.
van der Voort et al., J. Biol. Chem., 274:6499-6506, 1999.
Voest et al., J. Natl. Cancer Inst., 87(8):581-586, 1995.
Volonte et al., FEBS Lett., 445:431-439, 1999.
Ware et al., Keio J. Med., 54:142-149, 2005.
Weiss et al., J. Invest. Dermatol., 111:227-232, 1998.
Wincott et al., Nucleic Acids Res., 23(14):2677-2684, 1995.
Wygrecka et al., J. Biol. Chem., 282:21671-21682, 2007.
Wygrecka et al., Thorax, 62(10):880-888, 2007.
Zbikowska et al., Blood Coagul. Fibrinolysis, 18:271-277, 2007.
Zhao and Davis, J. Cell Biochem., 79:496-505, 2000.

1. A method for regulating vascular permeability in a subject having symptoms of or diagnosed with a disease or condition involving increased vascular permeability comprising administering to the subject an effective amount of a composition comprising substantially purified hyaluronan, wherein the hyaluronan is at least about 95% pure hyaluronan with a molecular weight greater than about 500 kilodaltons.

2. The method of claim 1, wherein the hyaluronan has a molecular weight of at least about one million daltons.

3. The method of claim 1, wherein the composition does not contain a detectable amount of hyaluronan with a molecular weight below about 250 kilodaltons.

4. The method of claim 3, wherein the composition does not contain detectable amounts of hyaluronan with a molecular weight below about 500 kilodaltons.

5. The method of claim 1, wherein the composition is 95% pure hyaluronan with respect to other biological macromolecules.

6.-8. (canceled)

9. The method of claim 1, wherein the composition does not contain detectable amounts of nucleic acids, chondroitin sulfate, and/or any endotoxins.

10. The method of claim 1, wherein the hyaluronan was purified using a size exclusion filter or gel filtration chromatography.

11. (canceled)

12. The method of claim 1, further comprising identifying a subject with symptoms of or a diagnosis of a disease or condition involving increased vascular permeability.

13. The method of claim 1, wherein the subject is a human.

14. The method of claim 1, wherein the disease or condition involving increased vascular permeability is acute lung injury (ALI), acute respiratory distress syndrome (ARDS), atherosclerosis, macular degeneration, capillary leakage syndrome, or sepsis.

15. The method of claim 14, wherein the disease or condition is ALI or ARDS and the composition reduces or eliminates one or more symptoms of ALI or ARDS.

16. The method of claim 15, wherein the subject has or will be treated with one or more corticosteroids or mechanical ventilation.

17. The method of claim 1, wherein the composition is administered to the subject by inhalation.

18. The method of claim 17, wherein the composition is administered to the subject as an aerosol.

19. The method of claim 1, wherein the composition is administered to the subject intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, via nebulizer, or via a lavage.

20. The method of claim 1, wherein the composition is administered multiple times.

21. The method of claim 1, wherein the about 1 μg hyaluronan/kg body weight to about 50 mg hyaluronan/kg body weight is administered to the subject.

22. (canceled)

23. A method for regulating vascular permeability in a subject having a disease or condition involving increased vascular permeability comprising administering to the subject an effective amount of a composition comprising purified hyaluronan, wherein more than 95% of the hyaluronan in the composition has a molecular weight of at least about 500 kilodaltons.

24. A method for treating a lung disease or condition involving endothelial cells in the lungs comprising administering to a patient in need of such treatment an effective amount of a composition comprising purified hyaluronan, wherein the hyaluronan has a molecular weight above about 500 kilodaltons.

25. The method of claim 24, wherein the lung disease or condition is acute lung injury (ALI) or acute respiratory distress syndrome (ARDS).

26.-73. (canceled)


SEQUENCE LISTING (38 sequences)

SEQ ID NO: 1 - DNA, 3019 bp, Homo sapiens
  CDS (104)..(1786)
atttgttcctgaatccttggagactgacatttttcccccctaaaggcatagacaacaaaagaaattttattgagaggaaaacacaagtccttaaactgcaaagatgtttgccaggatgtctgatctccatgttctgctgttaatggctctggtgggaaagacagcctgtgggttctccctgatgtctttattggaaagcctggacccagactggacccctgaccagtatgattacagctacgaggattataatcaggaagagaacaccagtagcacacttacccacgctgagaatcctgactggtactacactgaggaccaagctgatccatgccagcccaacccctgtgaacacggtggggactgcctcgtccatgggagcaccttcacatgcagctgcctggctcctttctctgggaataagtgtcagaaagtgcaaaatacgtgcaaggacaacccatgtggccggggccaatgtctcattacccagagtcctccctactaccgctgtgtctgtaaacacccttacacaggtcccagctgctcccaagtggttcctgtatgcaggccaaacccctgccagaatggggctacctgctcccggcataagcggagatccaagttcacctgtgcctgtcccgaccagttcaaggggaaattctgtgaaataggttctgatgactgctatgttggcgatggctactcttaccgagggaaaatgaataggacagtcaaccagcatgcgtgcctttactggaactcccacctcctcttgcaggagaattacaacatgtttatggaggatgctgaaacccatgggattggggaacacaatttctgcagaaacccagatgcggacgaaaagccctggtgctttattaaagttaccaatgacaaggtgaaatgggaatactgtgatgtctcagcctgctcagcccaggacgttgcctacccagaggaaagccccactgagccatcaaccaagcttccggggtttgactcctgtggaaagactgagatagcagagaggaagatcaagagaatctatggaggctttaagagcacggcgggcaagcacccatggcaggcgtccctccagtcctcgctgcctctgaccatctccatgccccagggccacttctgtggtggggcgctgatccacccctgctgggtgctcactgctgcccactgcaccgacataaaaaccagacatctaaaggtggtgctaggggaccaggacctgaagaaagaagaatttcatgagcagagctttagggtggagaagatattcaagtacagccactacaatgaaagagatgagattccccacaatgatattgcattgctcaagttaaagccagtggatggtcactgtgctctagaatccaaatacgtgaagactgtgtgcttgcctgatgggtcctttccctctgggagtgagtgccacatctctggctggggtgttacagaaacaggaaaagggtcccgccagctcctggatgccaaagtcaagctgattgccaacactttgtgcaactcccgccaactctatgaccacatgattgatgacagtatgatctgtgcaggaaatcttcagaaacctgggcaagacacctgccagggtgactctggaggccccctgacctgtgagaaggacggcacctactacgtctatgggatagtgagctggggcctggagtgtgggaagaggccaggggtctacacccaagttaccaaattcctgaattggatcaaagccaccatcaaaagtgaaagtggcttctaaggtactgtcttctggacctcagagcccactctccttggcaccctgacaccgggaggcctcatggccaacaatggacacctccagagcctccaggggaccacacagtagactatccctactctaagcagagacaactgccacccagcctgggccttcccagaccagcatttgcacaatatcaccaggcttcttctgcctcccttggtaacccaaggaatgatggaatcaacacaacatagtatgtttgctttccttacccaattgtaccttctagaaaatcagtgttcacagagactgcctccaccacaggcatcctgcaaatgcagactccagaatccccagcatcagcgggaaccaccatcacatctttattcctcagcccagacactcgaggcactcaacagaatcagccatccacgtctaggtatcagagaggaccacaaatacaacattctccatctgctttcagagttattattttaataaaggaagatctgggatgggctggtgggccattccagcttgccgaaatcaaagccatctgaagcctgtctctggtgaacaaacttcctctctggcctctcaggaatcagggtggacatggctcacaacagcagggccttcttctttttgacgtgcagaatctcagtggcatctgggttcacctccccactctgatgatctccagcctccactgcttctgccccccgctgctgaaatcaaacataccccaagttaaaatgaagctcccccacccccactcccggccccggttcccacaggacacgctaagaagcacagggagcatttaacaggctcaccctccctttccttttcccctcttctaccctccccaagaaaaagggccttcaaggcaggaatgagaaagcaaagccaatctctcatttagacctggcttctttcttctgaacaaagtagggttcaaaatgcagactgtcatatccagcgagtccctgaccctttctgcgaatgtaacgagcaagcagtcagcacagcctgggctgccctggcccgggattgatgtagccccggtaggtttgcctctgcagaactaatggctgtgacttcagagaaagccctgcaggaagtttaacctgcgtgtcatctgcctggtcatctcagacccatgaaattaggcgccttgtttgagctgcgtttcacacttctttagagctagctgacctttggccaaaaataaactttgaaaagaaa

SEQ ID NO: 2 - protein, 560 aa, Homo sapiens
MFARMSDLHVLLLMALVGKTACGFSLMSLLESLDPDWTPDQYDYSYEDYNQEENTSSTLTHAENPDWYYTEDQADPCQPNPCEHGGDCLVHGSTFTCSCLAPFSGNKCQKVQNTCKDNPCGRGQCLITQSPPYYRCVCKHPYTGPSCSQVVPVCRPNPCQNGATCSRHKRRSKFTCACPDQFKGKFCEIGSDDCYVGDGYSYRGKMNRTVNQHACLYWNSHLLLQENYNMFMEDAETHGIGEHNFCRNPDADEKPWCFIKVTNDKVKWEYCDVSACSAQDVAYPEESPTEPSTKLPGFDSCGKTEIAERKIKRIYGGFKSTAGKHPWQASLQSSLPLTISMPQGHFCGGALIHPCWVLTAAHCTDIKTRHLKVVLGDQDLKKEEFHEQSFRVEKIFKYSHYNERDEIPHNDIALLKLKPVDGHCALESKYVKTVCLPDGSFPSGSECHISGWGVTETGKGSRQLLDAKVKLIANTLCNSRQLYDHMIDDSMICAGNLQKPGQDTCQGDSGGPLTCEKDGTYYVYGIVSWGLECGKRPGVYTQVTKFLNWIKATIKSESGF

SEQ ID NO: 3 - DNA, 22 bp, Artificial
  Synthetic primer
ccaagatgatcagccattctgg

SEQ ID NO: 4 - DNA, 20 bp, Artificial
  Synthetic primer
aagacatctaccccagcaac

SEQ ID NO: 5 - DNA, 20 bp, Artificial
  Synthetic primer
ggtggaagaagagacccaaa

SEQ ID NO: 6 - DNA, 21 bp, Artificial
  Synthetic primer
aagctacacaaaaagcctgga

SEQ ID NO: 7 - DNA, 21 bp, Artificial
  Synthetic primer
aaaaagcctggatcactcatc

SEQ ID NO: 8 - DNA, 21 bp, Artificial
  Synthetic primer
aacagggactcagggaccaga

SEQ ID NO: 9 - DNA, 21 bp, Artificial
  Synthetic primer
aaatgaatgttcctggggcgc

SEQ ID NO: 10 - DNA, 21 bp, Artificial
  Synthetic primer
aatataacctgccgctttgca

SEQ ID NO: 11 - DNA, 21 bp, Artificial
  Synthetic primer
aaaaatggtcgctacagcatc

SEQ ID NO: 12 - DNA, 21 bp, Artificial
  Synthetic primer
aattatgggtctgtaaccacc

SEQ ID NO: 13 - DNA, 21 bp, Artificial
  Synthetic primer
aaatgaatgaaccagattcag

SEQ ID NO: 14 - DNA, 21 bp, Artificial
  Synthetic primer
aaaatcgaacctcagtggcgg

SEQ ID NO: 15 - DNA, 21 bp, Artificial
  Synthetic primer
aatcgaacctcagtggcggcg

SEQ ID NO: 16 - DNA, 21 bp, Artificial
  Synthetic primer
aaaaaatggacaacctgctgc

SEQ ID NO: 17 - DNA, 21 bp, Artificial
  Synthetic primer
aagtgaattcggattgtttgc

SEQ ID NO: 18 - DNA, 21 bp, Artificial
  Synthetic primer
aatctgactgaggggcgggga

SEQ ID NO: 19 - DNA, 21 bp, Artificial
  Synthetic primer
aagccggagctagaggcaggc

SEQ ID NO: 20 - DNA, 21 bp, Artificial
  Synthetic primer
aaaacttgcctactgatcagt

SEQ ID NO: 21 - DNA, 21 bp, Artificial
  Synthetic primer
aacttgcctactgatcagtta

SEQ ID NO: 22 - DNA, 21 bp, Artificial
  Synthetic primer
aagaaactggtgattgttggt

SEQ ID NO: 23 - DNA, 21 bp, Artificial
  Synthetic primer
aaagacatgcttgctcatagt

SEQ ID NO: 24 - DNA, 21 bp, Artificial
  Synthetic primer
aagagaaatcgaaaccgaaaa

SEQ ID NO: 25 - DNA, 21 bp, Artificial
  Synthetic primer
aagaacccaattaagcgcaag

SEQ ID NO: 26 - DNA, 21 bp, Artificial
  Synthetic primer
aaagataaacctctcataatg

SEQ ID NO: 27 - DNA, 21 bp, Artificial
  Synthetic primer
aaacctctcataatgaaggcc

SEQ ID NO: 28 - DNA, 21 bp, Artificial
  Synthetic primer
aaacagcttcagaagcctgac

SEQ ID NO: 29 - DNA, 21 bp, Artificial
  Synthetic primer
aatgctctgaatcctagtctc

SEQ ID NO: 30 - DNA, 21 bp, Artificial
  Synthetic primer
aatgcctggaaattcctcatt

SEQ ID NO: 31 - DNA, 21 bp, Artificial
  Synthetic primer
aaacagaatttcgtgaacagc

SEQ ID NO: 32 - DNA, 21 bp, Artificial
  Synthetic primer
aacatgccgagtttttgcact

SEQ ID NO: 33 - DNA, 21 bp, Artificial
  Synthetic primer
aaacagaacatgccgagtttt

SEQ ID NO: 34 - DNA, 21 bp, Artificial
  Synthetic primer
aaaacttgcctactgatcagt

SEQ ID NO: 35 - DNA, 21 bp, Artificial
  Synthetic primer
aaaggcatagacaacaaaaga

SEQ ID NO: 36 - DNA, 21 bp, Artificial
  Synthetic primer
aacaaaagaaattttattgag

SEQ ID NO: 37 - protein, 79 aa, Homo sapiens
KVQNTCKDNPCGRGQCLITQSPPYYRCVCKHPYTGPSCSQVVPVCRPNPCQNGATCSRHKRRSKFTCACPDQFKGKFCE

SEQ ID NO: 38 - protein, 241 aa, Homo sapiens
YGGFKSTAGKHPWQASLQSSLPLTISMPQGHFCGGALIHPCWVLTAAHCTDIKTRHLKVVLGDQDLKKEEFHEQSFRVEKIFKYSHYNERDEIPHNDIALLKLKPVDGHCALESKYVKTVCLPDGSFPSGSECHISGWGVTETGKGSRQLLDAKVKLIANTLCNSRQLYDHMIDDSMICAGNLQKPGQDTCQGDSGGPLTCEKDGTYYVYGIVSWGLECGKRPGVYTQVTKFLNWIKATIK